(19) United States
(12) Patent Application Publication
(10) Pub. No.: US 2024/0271134 A1
(43) Pub. Date: Aug. 15, 2024

(54) OLIGONUCLEOTIDES AND COMPOSITIONS THEREOF FOR NEUROMUSCULAR DISORDERS

(71) Applicant: MIRECULE, INC., Gaithersburg, MD (US)

(72) Inventors: Anthony SALEH, Gaithersburg, MD (US); Grant BELGARD, Sanford, FL (US); Marton MUNZ, Barcelona (ES); Charles MARUSAK, Gaithersburg, MD (US); Robert PLACE, Gaithersburg, MD (US)

(21) Appl. No.: 18/411,650

(22) Filed: Jan. 12, 2024

Related U.S. Application Data
(63) Continuation of application No. PCT/US2022/073754, filed on Jul. 14, 2022.
(60) Provisional application No. 63/221,568, filed on Jul. 14, 2021.

(51) Int. Cl. C12N 15/113

(52) U.S. Cl. CPC C12N 15/113; C12N 2310/11; C12N 2310/321; C12N 2310/322; C12N 2310/3231; C12N 2310/3513

(57) ABSTRACT

Disclosed herein are engineered DUX4-targeting oligonucleotides for selective inhibition of RNA transcripts associated with a neuromuscular disease such as facioscapulohumeral muscular dystrophy. Also disclosed are vectors containing any of these, pharmaceutical formulations containing any of the these, and kits containing any of the these. Also disclosed herein are methods of selectively inhibiting polypeptide expression and activity by contacting a DUX4-targeting oligonucleotide with an RNA transcript associated with a neuromuscular disease such as facioscapulohumeral muscular dystrophy.

### CROSS-REFERENCE TO RELATED APPLICATIONS

This application is a continuation of International Patent Application No. PCT/US2022/073754, filed Jul. 14, 2022, which claims the benefit of U.S. Provisional Application No. 63/221,568, filed Jul. 14, 2021, the disclosures of which are incorporated herein by reference in their entirety.

### SEQUENCE LISTING

The instant application contains a Sequence Listing which has been submitted electronically in XML format and is hereby incorporated by reference in its entirety. Said XML file, created on Nov. 6, 2023, is named MRC-0004PCT_FINAL_11-6-2023 Corrected.xml and is 37,113,138 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

Certain aspects of this disclosure pertain to nn engineered DUX4-targeting oligonucleotide that is from about 15 to about 25 nucleotides in length, wherein the engineered DUX4-targeting oligonucleotide comprises at least about: 80%, 85%, 90%, or 95% sequence identity to any one of SEQ. ID. NOs: 20,962-42,138. Further, the engineered DUX4-targeting oligonucleotide may be about from about 15 to about 25 nucleotides in length, may comprise at least about 80%, 85%, 90%, or 95% sequence identity to any one of SEQ. ID. NOs: 42,006-42,138.

In certain instances, the engineered DUX4-targeting oligonucleotide of wherein the engineered DUX4-targeting oligonucleotide comprises a DNA nucleotide and an RNA nucleotide. In some cases, this oligonucleotide comprises a DNA nucleotide. In some cases, the oligonucleotide comprises an RNA nucleotide. In certain instances, the oligonucleotide is small interfering RNA (siRNA), a MicroRNA (miRNA), a small nuclear RNA (snRNA), a U spliceosomal RNA (U-RNA), a Small nucleolar RNA (snoRNA), a Piwi-interacting RNA (piRNA), a repeat associated small interfering RNA (rasiRNA), a small rDNA-derived RNA (srRNA), a transfer RNA derived small RNA (tsRNA), a ribosomal RNA derived small RNA (rsRNA), a large non-coding RNA derived small RNA (lncsRNA), or a messenger RNA derived small RNA (msRNA). An oligonucleotide as described above may, in certain cases, comprise at least one locked nucleic acid nucleobase.

The DUX4-targeting oligonucleotide as described above may, bind to the DUX4 coding sequence in an aqueous solution with a predicted melting temperature (Tm) from about 45 to about 65 degrees Celsius wherein the aqueous solution has a pH ranging of from about 7.2 to about 7.6.

Another aspect of this disclosure is a conjugate of a i) DUX4-targeting oligonucleotide as described above wherein the conjugate comprises the oligonucleotide and an antibody, an antibody fragment, a single monomeric variable antibody domain, a naturally occurring ligand, a small molecule, or a peptide; and optionally iii) a linker that links i) to ii).

Another aspect of the disclosure pertains to a vector containing or encoding the conjugate as described herein or an oligonucleotide as described herein. In certain cases, the vector may comprise a viral vector, a nanoparticle vector, a liposomal vector, an exosomal vector, an extracellular vesicle vector, or a combination thereof. The vector may be the liposomal vector. The vector may be the nanoparticle vector. The vector may be the exosomal vector. The vector may be the extracellular vector.

Another aspect of this disclosure pertains to a pharmaceutical composition comprising the engineered DUX4-targeting oligonucleotide of described herein, the conjugate of described herein, a vector as described herein vector of any one of claims 10 to 15, and a pharmaceutically acceptable: excipient, diluent, carrier, or a combination thereof. In certain cases, the pharmaceutically acceptable excipient comprises a buffering agent, a stabilizer, an antioxidant, a diluent, or any combinations thereof. In certain instances, the pharmaceutically acceptable diluent comprises distilled water, deionized water, physiological saline, Ringer's solutions, dextrose solution, a cell growth medium, phosphate buffered saline (PBS), or any combination thereof. The pharmaceutical compositions described herein can be in unit dose form.

Another aspect of this disclosure pertains to a kit comprising the engineered DUX4-targeting oligonucleotide as described herein, the conjugate as described herein, the vector as described herein, or the pharmaceutical composition as described herein and a container. In certain cases, the container may comprise a jar, an ampule, a syringe, a bag, a box, or a combination thereof.

Another aspect of this disclosure is a method of treating a disease or condition in a subject comprising administering to the subject a therapeutically effective amount the pharmaceutical composition as described herein. The disease or condition is a DUX4 mediated disease or condition. The DUX4 mediated disease or condition is facioscapulohumeral muscular dystrophy. The subject may be a subject is in need thereof. The subject may be a human subject in need thereof.

In the method, the administering is in an amount of from about 0.001 mg to about 10,000 mg of the pharmaceutical formulation per kg of body weight of the subject. The administering can be oral, intranasal, rectally, topically, intraocular, intramuscular, intravenous, intraperitoneal, intracardial, subcutaneous, intracranial, intrathecal, or any combination thereof.

The method can use the pharmaceutical composition wherein the pharmaceutical composition a liquid dosage form that is administered at a volume of: about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml, or about 750 ml to 1000 ml. In certain cases, the pharmaceutical composition is in a liquid dosage form, a solid dosage form, an inhalable dosage form, an intranasal dosage form, a liposomal formulation, in the form of a pill, in the form of a capsule, or any combinations thereof.

In certain instances, the administration comprises systemic or local administration. The systemic may be administration, wherein the systemic administration comprises at least one of: a parenteral administration, intravenous administration, subcutaneous administration, intrathecal administration, intraperitoneal administration, intramuscular administration, intravascular administration, infusion, oral administration, inhalation administration, intraduodenal administration, rectal administration, or any combination thereof.

In certain cases, the method further comprises concurrently or consecutively administering a co-therapy.

Another aspect of the disclosure concerns a method of administering the engineered DUX-4 targeting oligonucleotide of described herein, wherein after the administering, the engineered DUX-4 targeting oligonucleotide selectively hybridizes to two different endogenous disease related RNAs wherein one of the two different endogenous disease related RNAs is a DUX4 RNA transcribed from a first genetic loci and one of the two different endogenous disease related RNAs is transcribed from a different genetic loci than the first genetic loci. Still further, in certain cases, the engineered DUX4-targeting oligonucleotide hybridizes to the endogenous disease related RNA that is transcribed from a different genetic loci than the first genetic loci, such that at least 10 continuous oligonucleotides of the engineered DUX4-targeted oligonucleotide hybridize at least two different contiguous sections of contiguous bases that are interrupted by at least one nucleobase. This method can be a method of treating a disease or condition which is a DUX4 mediated disease or condition. The disease or condition can be facioscapulohumeral muscular dystrophy. Upon hybridization between the engineered DUX4-targeting oligonucleotide and the second RNA, the predicted thermal melting point can be about 40 degrees Celsius to about 65 degrees Celsius.

Another aspect of this disclosure is a composition for use in treating a neuromuscular disease comprising an engineered DUX4-targeting oligonucleotide as described herein, a conjugate of as described herein, a vector as described herein, a pharmaceutical composition as described herein and a pharmaceutically acceptable: excipient, diluent, or carrier. The composition can be for use wherein the neuromuscular disease is facioscapulohumeral muscular dystrophy.

### DESCRIPTION OF THE DRAWINGS

FIG. 1 shows genetic modifications that lead to FSHD.

FIG. 2 shows alternately spliced DUX4 transcripts originating from D4Z4 regions.

FIG. 3 shows a schematic of the read coverage from RNA-Seq data of alternately spliced DUX4 transcripts from FSHD and Healthy muscle biopsy tissue.

FIG. 4 shows a schematic of the read coverage from RNA-Seq data of alternately spliced DUX4 transcripts from the Testis.

FIG. 5 shows the serum stability of chemically modified anti-DUX4 ASOs relative to unmodified oligos.

FIGS. 6A-B depict reduction in innate stimulation. FIG. 6A depicts reductions in innate IFNα and TNFα production after exposure of PBMCs to engineered anti-DUX4 ASOs. FIG. 6B depicts reductions in innate immunostimulation for engineered DUX4 ASOs through the Raw-blue cell assay.

FIG. 7 show a DUX4 ASO HTS Assay Design with stable human or mouse myoblasts expressing eGFP with the coding sequence for DUX4 in the 3′ UTR.

FIGS. 8A-B shows knockdown of DUX4 mRNA. FIG. 8A shows therapeutic ASOs have strong knockdown of DUX4 in FSHD myotubes. FIG. 8B shows knockdown of DUX4 and DUX4 induced genes ZSCAN4 and SLC34A2 in FSHD myotubes.

FIG. 9 shows simultaneous knockdown of DUX4 and DBET RNA transcripts in FSHD patient myoblasts by multi-targeted ASOs.

FIG. 10 shows a schematic overview of data analytics to identify FSHD related genes and pathways.

FIG. 11 shows expression of genes representing six FSHD relevant biological functions separated by horizontal gaps (top to bottom): DUX4-regulated, extracellular matrix, cell cycle, immune/inflammatory response, immunoglobulin and muscle development-related

FIGS. 12A-B show exemplary pathways for potential effects by identified co-targets. FIG. 12A shows pathway regulations of Ki-67 in cellular proliferation from Xie et al. (18). FIG. 12B shows induction of IRF5 in inflammatory signaling from Elkon et al. (19).

FIG. 13 shows IRF5 and MKI67 RNA expression in patient biopsy samples.

FIGS. 14A-B shows validation of co-targeted transcripts by multi-targeting ASOs. FIG. 14A shows myoblasts after treatment with ASOs. FIG. 14B shows qRT-PCT results from RNA (DUX4, DBET, IRF5, and MKI67) obtained from the myoblasts treated with ASOs.

FIG. 15 is a diagram showing a method and system as disclosed herein.

FIG. 16 shows a computer control system that is programmed to analyze genetic material.

### DETAILED DESCRIPTION

### Overview

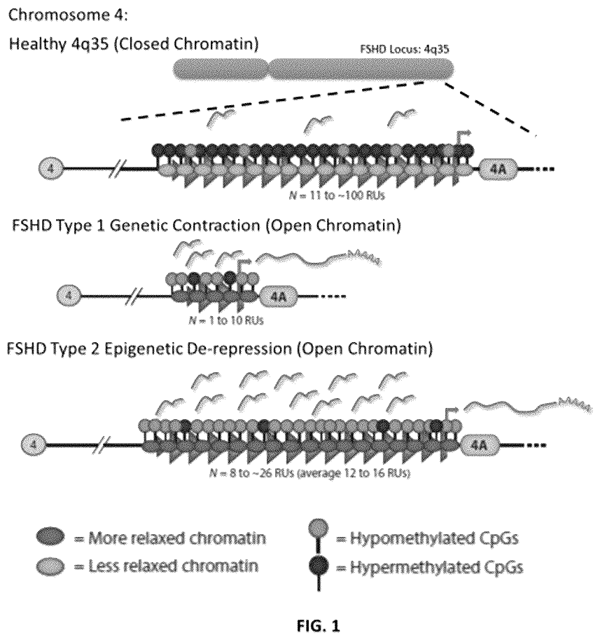

Facioscapulohumeral muscular dystrophy (FSHD) is the third most common form of Muscular Dystrophy (MD) with roughly 40,000 patients presenting with symptoms in the US (1, 2). FSHD Type 1 (FSHD1), which accounts for 95% of all FSHD patients, is the result of a reduction in the number of D4Z4 repeats on chromosome 4q35 from around 100 to less than 11 (3). FSHD Type 2 (SHD2) is the result of a loss of function mutation in the epigenetic factor, Structural Maintenance of Chromosomes flexible Hinge Domain containing 1 (SMCHD1) (3) (FIG. 1). Both inherited mutations result in the hypomethylation of the D4Z4 region which allows for inappropriate expression of double homeobox 4 protein (DUX4) gene encoded within D4Z4. The aberrant expression of DUX4 is severely toxic to muscle tissues, resulting in oxidative stress and apoptosis of muscle cells degrading muscle function (4, 5). FSHD results in progressive weakness in the muscles of the face, shoulders, arms, abdomen, and legs. About 20% of patients are eventually wheelchair-bound (6). When only 1-3 D4Z4 repeats remain, a much more severe and rapidly progressing disorder results (7), with often pediatric-onset (8) and hearing and vision loss (9). Broad scientific consensus exists in the field that if DUX4 expression could be eliminated in muscle tissue, progression of FSHD1 and 2 could be halted (10-12). Several studies have shown that RNA oligonucleotide therapeutics have the potential to directly repress DUX4, reversing muscle pathology in vitro and in mouse models (13-16). However, conservation of the complementary binding site of DUX4 targeted oligonucleotide therapeutics is still a problem.

Oligonucleotide therapeutics (ONT) designed to treat any disorder will be most effective at regulating the targeted transcript if it is perfectly complementary to the target RNA binding site in the disease transcript. In addition, the targeted binding sequence should have low variance between patients with this disorder. Otherwise, patients that have a SNP or mutation in the sequence of the disease gene at the target binding site may not be perfectly complementary with the therapeutic oligonucleotide resulting in less than complete silencing of the disease gene by the ONT. The instant application is the first to solve the problem of determining conserved variant sequences within the DUX4 gene/exons, to identify RNA therapeutics that target clinically significant DUX4 variants, and to generate RNA therapeutics with superior structural modifications for efficacy and stability.

Normally sequence databases including hundreds to thousands of individuals are used to select highly conserved binding sites for oligonucleotide therapeutics (ONTs) (20). However, these databases cannot be used to accurately predict variance in the DUX4 gene. The challenge is to find conserved therapeutic targets of DUX4. Disclosed herein is the solution and generation and validation of DUX4-targeting oligonucleotides. Most public sequence databases utilize DNA fragment sequencing technologies to efficiently and cheaply collect sequence data from populations. This involves fragmentation of long genomic DNA into pieces a few hundred bases in length that are cloned amplified and sequenced. Individual fragments are then mapped to a larger known reference genomic sequence. This technology is known to not be effective at accurately distinguishing or mapping repetitive sequences (21).

The coding regions of the DUX4 gene reside in each D4Z4 repeat on chromosome 4. DNA from a normal individual contains 11-200 copies of D4Z4 on each chromosome 4 (12). In addition. DUX4-containing D4Z4 repeats are found on chromosome 10. However, deletion of D4Z4 repeats on chromosome 10 are not associated with development of facioscapulohumeral muscular dystrophy (FSHD) due to lack of downstream exons 3-5 in the DUX4 coding sequence. Thus, sequence variability found in the chromosome 10 DUX4 coding sequences would not be relevant for design of ONTs. Further. D4Z4 pseudogenes are also found throughout the human genome (22) and significant sequence overlap occurs between DUX4 sequences in D4Z4 and other repetitive DNA sequences encoding DUX family members DUX1-DUX5 (23). This genomic complexity leads to poor mapping of sequenced DNA fragments that overlap with D4Z4 repeats, with little confidence in which genomic loci they originate from. In predicting variation in the DUX4 coding sequence in FSHD patients this creates a problem whereby there is little confidence that the sequence data and that the listed variation can accurately predict conserved sequences in DUX4, as much of the data is contaminated with sequence variation from other genomic locations that do not relate to the disease-causing, shortened D4Z4 repeat array located on one copy of an FSHD patient's chromosome 4. One logical solution would be to use RNA sequence data from muscle biopsies of FSHD patients. As shown in Example 1, this approach does not result in sufficient data to allow for prediction of variability in the DUX4 coding sequence.

This disclosure is the first to solve the problem of determining conserved variant sequences within the DUX4 gene/exons, to identify ONT therapeutics that target clinically significant DUX4 variants, and to generate ONT therapeutics with superior structural modifications for efficacy and stability.

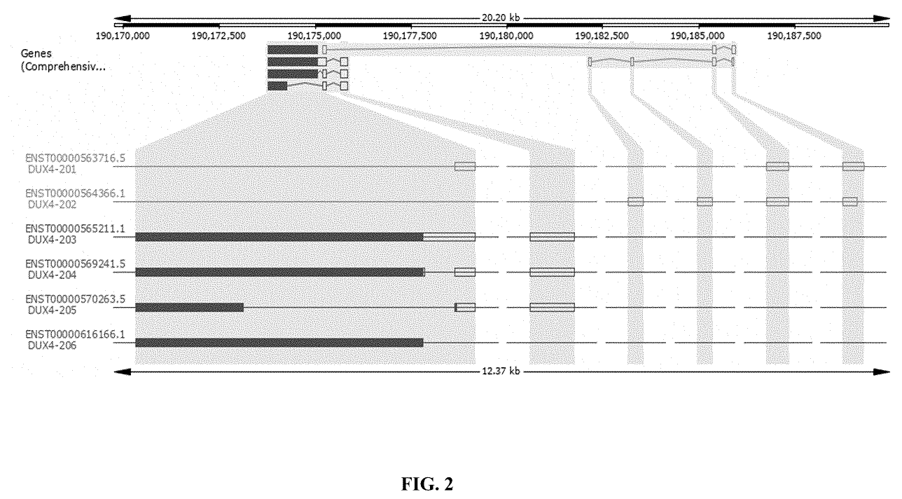

Disclosed herein are sequences representing all regions of the DUX4 coding sequence that are >85% conserved among 206 subjects (Table 4). To identify these regions, the inventors made the surprising discovery, as shown in Example 3, that sufficient read counts could be identified in RNA-seq data by combining RNA-seq data from muscle biopsies for patients into a combined databased with RNA-seq from testis samples. While it is known in the art that low level DUX4 expression is observed in gamete cells in the testis (24), one of skill in the art would not have expected be able to predict DUX4 disease transcript variance from testis RNA sequence as it has been reported that the DUX4 transcript expressed in the testis are differentially spliced and lack regions of exon1, exon2, and exon3 which are included in the muscle specific transcripts for DUX4 that are predicted to cause disease (25) (FIG. 2). By combining the RNA-seq data from these two tissues into a single dataset we were able to generate sufficient read coverage across the entire DUX4 disease gene to predict regions that are greater than 85% conserved and would be able to effectively treat most patients.

Antisense Oligonucleotides (ASOs) that are dependent on RNase H for cleavage and subsequent degradation of complementary RNA, can and do silence many RNAs besides the intended RNA target(26, 27). These non-target RNAs are often referred to as off-target effects. For gapmer ASOs this occurs when the DNA portion of the oligonucleotide causes degradation of unintended RNA off-targets by binding to partially complementary target site and inducing RNAse H cleavage. Careful sequence analysis can identify many of these potential interactions. However, simple sequence alignment does not often accurately predict a real off-target interaction. The inventors have developed a data analysis pipeline to predict and track off-target effects for RNA therapeutics that considers structural motif and binding energy as well to improve predictions (WO2021203043).

The general practice in the field is to avoid off-target effects as much as possible in oligonucleotide design. The novel approach described herein is instead to take a global look at off-targets. The inventors first look for those that would be potentially harmful and cause toxicity by filtering predicted targets through toxicity databases such as Toxnet and Ingenuity Pathway Analysis (IPA). The inventors also consider off-targets that may be related to disease pathways through analysis of transcriptomic profiles of muscle biopsies from FSHD patients, by looking for genes that are significantly overexpressed in subsets, or related to known disease pathways such as inflammation, muscle cell division, or cell death pathways.

This information allows for prioritization of which ASO sequences to synthesize, test and validate. ASOs that demonstrate high knockdown potential and off-targets with high disease relevance are then used to validate knockdown of the off-target transcript in vitro in differentiated myotubes by qRT-PCR.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that can vary depending upon the desired properties sought to be obtained.

As used herein, the term “about” may mean the referenced numeric indication plus or minus: 5%, 10%, 15%, or 20% of that referenced numeric indication. In some instances, “about” may mean the referenced numeric indication plus or minus 15% of that referenced numeric indication. In some instances, “about” may mean the referenced numeric indication plus or minus 20% of that referenced numeric indication. With respect to biological systems or processes, the term can mean within an order of magnitude, within 5-fold, or within 2-fold, of a value. Where particular values are described in the disclosure and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, 99.99% or about 100% of the total amount.

The term “homology” can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed. Any sequence disclosed herein also comprises a sequence with about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the disclosed sequence.

The term “oligonucleotide” can refer to a DNA, RNA, or hybrid nucleic acid sequence, whether chemically modified or not, wherein a single strand, such as for example, in typically the case of DNA, reverse complementarily binds to a target RNA sequence. In the case of RNA, the oligonucleotide may be single stranded such as is typically the case of miRNA, wherein the single strand reverse complementarily binds to a target RNA sequence. In other instances, concerning an RNA oligonucleotide may be double stranded, for example, as is typically the case with siRNA, wherein one strand reverse complimentarily binds to a target RNA sequence.

As used herein, in some instances, the term “targeting” and the term “targeted” can be used interchangeably, for example, an oligonucleotide targeting DUX4 can be a DUX4-targeting oligonucleotide or an oligonucleotide that targets DUX4. It may be a DUX4-targeting oligonucleotide. A targeting sequence can have reverse complementarity to a DUX4 transcript. In some cases, a targeting sequence can have at least partial reverse complementarity to a DUX4 transcript and one or more additional genetic loci, or transcripts thereof. In some cases, the genetic loci

The term “fragment,” as used herein, can be a portion of a sequence, a subset that can be shorter than a full-length sequence. A fragment can be a portion of a gene. A fragment can be a portion of a peptide or protein. A fragment can be a portion of an amino acid sequence. A fragment can be a portion of an oligonucleotide sequence. A fragment can be less than about: 20, 30, 40, 50 amino acids in length. A fragment can be less than about: 2, 5, 10, 20, 30, 40, 50 oligonucleotides in length.

The term “epigenetic marker” as used herein, can be any covalent modification of a nucleic acid base.

The terms “administer,” “administering”, “administration,” and the like, as used herein, can refer to methods that can be used to enable delivery of compounds or compositions to the desired site of biological action. The term “delivery” can include direct application to the affected tissue or region of the body.

The term “subject,” “host.” “individual,” and “patient” are as used interchangeably herein to refer to animals, typically mammalian animals.

The terms “treat.” “treating” or “treatment.” as used herein, may include at least partially: alleviating, abating or ameliorating a disease or condition symptom: preventing an additional symptom: ameliorating or preventing the underlying causes of a symptom; preventing a recurrence of a symptom: inhibiting the disease or condition, e.g., at least partially arresting a development of the disease or condition: relieving a disease or condition: causing regression of a disease or condition: relieving a condition caused by the disease or condition: or stopping a symptom of the disease or condition either prophylactically, therapeutically or both.

As used herein, “agent” or “biologically active agent” may refer to a biological, pharmaceutical, or chemical compound or a salt of any of these structures.

The term “tissue” as used herein, can be any tissue sample. A tissue can be a tissue suspected or confirmed of having a disease or condition.

The term “mammalian cell” can refer to any mammalian cell, typically a human cell.

### Engineered DUX4-Targeting Oligonucleotides

The disclosure herein provides for the therapeutic targeting of RNA transcripts comprising a select DUX4 target location. Two major methods are employed in RNA medicine: double stranded RNA-mediated interference (RNAi) and antisense oligonucleotides (ASO). Broadly speaking. RNAi may operate by activating ribonucleases which, along with other enzymes and complexes, coordinately degrade the RNA after the original RNA target has been cut into smaller pieces. Antisense oligonucleotides may bind to their target nucleic acid via Watson-Crick base pairing, and inhibit or alter gene expression via steric hindrance, splicing alterations, initiation of target degradation, or other events.

In certain aspects of the disclosure, oligonucleotide therapeutics (ONT) may be designed to treat any disorder amenable to regulating a targeted transcript. In certain aspects, the treatment is with one or more substantially or perfectly complementary ASOs with regard to a target RNA binding site of a disease having a transcript in need of downregulation. In certain cases, the oligonucleotide therapeutics are primarily DNA, in other cases, the oligonucleotides are primarily RNA. Generally, ASOs that efficiently target DUX4 can bind to the fusion transcript and induce degradation through RNAse H.

In other aspects of the disclosure, interfering RNA such as siRNA or miRNA comprising a sequence which is complementary to a DUX4 RNA transcript may be designed to treat any disorder amenable to regulating such a targeted transcript. In certain aspects, a siRNA is double stranded with one strand being complementary. RISC uses the guide strand of miRNA or siRNA to target complementary 3′-untranslated regions (3′UTR) of mRNA transcripts via Watson-Crick base pairing, allowing it to regulate gene expression of the mRNA transcript in a number of ways such as mRNA degradation, thereby preventing or reducing protein expression of the selected mRNA.

Oligonucleotides as mentioned, may comprise miRNA. Such miRNA may contain one or more sequence modifications, one or more chemical modifications, or a combination thereof that can: enhance stability of the miRNA: substantially reduce or eliminate immune stimulation (such as via the innate immune response): improve pharmacological activity of the miRNA: retain poly-targeting effects of the miRNA: or any combination thereof.

Nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified or naturally occurring bases. Likewise, an RNA transcript with the sequence “AUCGAUCG” encompasses any corresponding DNA sequence such as “ATCGATCG”. Nucleic acid sequences herein also comprise sequences comprising at least about: 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the disclosed sequence.

In certain cases, an oligonucleotide construct may comprise a first strand comprising the DUX4-targeting oligonucleotide and a second strand comprising a sequence complementary to at least a portion of the DUX4-targeting oligonucleotide. The second strand may be complementary to at least about 10%, 20%, 30%, 40%, 50%, 60%, 70%, 80%, 90% or more of the first strand. The second strand may be complementary to at least about: 5, 10, 15, or 20 contiguous bases of the first strand. An oligonucleotide may comprise an end overhang, such as a 5′ end or a 3′ end. The first strand, the second strand or a combination thereof may comprise one or more chemical modifications. At least about 10%, 20%, 30%, 40%, 50%, 60%, 70%, 80%, 90% of bases of a first strand, a second strand, or a combination thereof may comprise a chemical modification. The first strand, the second strand or a combination thereof may comprise one or more sugar modifications. At least about 10%, 20%, 30%, 40%, 50%, 60%, 70%, 80%, 90% of bases of a first strand, a second strand, or a combination thereof may comprise a sugar modification. A sugar modification may comprise a glycosylated base. In some cases, a base of a nucleotide may be glycosylated with a glycan. The first strand, the second strand or a combination thereof may comprise a combination of bases having a chemical modification and a sugar modification.

In some cases, an oligonucleotide as described herein such as a DUX4-targeting oligonucleotide or salt thereof may be from about 5 to about 50 nucleotides in length. In some cases, the DUX4-targeting oligonucleotide or salt thereof may be from about 5 to about 40) nucleotides in length. In some cases, the DUX4-targeting oligonucleotide or salt thereof may be from about 5 to about 30 nucleotides in length. In some cases, the DUX4-targeting oligonucleotide or salt thereof may be from about 5 to about 25 nucleotides in length. In some cases, the DUX4-targeting oligonucleotide or salt thereof may be from about 5 to about 60) nucleotides in length. In some cases, the DUX4-targeting oligonucleotide or salt thereof may be from about 5 to about 80 nucleotides in length. In some cases, the DUX4-targeting oligonucleotide or salt thereof may be from about 5 to about 100 nucleotides in length. In some cases, the DUX4-targeting oligonucleotide or salt thereof may be from about 5 to about 200 nucleotides in length.

In certain other cases, an interfering RNA may be a regulatory non-coding RNA (ncRNA) comprising short non-coding RNA sequences expressed in a genome that regulates expression or function of other biomolecules in mammalian cells. An ncRNA is generally <200 nucleotides in length and may be single stranded or double stranded and may form non-linear secondary or tertiary structures. An ncRNA may comprise exogenously derived small interfering RNA (siRNA), MicroRNA (miRNA), small nuclear RNA (snRNA). U spliceosomal RNA (U-RNA). Small nucleolar RNA (snoRNA). Piwi-interacting RNA (piRNA), repeat associated small interfering RNA (rasiRNA), small rDNA-derived RNA (srRNA), transfer RNA derived small RNA (tsRNA), ribosomal RNA derived small RNA (rsRNA), large non-coding RNA derived small RNA (lncsRNA), or a messenger RNA derived small RNA (msRNA).

A DUX4-targeting oligonucleotide may comprise DNA. RNA or a mixture thereof. In some cases, a DUX4-targeting oligonucleotide may comprise a plurality of nucleotides. In some cases, a DUX4-targeting oligonucleotide may comprise an artificial nucleic acid analogue. In some cases, a DUX4-targeting oligonucleotide may comprise DNA, may comprise cell-free DNA, cDNA, fetal DNA, viral DNA, or maternal DNA. In some cases, a DUX4-targeting oligonucleotide can comprise an shRNA, or siRNA, an ncRNA mimic, a short-harpin RNA (shRNA), a dicer-dependent siRNA (di-siRNA), an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), or an exon skipping oligonucleotide. In some cases, a DUX4-targeting oligonucleotide may comprise a completely synthetic miRNA. A completely synthetic miRNA is one that is not derived or based upon an ncRNA. Instead, a completely synthetic miRNA may be based upon an analysis of multiple potential target sequences or may be based upon isolated natural non-coding sequences that are not ncRNAs.

### Modified Oligonucleotides

In some cases, a second strand may comprise a chemically modified base of a nucleotide. In some cases, a subset of bases of the second strand may be chemically modified, such as from about 1% to about 5% of bases, from about 1% to about 10% of bases, from about 1% to about 20% of bases, from about 1% to about 30% of bases, from about 1% to about 40% of bases, from about 1% to about 50% of bases, from about 1% to about 60% of bases, from about 1% to about 70% of bases, from about 1% to about 80% of bases, or from about 1% to about 90% of bases, or more. A second strand as described herein may be chemically modified in the same manner as described herein for the DUX4-targeting oligonucleotide.

An oligonucleotide may comprise a sugar modification. An oligonucleotide may comprise a plurality of sugar modifications. A sugar modification may comprise a glucose or derivative thereof. A sugar modification may comprise a ribose or deoxyribose. A sugar modification may comprise a monosaccharide, a disaccharide, a trisaccharide or any combination thereof.

In some cases, a ribonucleotide or a deoxynucleotide, may be modified, such as the base component, the sugar (ribose) component, the phosphate component forming the backbone of the DUX4-targeting oligonucleotide, or any combination thereof, by a chemical modification as described herein.

An oligonucleotide such as a DUX4-targeting oligonucleotide may comprise a chemical modification. An oligonucleotide may comprise a plurality of chemical modifications. An oligonucleotide may comprise a plurality of chemical modifications within a portion of an oligonucleotide, such as a terminal end. A chemical modification may comprise a methyl group, a fluoro group, a methoxyethyl group, an ethyl group, an amide group, an ester group, more than one of any of these, or any combination thereof. A chemical modification may comprise a chemically modified nucleotide such as guanosine, uridine, adenosine, thymidine or cytosine including, any natively occurring or non-natively occurring guanosine, uridine, adenosine, thymidine or cytidine that has been altered chemically, for example by acetylation, methylation, hydroxylation, etc., including 1-methyl-adenosine, 1-methyl-guanosine, 1-methyl-inosine, 2,2-dimethyl-guanosine, 2,6-diaminopurine, 2′-amino-2′-deoxyadenosine, 2 ‘-amino-2’-deoxycytidine, 2′-amino-2′-deoxyguanosine, 2 ‘-amino-2’-deoxyuridine, 2-amino-6-chloropurineriboside, 2-aminopurineriboside, 2′-araadenosine, 2′-aracytidine, 2′-arauridine, 2′-azido-2′-deoxyadenosine, 2′-azido-2′-deoxycytidine, 2′-azido-2 ‘-deoxyguanosine, 2’-azido-2′-deoxyuridine, 2-chloroadenosine, 2′-fluoro-2′-deoxyadenosine, 2 ‘-fluoro-2’-deoxy cytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2-O-methyl-2′-deoxyuridine, 2′-O-methyl-5-methyluridine, 2′-O-methylinosine, 2′-O-methylpseudouridine, 2-thiocytidine, 2-thiocytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thiouracil, 5-carboxymethylamonomethyl-uracil, 5-chloro-ara-cytosine, 5-fluorouridine, 5-iodouridine, 5-methoxycarbonylmethyl-uridine, 5-methoxy-uridine, 5-methyl-2-thiouridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl] carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, xylo-adenosine, or any combination thereof. The preparation of such variants is known to the person skilled in the art, for example from U.S. Pat. No. 4,373,071, 4,401,796, 4,415,732, 4,458,066, 4,500,707, 4,668,777, 4,973,679, 5,047,524, 5,132,418, 5,153,319, 5,262,530 or 5,700,642.

In some cases an oligonucleotide such as a DUX4-targeting oligonucleotide may comprise a chemically modified nucleotide such as 2-amino-6-chloropurineriboside-5′-triphosphate, 2-aminopurine-riboside-5′-triphosphate, 2-aminoadenosine-5′-triphosphate, 2 ‘-amino-2’-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, puromycin-5′-triphosphate, xanthosine-5′-triphosphate, or any combination thereof.

In some cases, an oligonucleotide such as a DUX4-targeting oligonucleotide may comprise a chemically modified nucleotide such as pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, or any combination thereof.

In some cases, an oligonucleotide such as a DUX4-targeting oligonucleotide, DUX4-targeting oligonucleotide may comprise a chemically modified nucleotide such as 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, or any combination thereof.

In some cases, an oligonucleotide such as a DUX4-targeting oligonucleotide may comprise a chemically modified nucleotide such as 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, or any combination thereof.

In some cases, an oligonucleotide such as a DUX4-targeting oligonucleotide may comprise a chemically modified nucleotide such as inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, or any combination thereof.

In some cases, an oligonucleotide such as a DUX4-targeting oligonucleotide may comprise a chemically modified nucleotide such as 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine, or any combination thereof.

In some cases, an oligonucleotide such as a DUX4-targeting oligonucleotide may comprise a chemically modified nucleotide, which may be chemically modified at the 2′ position. The chemically modified oligonucleotide may comprise a substituent at the 2′ carbon atom, wherein the substituent may comprise a halogen, an alkoxy group, a hydrogen, an aryloxy group, an amino group or an aminoalkoxy group, such as a 2′-hydrogen (2′-deoxy), 2′-O-methyl, 2′-O-methoxyethyl, 2′-fluoro, 2′ Methoxyethyl, 2′-fluoro, a locked nucleic acid (LNA), or any combination thereof.

Another chemical modification to an oligonucleotide such as a DUX4-targeting oligonucleotide (such as one involving the 2′ position of a nucleotide) may be a locked nucleic acid (LNA) nucleotide, an ethylene bridged nucleic acid (ENA) nucleotide, an (S)-constrained ethyl (cEt) nucleotide, a bridged nucleic acid (BNA) or any combination thereof. A backbone modification may lock the sugar of the modified nucleotide into a preferred northern conformation. In some case, a presence of this type of modification in the target sequence of the DUX4-targeting oligonucleotide may allow for stronger and faster binding of the DUX4-targeting oligonucleotide sequence to the target site.

In some cases, an oligonucleotide such as DUX4-targeting oligonucleotide may comprise at least one chemically modified nucleotide, wherein the phosphate backbone, which may be incorporated into the DUX4-targeting oligonucleotide, may be modified. One or more phosphate groups of the backbone may be modified, for example, by replacing one or more of the oxygen atoms with a different substituent. Further, the modified nucleotide may include a full replacement of an unmodified phosphate moiety with a modified phosphate as described herein. Examples of modified phosphate groups may include a phosphorothioate, a methylphosphonate, a phosphoroselenate, a borano phosphate, a borano phosphate ester, a hydrogen phosphonate, a phosphoroamidate, an alkyl phosphonate, an aryl phosphonate or a phosphotriester. The phosphate linker may also be modified by the replacement of a linking oxygen with nitrogen (bridged phosphoroamidates), sulfur (bridged phosphorothioates) and carbon (bridged methylene-phosphonates).

In some cases, an oligonucleotide such as a DUX4-targeting oligonucleotide may comprise a sugar modification. The sugar modification may comprise a conjugate, such as a linker. In some cases, the DUX4-targeting oligonucleotide may comprise one or more linker groups. The DUX4-targeting oligonucleotide may be linked to an antibody, a protein, a lipid, an aptamer, a small molecule, a drug, or any combination thereof. A linker may form a covalent bond. The DUX4-targeting oligonucleotide may be linked to one or more oligonucleotides, such as a second DUX4-targeting oligonucleotide via a linker. In some cases, the linker may be a cleavable linker. In some cases, a linker may comprise an azide linker. The DUX4-targeting oligonucleotide may comprise a base of a nucleotide that is glycosylated with a glycan. In some cases, the DUX4-targeting oligonucleotide may comprise an abasic site, such as a nucleotide lacking an organic base. In some cases, the abasic nucleotide may comprise a chemical modification as described herein, such as at the 2′ position of the ribose. In some cases, the 2′ C atom of the ribose may be substituted with a substituent such as a halogen, an alkoxy group, a hydrogen, an aryloxy group, an amino group or an aminoalkoxy group, in some cases from 2′-hydrogen (2′-deoxy), 2′-O-methyl, 2′-O-methoxyethyl or 2′-fluoro. In some cases, an abasic site nucleotide may comprise structures 1A or 1B:

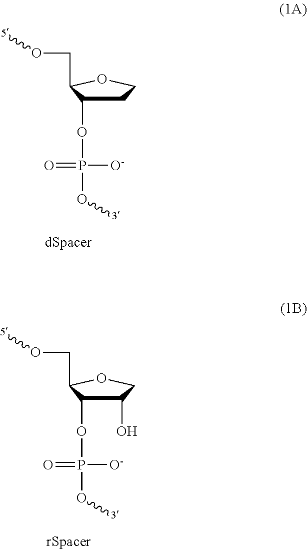

In some cases, an oligonucleotide such as a DUX4-targeting oligonucleotide may be modified by the addition of a “5′-CAP” structure. A 5′-cap may be an entity, such as a modified nucleotide entity, which may ‘cap’ the 5′-end of a mature miRNA. A 5′-cap may typically be formed by a modified nucleotide, particularly by a derivative of a guanine nucleotide. In some cases, the 5′-cap may be linked to the 5′-terminus of the DUX4-targeting oligonucleotide via a 5′-5′-triphosphate linkage. A 5′-cap may be methylated, e.g. m7GpppN, wherein N may be the terminal 5′ nucleotide of the nucleic acid carrying the 5′-cap, such as the 5′-end of an RNA. A 5′-cap structure may include glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′, 4′-seco nucleotide, acyclic 3,4-dihydroxybutyl nucleotide, acyclic 3,5 di hydroxy pentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. In some cases, a modified 5′-CAP structure may comprise a CAP1 (methylation of the ribose of the adjacent nucleotide of m7G), CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, or 2-azido-guanosine.

In some cases, an oligonucleotide such as a DUX4-targeting oligonucleotide, may include a covalent modification may comprise adding a methyl group, a hydroxymethyl group, a carbon atom, an oxygen atom, or any combination thereof to one or more bases of a nucleic acid sequence. In some cases, a covalent modification may comprise changing an oxidation state of a molecule associated with a nucleic acid sequence, such as an oxygen atom, or a combination thereof. A covalent modification may occur at any base, such as a cytosine, a thymine, a uracil, an adenine, a guanine, or any combination thereof. In some cases, an epigenetic modification may comprise an oxidation or a reduction. A nucleic acid sequence may comprise one or more epigenetically modified bases. An epigenetically modified base may comprise any base, such as a cytosine, a uracil, a thymine, adenine, or a guanine. An epigenetically modified base may comprise a methylated base, a hydroxymethylated base, a formylated base, or a carboxylic acid containing base or a salt thereof. An epigenetically modified base may comprise a 5-methylated base, such as a 5-methylated cytosine (5-mC). An epigenetically modified base may comprise a 5-hydroxymethylated base, such as a 5-hydroxymethylated cytosine (5-hmC). An epigenetically modified base may comprise a 5-formylated base, such as a 5-formylated cytosine (5-fC). An epigenetically modified base may comprise a 5-carboxylated base or a salt thereof, such as a 5-carboxylated cytosine (5-caC). In some cases, an epigenetically modified base may comprise a methyltransferase-directed transfer of an activated group (mTAG).

An epigenetically modified base may comprise one or more bases or a purine (such as Structure 1) or one or more bases of a pyrimidine (such as Structure 2). An epigenetic modification may occur at one or more of any positions. For example, an epigenetic modification may occur at one or more positions of a purine, including positions 1, 2, 3, 4, 5, 6, 7, 8, 9, as shown in Structure 1. In some cases, an epigenetic modification may occur at one or more positions of a pyrimidine, including positions 1, 2, 3, 4, 5, 6, as shown in Structure 2.

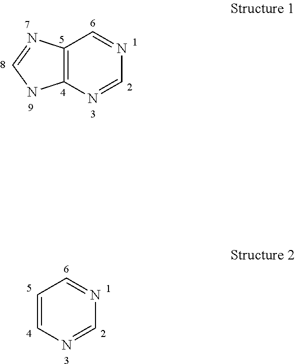 [SMILES: c1ncc2nc[nH]c2n1 ; c1cncnc1]

A nucleic acid sequence may comprise an epigenetically modified base. A nucleic acid sequence may comprise a plurality of epigenetically modified bases. A nucleic acid sequence may comprise an epigenetically modified base positioned within a CG site, a CpG island, or a combination thereof. A nucleic acid sequence may comprise different epigenetically modified bases, such as a methylated base, a hydroxymethylated base, a formylated base, a carboxylic acid containing base or a salt thereof, a plurality of any of these, or any combination thereof.

In some cases, a DUX4-targeting oligonucleotide or salt thereof, when chemically modified, may be of formula: Guide Pattern 1, Guide Pattern 2, or Guide Pattern 3 as shown in Table 1.

TABLE 1
Chemical Modification Formula

| Pattern | Sequence(5′-3′) |
|---|---|
| 1 | {N}*{N}*(n*)ₐ{N}*{N} |
| 2 | {N}*{N}*(n*)_{b}{N}*n*{N}*{N} |
| 3 | {N}*{N}*{N}*(n*)_{b}{N}*{N}*{N} |
| 4 | {N}*{N}*n*{N}*(n*)_{c}{N}*{N}*n*n*{N}*{N} |
| 5 | <N>*<N>*<N>*<N>*(n*)_{d}<N>*<N>*<N>*<N> |
| 6 | CAP-{N}*{N}*(n*)ₐ{N}*{N} |
| 7 | CAP-{N}{N}(n*)ₑn{N}{N} |
| 8 | CAP-{N}mp{N}(n*)ₑn{N}mp{N} |
| 9 | CAP-{N}{N}{N}*(n*)_{b}{N}*{N}{N} |
| 10 | CAP-<N>*<N>*<N>*<N>*(n*)_{d}<N>*<N>*<N>*<N> |

As shown in Table 4, N and n may be any natural or non-natural nucleotide; {N} may be an LNA; [N] may be a BNA; <N> may be a 2′-methyloxyethyl-modified uracil, guanine, adenine, or cytosine; * may be a phosphothionate-modified backbone; mp may be a methylphosphonate-modified backbone; CAP may be 5′-terminal methyl group (5′-OMethyl) or alkylamino group such as amino-carbon 6 chain (5′-Amino C6); a may be from 10-26; b may be from 8-24; c may be from 4-20; d may be from 5-22; e may be from 9-25.

In some cases, an oligonucleotide such as DUX4-targeting oligonucleotide may comprise a chemical modification, to a base or a sugar of the DUX4-targeting oligonucleotide, relative to a natural base or sugar. In some cases, the DUX4-targeting oligonucleotide may comprise more than one chemical modification, such as a plurality of chemical modifications. A portion of bases or a portion of sugars of the DUX4-targeting oligonucleotide may comprise one or more chemical modifications. In some cases, about: 10%, 20%, 30%, 40%, 50%, 60%, 70%, 80%, 90% or more of bases or sugars in a DUX4-targeting oligonucleotide may be chemically modified.

In some cases, a DUX4-targeting oligonucleotide may be engineered or modified to increase a specificity for an RNA sequence among a plurality of RNA sequences. A DUX4-targeting oligonucleotide may be modified to significantly increase a specificity for an RNA sequence among a plurality of RNA sequences. Increased specificity may be compared to a comparable oligonucleotide that may not be engineered or may be compared to a comparable oligonucleotide that may be engineered or modified in a different way. A specificity may be increased by at least about: 2%, 5%, 10%, 15%, 20%, 25%, 30%, 35%, 40%, 45%, 50%, 55%, 60%, 65%, 70%, 75%, 80%, 85%, 90%, 95% or more as compared to a comparable oligonucleotide. A DUX4-targeting oligonucleotide may be engineered or modified to increase a specificity for a first RNA sequence as compared to a second RNA sequence.

### Research and Discovery of DUX4-Targeting Oligonucleotides

To identify target DUX4 variants the identity between a reference sequence (query sequence, i.e., a sequence as described herein) and a subject sequence, also referred to as a global sequence alignment, may be determined using the FASTDB computer program based on the algorithm of Brutlag et al. (Comp. App. Biosci. 6:237-245 (1990)). In some cases, parameters for a particular aspect in which identity is narrowly construed, used in a FASTDB amino acid alignment, may include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. If the subject sequence is shorter than the query sequence due to N- or C-terminal deletions, not because of internal deletions, a manual correction may be made to the results to take into consideration that the FASTDB program does not account for N- and C-terminal truncations of the subject sequence when calculating global percent identity. For subject sequences truncated at the N- and C-termini, relative to the query sequence, the percent identity may be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned may be determined by results of the FASTDB sequence alignment. This percentage may be then subtracted from the percent identity, calculated by the FASTDB program using the specified parameters, to arrive at a final percent identity score. This final percent identity score may be used for the purposes of this aspect. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90-residue subject sequence may be aligned with a 100-residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90-residue subject sequence is compared with a 100-residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

In order to evaluate all different OTN positions, windows of sizes 15 bp, 16 bp, 17 bp, 18 bp, 19, and 20 bp were generated with 1 bp sliding in the reference sequence across the DUX4 gene chr4:190,173,774-190,185,942. For each window the reverse complement (antisense) sequence of the reference was also reported so it could be used directly for OTN design. RNA-Seq BAM files of all the samples were merged into a single BAM file using the Pysamstats v1.1.2 tool https://github.com/alimanfoo/pysamstats and custom Python scripts were used to obtain the reference base frequencies and read depth at each genomic position in the merged BAM files. Mean coverage was defined as the average number of reads covering each base of the window. A minimum conservation score was calculated for each OTN window representing the base with the lowest conservation. Average melting temperature (Tm) was calculated for the resulting OTN/target RNA duplex with the Primer3 v2.4.0 R tool (39), with default parameters, using the nearest neighbor model. Two melting temperature (TM) values were reported based on the different salt correction formula defined by SantaLucia 1998 (40) and Owczarzy et al. 2004 (41). We then filtered this data for OTN and OTN binding sites in DUX4 15-20 bp in length with mean coverage of >50, a minimum conservation >85% among individuals in the study, and an Average TM of 45-65° C. All resulting OTN sequences and paired DUX4 target site sequences, all represented in DNA form, are submitted as an sequence listing file encompassing SEQ. ID. NOs 1-2X,XXX. We included them in the disclosure as they represent a valuable resources for any effort to develop OTNs to treat DUX4 mediated disorders. These DUX4-targeting oligonucleotide or salt thereof, when chemically modified or when not chemically modified, may have at least 90% sequence identity to any one of SEQ. ID. NOs: 41,923-42,115. In certain instances, a DUX4-targeting oligonucleotide or salt thereof may comprise at least about 80% sequence identity to an oligonucleotide of any one of SEQ. ID. NOs: 41,923-42,115. For example, a DUX4-targeting oligonucleotide or salt thereof may comprise at least about 90% sequence identity to an oligonucleotide of any one of SEQ. ID. NOs: 41,923-42,115. In some cases, a DUX4-targeting oligonucleotide or salt thereof may comprise from about 80% to 100% sequence identity to an oligonucleotide of any one of SEQ. ID. NOs: 41,923-42,115. In some cases, a DUX4-targeting oligonucleotide or salt thereof may comprise from about 85% to 100% sequence identity to an oligonucleotide of any one of SEQ. ID. NOs: 41,923-42,115. In some cases, a DUX4-targeting oligonucleotide or salt thereof may comprise at least 80% sequence identity to at least about 10 contiguous bases of any one of SEQ. ID. NOs: 41,923-42,115. In some cases, a DUX4-targeting oligonucleotide or salt thereof may comprise at least 85% sequence identity to at least about 10 contiguous bases of any one of SEQ. ID. NOs: 41,923-42,115. In some cases, the DUX4-targeting oligonucleotide may comprise at least about: 70%, 75%, 80%, 85%, 90%, 95%, 96%, 97%, 98%, or 99% sequence identity to any one of SEQ. ID. NOs: 41,923-42,115, or any combinations thereof.

Additionally, analysis provided the ability to produce numerous DUX4-targeting oligonucleotides, as shown in Table. 2 without chemical modifications (SEQ. ID. NOs: 41,923-41,982) and with chemical modifications (SEQ. ID. Nos: 41,983-42,115) which are all represented in DNA form. Regarding the chemical modified DUX4-targeting oligonucleotides, as shown in Table 2, {N} may be an LNA; [N] may be a BNA; (N) may be a 2′-methyloxyethyl-modified uracil, guanine, adenine, or cytosine; * may be a phosphothionate-modified backbone; mp may be a methylphosphonate-modified backbone; Amino C6− may be 5′ amino-carbon 6 chain. Additionally, certain DUX4-targeting oligonucleotides as shown in Table 2, were able to interact with multiple subsequences of the target DUX4 mRNA as shown in Table 3 also submitted in xml file. In addition, any of the chemically modified oligonucleotides could be synthesized with a 5′ amino-carbon 6 chain even if not displayed in the table with retention of activity. Additional targeted RNAs are only listed next to the unmodified sequence of the oligonucleotide, they are not repeated for chemically modified versions of the same sequence although they would still be targeted by that sequence.

TABLE 2
DUX4-targeting oligonucleotides which hybridizes with additional disease
related RNAs

| | | SEQ | |
|---|---|---|---|
| | | ID | Additional Targeted |
| Oligo | Sequence (5′-3′) | NO: | RNA SEQ ID Nos: |
| AS- | GCCATCGCGGGTAGCC | 41923 | |
| DX- | | | |
| 001 | | | |
| AS- | TGTCGGGAGGGCCATC | 23534 | 42274; 42482; 42169; |
| DX- | | | 42566 |
| 002 | | | |
| AS- | TCCAAACGAGTCTCCG | 41924 | 42885 |
| DX- | | | |
| 003 | | | |
| AS- | GATTCTGAAACCAGAT | 41925 | 42331; 42600 |
| DX- | | | |
| 004 | | | |
| AS- | GCGGGCGCCCTGCCAC | 41926 | 42178; 42790; 42711; |
| DX- | | | 42539; 42406; 42161; |
| 005 | | | 42507; 42817; 42196; |
| | | | 42390; 42563; 42167; |
| | | | 42504; 42426; 42464 |
| AS- | TCATCCAGCAGCAGGC | 41927 | 42694; 42734; 42493; |
| DX- | | | 42284; 42481; 42636; |
| 006 | | | 42352; 42705; 42147; |
| | | | 42235; 42775; 42219; |
| | | | 42752; 42633; 42758; |
| | | | 42166; 42559; 42733; |
| | | | 42651; 42413; 42581; |
| | | | 42479; 42827; 42595; |
| | | | 42365; 42260; 42230; |
| | | | 42866; 42144; 42725; |
| | | | 42143; 42678; 42362; |
| | | | 42172; 42206; 42173; |
| | | | 42884; 42832; 42141; |
| | | | 42480; 42590; 42151; |
| | | | 42168; 42236; 42321; |
| | | | 42248; 42586; 42767; |
| | | | 42485 |
| AS | TAGCCAGCCAGGTGTT | 23789 | 42287; 42359; 42577; |
| DX- | | | 42157; 42728; 42856; |
| 007 | | | 42848; 42443; 42524; |
| | | | 42202 |
| AS- | CAGCGTCGGAAGGTGG | 23942 | 42154; 42244; 42676 |
| DX- | | | |
| 008 | | | |
| AS- | TAGACAGCGTCGGAAG | 23946 | |
| DX- | | | |
| 009 | | | |
| AS- | ATAGGATCCACAGGGA | 23981 | 42214; 42806; 42215; |
| DX- | | | 42432; 42813; 42412; |
| 010 | | | 42392; 42502 |
| AS- | TCTATAGGATCCACAG | 41928 | 42689 |
| DX- | | | |
| 011 | | | |
| AS- | GCACTAATCATCCAGG | 25364 | 42841; 42774; 42176; |
| DX- | | | 42746 |
| 012 | | | |
| AS- | CAGCGTCGGAAGGTG | 21509 | 42429; 42210; 42619; |
| DX- | | | 42617; 42153; 42242; |
| 014 | | | 42675 |
| AS- | CCTAGACAGCGTCGGAAGGT | 38051 | |
| DX- | | | |
| 015 | | | |
| AS- | ATAGGATCCACAGGGAGG | 30389 | 42760; 42492 |
| DX- | | | |
| 018 | | | |
| AS- | CGGCTCTGGGATCCCCGG | 41929 | 42310; 42826; 42744 |
| DX- | | | |
| 019 | | | |
| AS- | GCAGTTCTCCGCGGAG | 41930 | 42290; 42707; 42250; |
| DX- | | | 42343; 42171; 42148 |
| 020 | | | |
| AS- | GGGGCGGAGACACGCCC | 41931 | 42295; 42648; 42715; |
| DX- | | | 42717 |
| 021 | | | |
| AS- | AGAAGGCAGGAATCCCAG | 30245 | 42688; 42687; 42685; |
| DX- | | | 42686; 42307; 42592; |
| 022 | | | 42275; 42584; 42663; |
| | | | 42188 |
| AS- | GCAGGAATCCCAGGCCGG | 41932 | 42386; 42306; 42421 |
| DX- | | | |
| 023 | | | |
| AS- | CTCCGCGGAGTGGAGT | 41933 | 42289; 42149; 42878; |
| DX- | | | 42783; 42661; 42423 |
| 024 | | | |
| AS- | GGAGTCTCTCACCGGGCC | 41934 | 42308 |
| DX- | | | |
| 025 | | | |
| AS- | AGAGGCCAGCGAGCTCCC | 41935 | 42221; 42329; 42773; |
| DX- | | | 42197; 42621 |
| 026 | | | |
| AS- | GGCTCTGGGATCCCCGGG | 41936 | 42309; 42454; 42824; |
| DX- | | | 42743; 42763 |
| 027 | | | |
| AS- | CAGAGAGGCCAGCGAGCT | 30302 | 42811; 42672; 42330; |
| DX- | | | |
| 028 | | | 42340; 42175; 42371 |
| AS- | GACAGCGTCGGAAGGTGG | 30341 | |
| DX- | | | |
| 029 | | | |
| AS- | GTAACTCTAATCCAGGTT | 30365 | |
| DX- | | | |
| 030 | | | |
| AS- | GACATTCAGCCAGAAT | 23921 | 42401; 42703; 42770; |
| DX- | | | 42568; 42508; 42328; |
| 031 | | | 42450 |
| AS- | ACAAGGGCACAGAGAGGC | 30310 | 42868; 42254; 42194; |
| DX- | | | 42554 |
| 032 | | | |
| AS- | GAGCTCCCTTGCACGTCA | 41937 | |
| DX- | | | |
| 033 | | | |
| AS- | CCGTCCAACCCCGCGTC | 41938 | |
| DX- | | | |
| 034 | | | |
| AS- | CCTAAAGCTCCTCCAGCA | 30205 | 42253; 42302; 42536 |
| DX- | | | |
| 035 | | | |
| AS- | GCGAGGCGGCCTCTTCCG | 41939 | 42297 |
| DX- | | | |
| 036 | | | |
| AS- | GCCTCCAGCTCCCCCGGG | 41940 | 42296; 42793; 42472; |
| DX- | | | 42785; 42348; 42189; |
| 037 | | | 42673; 42227; 42203; |
| | | | 42851; 42198 |
| AS- | GGTGTCGGGAGGGCCAT | 41941 | 42483; 42589; 42394 |
| DX- | | | |
| 038 | | | |
| AS- | GCCTCAGCTGGCGTGA | 23626 | 42797; 42666; 42427; |
| DX- | | | 42357; 42660; 42653; |
| 039 | | | 42858; 42531 |
| AS- | CTGGGCCAGCCGTTCTCT | 41942 | 42370 |
| DX- | | | |
| 040 | | | |
| AS- | GGGCCAGCCGTTCTCTGG | 41943 | 42269 |
| DX- | | | |
| 041 | | | |
| AS- | GCTCCGGAATGCCGAT | 41944 | 42538; 42548 |
| DX- | | | |
| 042 | | | |
| AS- | CAATTTCAGGCTTTTTCT | 30435 | |
| DX- | | | |
| 043 | | | |
| AS- | TGCCTACAGAAGGCTTTG | 31124 | |
| DX- | | | |
| 044 | | | |
| AS- | ATCTCTGCACTCATCACA | 30402 | 42276 |
| DX- | | | |
| 045 | | | |
| AS- | CTGATCACCGAAGTTCTG | 41945 | |
| DX- | | | |
| 046 | | | |
| AS- | CCAGGAGATGTAACTCTA | 30368 | 42761; 42518 |
| DX- | | | |
| 048 | | | |
| AS- | GAAAGAGAGGCCACCGCC | 30221 | 42303 |
| DX- | | | |
| 049 | | | |
| AS- | GTAGCCAGCCAGGTGTTC | 41946 | 42304; 42579; 42778; |
| DX- | | | 42490 |
| 050 | | | |
| AS- | GCCCCTCCGTAGCCAGCC | 41947 | 42305; 42265; 42762; |
| DX- | | | 42231 |
| 051 | | | |
| AS- | TGCTGTCCGAGGGTGTCG | 30092 | |
| DX- | | | |
| 052 | | | |
| AS- | AGGGGTGCTTCCAGCGAG | 30179 | 42298; 42342 |
| DX- | | | |
| 053 | | | |
| AS- | TTCTTCCTCGCTGAGGGG | 30183 | 42299; 42894 |
| DX- | | | |
| 054 | | | |
| AS- | CGGTATTCTTCCTCGCTG | 30188 | 42300; 42789 |
| DX- | | | |
| 055 | | | |
| AS- | TCCTCCAGCAGAGCCCGG | 41948 | 42301; 42588; 42140; |
| DX- | | | 42842; 42810; 42614 |
| 056 | | | |
| AS- | CCTGGGCCGGCTCTGGGA | 41949 | 42311; 42540; 42545; |
| DX- | | | 42513 |
| 057 | | | |
| AS- | TGCTGGTACCTGGGCCGG | 41950 | 42312; 42350 |
| DX- | | | |
| 058 | | | |
| AS- | TCTATAGGATCCACAGGG | 30392 | |
| DX- | | | |
| 059 | | | |
| AS | GGGCATTTTAATATATCTCTGAACT | 41951 | |
| DX- | | | |
| 060 | | | |
| AS- | TATCTTCTGAACTAATCATCCA | 41952 | |
| DX- | | | |
| 061 | | | |
| AS- | CAGGAGATGTAACTCTAATCCAG | 41953 | |
| DX- | | | |
| 062 | | | |
| AS- | CTCTCACCGGGCCTAGACCTAGAAG | 41954 | |
| DX- | | | |
| 063 | | | |
| AS- | TGCGCACTGCGCGCAGGTCTAGCCA | 41955 | |
| DX- | | | |
| 064 | | | |
| AS- | ACTGCGCGCAGGTCTAGCCAGGAAG | 41956 | |
| DX- | | | |
| 065 | | | |
| AS- | CGGGGTGCGCACTGCGCGCAGGTCT | 41957 | |
| DX- | | | |
| 066 | | | |
| AS- | TGCGCACTGCGCGCAGGTCTAGCCAG | 41958 | |
| DX- | GAAG | | |
| 067 | | | |
| AS- | ACTGCGCGCAGGTCTAGCCAGGAAGC | 41959 | |
| DX- | GGGC | | |
| 068 | | | |
| AS- | ACCCGACCCCGTCCCAACCCCGCGT | 41960 | |
| DX- | | | |
| 069 | | | |
| AS- | TGGGCTGGTGGAGAGGCAG | 41961 | 42521; 42185; 42246; |
| DX- | | | 42313; 42765; 42186; |
| 070 | | | 42658; 42434; 42505; |
| | | | 42501; 42551; 42478; |
| | | | 42320; 42821; 42585; |
| | | | 42809; 42187; 42745; |
| | | | 42356; 42456; 42247; |
| | | | 42256; 42615; 42682; |
| | | | 42668; 42451; 42835; |
| | | | 42557; 42662; 42691; |
| | | | 42537; 42820; 42861; |
| | | | 42876; 42611; 42634; |
| | | | 42748; 42530 |
| AS- | TTCCTCTCTCCATCTCTGC | 41962 | |
| DX- | | | |
| 071 | | | |
| AS- | TTGTCCCGGAGGAAACCGC | 41963 | |
| DX- | | | |
| 072 | | | |
| AS- | AATCACGCCTCCGTCGTCC | 41964 | |
| DX- | | | |
| 073 | | | |
| AS- | TTCCCTGCATGTTTCCGGGTGCCCG | 41965 | |
| DX- | | | |
| 074 | | | |
| AS- | CTTCCCTGCATGTTTCCGG | 41966 | |
| DX- | | | |
| 075 | | | |
| AS- | TGTGGCTCTCGTTCATTTC | 41967 | |
| DX- | | | |
| 076 | | | |
| AS- | CTCCGTGGGAGTCTTGAGTGTGCCA | 41968 | |
| DX- | | | |
| 077 | | | |
| AS- | TGGAACTGAACCTCCGTGG | 41969 | |
| DX- | | | |
| 078 | | | |
| AS- | TGGTGGTGGTGGTGGTGGT | 41970 | |
| DX- | | | |
| 079 | | | |
| AS- | CACCCCTTCATGAATGGCGCC | 41971 | |
| DX- | | | |
| 080 | | | |
| AS- | ACAGGCTCCACCCCTTCATG | 41972 | |
| DX- | | | |
| 081 | | | |
| AS- | TTCCGCTCAAAGCAGGCCTC | 41973 | |
| DX- | | | |
| 082 | | | |
| AS- | AAAGCGATCCTTCTCAAAGGCTCGG | 41974 | |
| DX- | | | |
| 083 | | | |
| AS- | CCTGCGCGGGCGCCCTGCCGC | 41975 | |
| DX- | | | |
| 084 | | | |
| AS- | TATCTCTGAACTAATCATC | 41976 | |
| DX- | | | |
| 085 | | | |
| AS- | AGCGCCTGGCGGCGGAACGCAGACC | 41977 | |
| DX- | | | |
| 086 | | | |
| AS- | ATCTCTGCCCGCCTTCCCTCCCGCC | 41978 | |
| DX- | | | |
| 087 | | | |
| AS- | AAACCAGATCTGAATCCTGGAC | 41979 | |
| DX- | | | |
| 088 | | | |
| AS- | TTTCTAGGAGAGGTTGCGCCTG | 41980 | |
| DX- | | | |
| 089 | | | |
| AS- | AGCGTCGGAAGGTGG | 21508 | 42228; 42428; 42495; |
| DX- | | | 42332; 42462; 42420; |
| 090 | | | 42152; 42241; 42860; |
| | | | 42674 |
| AS- | AGATCCCCTCTGCC | 41981 | |
| DX- | | | |
| 091 | | | |
| AS- | ACAGCGTCGGAAGGTG | 23943 | 42620; 42618; 42677 |
| DX- | | | |
| 094 | | | |
| AS- | GACAGCGTCGGAAGGT | 23944 | 42596 |
| DX- | | | |
| 095 | | | |
| AS- | AGACAGCGTCGGAAGG | 23945 | |
| DX- | | | |
| 096 | | | |
| AS- | CCTAGACAGCGTCGGAAGGTAG | 41982 | |
| DX- | | | |
| 097 | | | |
| AS- | CAGGAATCCCAGGCCG | 41983 | 42875; 42288; 42460; |
| DX- | | | 42572; 42608; 42833; |
| 098 | | | 42630; 42437; 42262; |
| | | | 42598; 42338; 42323 |
| AS- | CAGGAATCCCAGGCC | 21371 | 42389; 42874; 42280; |
| DX- | | | 42207; 42459; 42318; |
| 099 | | | 42637; 42327; 42607; |
| | | | 42533; 42560; 42251; |
| | | | 42799; 42881; 42629; |
| | | | 42436; 42532; 42337; |
| | | | 42553; 42739; 42322; |
| | | | 42571; 42155 |
| AS- | CGGCTCTGGGATCCCCGGGA | 41984 | 42316 |
| DX- | | | |
| 100 | | | |
| AS- | GGCTCTGGGATCCCCG | 41985 | 42655; 42291; 42453; |
| DX- | | | 42425; 42837; 42681; |
| 101 | | | 42731; 42679; 42801; |
| | | | 42823; 42335; 42603; |
| | | | 42218; 42825; 42379; |
| | | | 42604; 42610; 42742; |
| | | | 42385; 42486 |
| AS- | GGCTCTGGGATCCCC | 41986 | 42654; 42282; 42520; |
| DX- | | | 42452; 42836; 42680; |
| 102 | | | 42730; 42461; 42510; |
| | | | 42587; 42800; 42822; |
| | | | 42334; 42580; 42602; |
| | | | 42258; 42529; 42474; |
| | | | 42270; 42264; 42351; |
| | | | 42766; 42764; 42399; |
| | | | 42377; 42609; 42622; |
| | | | 42718; 42484 |
| AS- | TAGACAGCGTCGGAAGGTGG | 38049 | 42732; 42333; 42245; |
| DX- | | | 42564; 42339; 42419 |
| 104 | | | |
| AS- | GAGCTCCCTTGCACGTCAGC | 41987 | |
| DX- | | | |
| 105 | | | |
| AS- | AGCTCCCTTGCACGTC | 23896 | 42447; 42830 |
| DX- | | | |
| 106 | | | |
| AS- | GAGCTCCCTTGCACGT | 23897 | 42448; 42494; 42180; |
| DX- | | | 42388; 42150 |
| 107 | | | |
| AS- | GAGCTCCCTTGCACG | 21469 | 42445; 42782; 42852; |
| DX- | | | 42867; 42179; 42387; |
| 108 | | | 42569; 42525; 42380; |
| | | | 42383 |
| AS- | CGTAGCCAGCCAGGTGTTCC | 41988 | 42314 |
| DX- | | | |
| 109 | | | |
| AS- | TAGCCAGCCAGGTGT | 21339 | 42565; 42854; 42279; |
| DX- | | | 42819; 42528; 42358; |
| 111 | | | 42719; 42326; 42818; |
| | | | 42403; 42576; 42794; |
| | | | 42628; 42156; 42407; |
| | | | 42416; 42727; 42366; |
| | | | 42798; 42855; 42871; |
| | | | 42641; 42325; 42415; |
| | | | 42888; 42847; 42164; |
| | | | 42408; 42142; 42205; |
| | | | 42638; 42526; 42442; |
| | | | 42729; 42523; 42201; |
| | | | 42575 |
| AS- | CTTCTATAGGATCCACAGGG | 38106 | 42594 |
| DX- | | | |
| 112 | | | |
| AS- | ATGCCCAGGAAAGAATGGCA | 41989 | |
| DX- | | | |
| 113 | | | |
| AS- | CAAAGACAGACAGAGGTA | 41990 | 42704; 42768; 42240; |
| DX- | | | 42163; 42441 |
| 114 | | | |
| AS- | GTCCTAAAGCTCCTCCA | 26770 | 42294; 42623; 42336; |
| DX | | | 42803; 42431; 42583; |
| 116 | | | 42750 |
| AS- | TCCTAAAGCTCCTCCAG | 26769 | 42293; 42162; 42535; |
| DX- | | | 42780; 42467; 42430; |
| 117 | | | 42582; 42183; 42208 |
| AS- | GGGATGCCTTGCATCTG | 26720 | 42292; 42578; 42324; |
| DX | | | 42349; 42395 |
| 118 | | | |
| AS- | GAAACCAGATCTGAATC | 41991 | 42368; 42547; 42400 |
| DX- | | | |
| 119 | | | |
| AS- | GGGTCCAAACGAGTCTC | 41992 | 42542; 42268; 42747 |
| DX- | | | |
| 120 | | | |
| AS- | GCTGCAGAAACTCCGG | 41993 | 42285; 42519; 42190; |
| DX- | | | 42723; 42319; 42192; |
| 121 | | | 42616 |
| AS- | TGTTCCCCGCGAAAGA | 23783 | 42286; 42220 |
| DX- | | | |
| 122 | | | |
| AS- | GTGACATATCTCTGCA | 23998 | 42807; 42804; 42409 |
| DX- | | | |
| 123 | | | |
| AS- | CATATCTCTGCACTCA | 23994 | 42740; 42229; 42889; |
| DX- | | | 42652 |
| 124 | | | |
| AS- | GACATATCTCTGCACT | 23996 | 42373; 42487; 42139 |
| DX- | | | |
| 125 | | | |
| AS- | GGGGTCCAAACGAGTC | 41994 | 42466; 42541; 42438; |
| DX- | | | 42165; 42266; 42522 |
| 126 | | | |
| AS- | TACAGGGGATATTGTG | 41995 | 42708; 42457; 42828 |
| DX- | | | |
| 127 | | | |
| AS- | AGCAGGGCGGTCTGG | 41996 | 42814; 42211; 42515; |
| DX- | | | 42701; 42381; 42422; |
| 128 | | | 42642; 42497; 42665; |
| | | | 42839; 42639; 42859; |
| | | | 42720; 42831; 42690; |
| | | | 42433; 42702; 42375; |
| | | | 42272; 42781 |
| AS- | AGCTGCCCCGGCTTG | 41997 | 42864; 42517; 42277; |
| DX- | | | 42791; 42367; 42751; |
| 129 | | | 42237; 42612; 42393; |
| | | | 42439; 42382; 42862; |
| | | | 42458; 42887; 42863; |
| | | | 42573; 42417; 42396; |
| | | | 42667; 42543; 42405; |
| | | | 42613; 42391; 42722; |
| | | | 42670; 42372; 42170 |
| AS- | CCCAGGAAAGAAAGG | 41998 | 42659; 42815; 42281; |
| DX- | | | 42212; 42769; 42374; |
| 130 | | | 42599; 42354; 42880; |
| | | | 42632; 42700; 42200; |
| | | | 42886; 42656; 42753; |
| | | | 42857; 42488; 42759; |
| | | | 42625; 42273; 42344; |
| | | | 42816; 42204 |
| AS- | GGTGAGCCCCGGCCGG | 41999 | 42754; 42812; 42402; |
| DX- | | | 42506; 42222; 42404; |
| 131 | | | 42471; 42341; 42145; |
| | | | 42469; 42850; 42556; |
| | | | 42883; 42840; 42756; |
| | | | 42873; 42195; 42893; |
| | | | 42671 |
| AS- | GCAGACCAGGGCGCC | 42000 | 42435; 42278; 42411; |
| DX- | | | 42792; 42749; 42591; |
| 132 | | | 42397; 42737; 42199; |
| | | | 42721; 42193; 42347; |
| | | | 42870; 42346; 42552; |
| | | | 42424; 42500; 42364; |
| | | | 42259; 42146; 42624; |
| | | | 42779; 42473; 42263; |
| | | | 42738; 42360; 42355; |
| | | | 42626; 42503; 42414; |
| | | | 42217; 42475; 42890; |
| | | | 42605; 42845; 42555; |
| | | | 42834; 42440; 42788; |
| | | | 42376; 42879; 42697; |
| | | | 42249; 42786; 42261; |
| | | | 42849 |
| AS- | CCTGGGCCGGCTCTG | 42001 | 42516; 42699; 42891; |
| DX- | | | 42664; 42213; 42283; |
| 133 | | | 42574; 42239; 42398; |
| | | | 42713; 42544; 42838; |
| | | | 42224; 42511; 42514; |
| | | | 42477; 42378; 42877; |
| | | | 42882; 42706; 42496; |
| | | | 42561; 42698; 42449; |
| | | | 42787; 42635; 42512; |
| | | | 42191; 42209; 42363; |
| | | | 42361; 42683; 42869; |
| | | | 42692; 42550; 42892; |
| | | | 42232 |
| AS- | AGAAGGCAGGAATCCCAGGC | 37973 | 42315 |
| DX- | | | |
| 134 | | | |
| AS- | CGGGTGCCTGGCCCTTC | 42002 | 42772; 42795; 42509; |
| DX- | | | 42796; 42468; 42865; |
| 135 | | | 42716; 42606; 42418 |
| AS- | CCAGCTCCTCCCGGGC | 42003 | 42724; 42736; 42696; |
| DX- | | | 42695; 42177; 42498; |
| 136 | | | 42712; 42465; 42225; |
| | | | 42234; 42646; 42643; |
| | | | 42345; 42843; 42741; |
| | | | 42567; 42714; 42238; |
| | | | 42872; 42353; 42784; |
| | | | 42647; 42649; 42601; |
| | | | 42223; 42369; 42755; |
| | | | 42657; 42562; 42257; |
| | | | 42463; 42455; 42627; |
| | | | 42771; 42645; 42777; |
| | | | 42846; 42158; 42226; |
| | | | 42255; 42243; 42159; |
| | | | 42776; 42499; 42593; |
| | | | 42631; 42644; 42470; |
| | | | 42735; 42489; 42233; |
| | | | 42640; 42597; 42384; |
| | | | 42491; 42710; 42669; |
| | | | 42252; 42527; 42549; |
| | | | 42267; 42476; 42684; |
| | | | 42216; 42174; 42184; |
| | | | 42853; 42693; 42271; |
| | | | 42709 |
| AS- | TTGTGACATATCTCTGCA | 30411 | 42808; 42805; 42410; |
| DX- | | | 42844 |
| 137 | | | |
| AS- | CTCCCTTGCACGTCA | 21466 | 42444; 42160; 42317; |
| DX- | | | 42726; 42757 |
| 138 | | | |
| AS- | GCTCCCTTGCACGTCA | 23895 | 42446; 42181; 42829; |
| DX- | | | 42570 |
| 139 | | | |
| AS- | AGCTCCCTTGCACGTCA | 26881 | 42182 |
| DX- | | | |
| 140 | | | |
| AS- | CGAGCTCCCTTGCACGTCAG | 42004 | |
| DX- | | | |
| 141 | | | |
| AS- | AAGCGATCCTTCTCAA | 42005 | 42650; 42546; 42534; |
| DX- | | | 42558; 42802 |
| 142 | | | |
| AS- | {G}*{C}*c*a*t*c*g*c*g*g*g*t*a*g*{C}* | 42006 | |
| DX- | {C} | | |
| 001-1 | | | |
| AS- | Amino C6- | 42007 | |
| DX- | {G}*{C}*c*a*t*c*g*c*g*g*g*t*a*g*{C}* | | |
| 001-2 | {C} | | |
| AS- | {T}*{G}*t*c*g*g*g*a*g*g*g*c*c*a*{T}* | 42008 | |
| DX- | {C} | | |
| 002-1 | | | |
| AS- | Amino C6- | 42009 | |
| DX- | {T}{G}t*5mec*g*g*g*a*g*g*g*5mec*5mec | | |
| 002-2 | *a{T}{C} | | |
| AS- | Amino C6- | 42010 | |
| DX- | {T}mp{G}t*c*g*g*g*a*g*g*g*c*c*a{T} | | |
| 002-3 | mp{C} | | |
| AS- | Amino C6- | 42011 | |
| DX- | [T]*[G]*t*c*g*g*g*a*g*g*g*c*c*a*[T]*[C | | |
| 002-4 | | | |
| AS- | {T}*{C}*c*a*a*a*c*g*a*g*t*c*t*c*{C}* | 42012 | |
| DX- | {G} | | |
| 003-1 | | | |
| AS- | Amino C6- | 42013 | |
| DX- | {T}*{C}*c*a*a*a*c*g*a*g*t*c*t*c*{C}* | | |
| 003-2 | {G} | | |
| AS- | {G}*{A}*t*t*c*t*g*a*a*a*c*c*a*g*{A}* | 42014 | |
| DX- | {T} | | |
| 004-1 | | | |
| AS- | Amino C6- | 42015 | |
| DX- | {G}*{A}*t*t*c*t*g*a*a*a*c*c*a*g*{A}* | | |
| 004-2 | {T} | | |
| AS- | {G}*{C}*g*g*g*c*g*c*c*c*t*g*c*c*{A}* | 42016 | |
| DX- | {C} | | |
| 005-1 | | | |
| AS- | Amino C6- | 42017 | |
| DX- | {G}*{C}*g*g*g*c*g*c*c*c*t*g*c*c*{A}* | | |
| 005-2 | {C} | | |
| AS- | {T}*{C}*a*t*c*c*a*g*c*a*g*c*a*g*{G}* | 42018 | |
| DX- | {C} | | |
| 006-1 | | | |
| AS- | Amino C6- | 42019 | |
| DX- | {T}*{C}*a*t*c*c*a*g*c*a*g*c*a*g*{G}* | | |
| 006-2 | {C} | | |
| AS- | {T}*{A}*g*c*c*a*g*c*c*a*g*g*t*g*{T}* | 42020 | |
| DX. | {T} | | |
| 007-1 | | | |
| AS- | Amino C6- | 42021 | |
| DX- | {T}*{A}*g*c*c*a*g*c*c*a*g*g*t*g*{T}* | | |
| 007-2 | {T} | | |
| AS- | {T}*mA*{G}*c*c*a*g*c*c*a*g*g*t*{G}* | 42022 | |
| DX- | mU*{T} | | |
| 007-3 | | | |
| AS- | {C}*{A}*g*c*g*t*c*g*g*a*a*g*g*t*{G}* | 42023 | |
| DX- | {G} | | |
| 008-1 | | | |
| AS | Amino C6- | 42024 | |
| DX- | {C}*{A}*g*c*g*t*c*g*g*a*a*g*g*t*{G}* | | |
| 008-2 | {G} | | |
| AS- | {T}*{A}*g*a*c*a*g*c*g*t*c*g*g*a*{A}* | 42025 | |
| DX- | {G} | | |
| 009-1 | | | |
| AS- | Amino C6- | 42026 | |
| DX- | {T}*{A}*g*a*c*a*g*c*g*t*c*g*g*a*{A}* | | |
| 009-2 | {G} | | |
| AS- | {A}*{T}*a*g*g*a*t*c*c*a*c*a*g*g*{G}* | 42027 | |
| DX- | {A} | | |
| 010-1 | | | |
| AS- | Amino C6- | 42028 | |
| DX- | {A}*{T}*a*g*g*a*t*c*c*a*c*a*g*g*{G}* | | |
| 010-2 | {A} | | |
| AS- | Amino C6- | 42029 | |
| DX- | [A]*[T]*a*g*g*a*t*c*c*a*c*a*g*g*[G]* | | |
| 010-3 | [A] | | |
| AS- | {T}*{C}*t*a*t*a*g*g*a*t*c*c*a*c*{A}* | 42030 | |
| DX- | {G} | | |
| 011-1 | | | |
| AS- | Amino C6- | 42031 | |
| DX- | {T}*{C}*t*a*t*a*g*g*a*t*c*c*a*c*{A}* | | |
| 011-2 | {G} | | |
| AS- | {G}*{C}*a*c*t*a*a*t*c*a*t*c*c*a*{G}* | 42032 | |
| DX- | {G} | | |
| 012-1 | | | |
| AS- | Amino C6- | 42033 | |
| DX- | {G}*{C}*a*c*t*a*a*t*c*a*t*c*c*a*{G}* | | |
| 012-2 | {G} | | |
| AS- | {C}*{A}*{G}*c*g*t*c*g*g*a*a*g*{G}* | 42034 | |
| DX- | {T}*{G} | | |
| 014-1 | | | |
| AS- | Amino C6- | 42035 | |
| DX- | {C}*{A}*{G}*c*g*t*c*g*g*a*a*g*{G}* | | |
| 014-2 | {T}*{G} | | |
| AS- | (C)*(C)*(T)*(A)*(G)*a*c*a*g*c*g*t*c*g* | 42036 | |
| DX- | g*(A)*(A)*(G)*(G)*(T) | | |
| 015-1 | | | |
| AS- | Amino C6- | 42037 | |
| DX- | (C)*(C)*(T)*(A)*(G)*a*c*a*g*c*g*t*c*g* | | |
| 015-2 | g*(A)*(A)*(G)*(G)*(T) | | |
| AS- | {C}*{C}*t*{A}*g*a*c*a*g*c*g*t*c*g*{G} | 42038 | |
| DX- | *{A}*a*g*{G}*{T} | | |
| 015-3 | | | |
| AS- | {A}*{T}*{A}*g*g*a*t*c*c*a*c*a*g*g*g* | 42039 | |
| DX- | {A}*{G}*{G} | | |
| 018-1 | | | |
| AS- | {A}*{T}*a*g*g*a*t*c*c*a*c*a*g*g*{G}* | 42040 | |
| DX- | a*{G}*{G} | | |
| 018-2 | | | |
| AS- | {C}*{G}*{G}*c*t*c*t*g*g*g*a*t*c*c*c* | 42041 | |
| DX- | {C}*{G}*{G} | | |
| 019-1 | | | |
| AS- | {G}*{G}*{G}*g*c*g*g*a*g*a*c*a*c*g* | 42042 | |
| DX- | {C}*{C}*{C} | | |
| 021-1 | | | |
| AS- | {A}*{G}*{A}*a*g*g*c*a*g*g*a*a*t*c*c* | 42043 | |
| DX- | {C}*{A}*{G} | | |
| 022-1 | | | |
| AS- | {G}*{C}*{A}*g*g*a*a*t*c*c*c*a*g*g*c* | 42044 | |
| DX- | {C}*{G}*{G} | | |
| 023-1 | | | |
| AS- | {G}*{C}*a*{G}*g*a*a*t*c*c*c*a*g*g*c* | 42045 | |
| DX- | c*mG*{G} | | |
| 023-2 | | | |
| AS- | {G}*{C}*mA*g*g*a*a*t*c*c*c*a*g*g*c* | 42046 | |
| DX- | {C}*mG*{G} | | |
| 023-3 | | | |
| AS- | {G}*{G}*{A}*g*t*c*t*c*t*c*a*c*c*g*g* | 42047 | |
| DX- | {G}*{C}*{C} | | |
| 025-1 | | | |
| AS- | {A}*{G}*{A}*g*g*c*c*a*g*c*g*a*g*c*t* | 42048 | |
| DX- | {C}*{C}*{C} | | |
| 026-1 | | | |
| AS- | {G}*{G}*{C}*t*c*t*g*g*g*a*t*c*c*c*c* | 42049 | |
| DX- | {G}*{G}*{G} | | |
| 027-1 | | | |
| AS- | {G}*mG*c*{T}*c*t*g*g*g*a*t*c*c*c*c*g | 42050 | |
| DX- | *{G}*{G} | | |
| 027-2 | | | |
| AS- | {G}*mG*{C}*t*c*t*g*g*g*a*t*c*c*c*c*m | 42051 | |
| DX- | G*mG*{G} | | |
| 027-3 | | | |
| AS- | {G}{G}{C}*t*c*t*g*g*g*a*t*c*c*c*c*{G} | 42052 | |
| DX- | {G}{G} | | |
| 027-4 | | | |
| AS- | {C}*{A}*{G}*a*g*a*g*g*c*c*a*g*c*g*a* | 42053 | |
| DX- | (G}*{C}*{T} | | |
| 028-1 | | | |
| AS- | {G}*{A}*{C}*a*g*c*g*t*c*g*g*a*a*g*g* | 42054 | |
| DX- | {T}*{G}*{G} | | |
| 029-1 | | | |
| AS- | Amino C6- | 42055 | |
| DX- | {G}*{A}*{C}*a*g*5mec*g*t*5mec*g*g*a | | |
| 029-2 | *a*g*g*{T}*{G}*{G} | | |
| AS- | {G}*{T}*{A}*a*c*t*c*t*a*a*t*c*c*a*g* | 42056 | |
| DX- | {G}*{T}*{T} | | |
| 030-1 | | | |
| AS- | {G}*mA*{C}*a*t*t*c*a*g*c*c*a*g*{A}* | 42057 | |
| DX- | mA*{T} | | |
| 031-1 | | | |
| AS- | {A}*{C}*{A}*a*g*g*g*c*a*c*a*g*a*g*a* | 42058 | |
| DX- | {G}*{G}*{C} | | |
| 032-1 | | | |
| AS- | {G}*{A}*{G}*c*t*c*c*c*t*t*g*c*a*c*g* | 42059 | |
| DX- | {T}*{C}*{A} | | |
| 033-1 | | | |
| AS- | Amino C6- | 42060 | |
| DX- | {G}*{A}*{G}*c*t*c*c*c*t*t*g*c*a*5mec | | |
| 033-2 | *g*{T}*{C}*{A} | | |
| AS- | {G}*mA*{G}*c*t*c*c*c*t*t*g*c*a*c*g* | 42061 | |
| DX- | {T}*mC*{A} | | |
| 033-3 | | | |
| AS- | {G}*{A}*g*{C}*{T}*c*c*c*t*t*g*c*a*c* | 42062 | |
| DX- | g*t*mC*{A} | | |
| 033-4 | | | |
| AS- | {C}*{C}*{G}*t*c*c*a*a*c*c*c*c*g*c*{G} | 42063 | |
| DX- | *{T}*{C} | | |
| 034-1 | | | |
| AS- | {C}*{C}*{T}*a*a*a*g*c*t*c*c*t*c*c*a* | 42064 | |
| DX- | {G}*{C}*{A} | | |
| 035-1 | | | |
| AS- | {G}*{C}*{G}*a*g*g*c*g*g*c*c*t*c*t*t* | 42065 | |
| DX- | {C}*{C}*{G} | | |
| 036-1 | | | |
| AS- | {G}*{C}*{C}*t*c*c*a*g*c*t*c*c*c*c*c* | 42066 | |
| DX- | {G}*{G}*{G} | | |
| 037-1 | | | |
| AS- | {G}*{G}*{T}*g*t*c*g*g*g*a*g*g*g*c* | 42067 | |
| DX- | {C}*{A}*{T} | | |
| 038-1 | | | |
| AS- | {C}*{T}*{G}*g*g*c*c*a*g*c*c*g*t*t*c* | 42068 | |
| DX- | {T}*{C}*{T} | | |
| 040-1 | | | |
| AS- | {G}*{G}*{G}*c*c*a*g*c*c*g*t*t*c*t*c* | 42069 | |
| DX- | {T}*{G}*{G} | | |
| 041-1 | | | |
| AS- | {C}*{A}*a*t*t*t*c*a*g*g*c*t*t*t*{T}*t* | 42070 | |
| DX- | {C}*{T} | | |
| 043-1 | | | |
| AS- | {T}*{G}*{C}*c*t*a*c*a*g*a*a*g*g*c*t* | 42071 | |
| DX- | {T}*{T}*{G} | | |
| 044-1 | | | |
| AS- | {A}*{T}*c*t*c*t*g*c*a*c*t*c*a*t*{C}*a* | 42072 | |
| DX- | {C}*{A} | | |
| 045-1 | | | |
| AS- | {A}*mU*{C}*t*c*t*g*c*a*c*t*c*a*t*c* | 42073 | |
| DX- | {A}*mC*{A} | | |
| 045-2 | | | |
| AS- | {C}*{T}*g*a*t*c*a*c*c*g*a*a*g*t*{T}*c | 42074 | |
| DX- | *{T}*{G} | | |
| 046-1 | | | |
| AS- | {C}*{C}*a*g*g*a*g*a*t*g*t*a*a*c*{T}*c | 42075 | |
| DX- | *{T}*{A} | | |
| 048-1 | | | |
| AS- | {G}*{A}*{A}*a*g*a*g*a*g*c*c*a*c*c* | 42076 | |
| DX- | | | |
| 049-1 | | | |
| AS- | {G}*{T}*{A}*g*c*c*a*g*c*c*a*g*g*t*g* | 42077 | |
| DX- | {T}*{T}*{C} | | |
| 050-1 | | | |
| AS- | {G}*mU*{A}*g*c*c*a*g*c*c*a*g*g*t*g* | 42078 | |
| DX- | mU*{T}*{C} | | |
| 050-2 | | | |
| AS- | {G}*mU*a*g*c*c*a*g*c*c*a*g*g*t*mG*t | 42079 | |
| DX- | *{T}*{C} | | |
| 050-3 | | | |
| AS- | {G}{T}{A}*g*c*c*a*g*c*c*a*g*g*t*g*{T} | 42080 | |
| DX- | {T}{C} | | |
| 050-4 | | | |
| AS- | {G}*{C}*{C}*c*c*t*c*c*g*t*a*g*c*c*a* | 42081 | |
| DX- | {G}*{C}*{C} | | |
| 051-1 | | | |
| AS- | {T}*{G}*{C}*t*g*t*c*c*g*a*g*g*g*t*g* | 42082 | |
| DX- | {T}*{C}*{G} | | |
| 052-1 | | | |
| AS- | {A}*{G}*{G}*g*g*t*g*c*t*t*c*c*a*g*c* | 42083 | |
| DX- | {G}*{A}*{G} | | |
| 053-1 | | | |
| AS- | {T}*{T}*{C}*t*t*c*c*t*c*g*c*t*g*a*g* | 42084 | |
| DX- | {G}*{G}*{G} | | |
| 054-1 | | | |
| AS- | {C}*{G}*{G}*t*a*t*t*c*t*t*c*c*t*c*g* | 42085 | |
| DX- | {C}*{T}*{G} | | |
| 055-1 | | | |
| AS- | {T}*{C}*{C}*t*c*c*a*g*c*a*g*a*g*c*c* | 42086 | |
| DX- | {C}*{G}*{G} | | |
| 056-1 | | | |
| AS- | {C}*{C}*{T}*g*g*g*c*c*g*g*c*t*c*t*g* | 42087 | |
| DX- | {G}*{G}*{A} | | |
| 057-1 | | | |
| AS- | {T}*{G}*{C}*t*g*g*t*a*c*c*t*g*g*g*c* | 42088 | |
| DX- | {C}*{G}*{G} | | |
| 058-1 | | | |
| AS- | {T}*{C}*{T}*a*t*a*g*g*a*t*c*c*a*c*a* | 42089 | |
| DX- | {G}*{G}*{G} | | |
| 059-1 | | | |
| AS- | {T}*{C}*t*{A}*t*a*g*g*a*t*c*c*a*c*a*g | 42090 | |
| DX- | *{G}*{G} | | |
| 059-2 | | | |
| AS- | [Amino C6-] | 42091 | |
| DX- | {T}*{C}*{T}*a*t*a*g*g*a*t*c*c*a*c*a* | | |
| 059-3 | {G}*{G}*{G} | | |
| AS- | {A}*mC*{A}*g*c*g*t*c*g*g*a*a*g*{G}* | 42092 | |
| DX- | mU*{G} | | |
| 094-1 | | | |
| AS- | {C}*mA*{G}*g*a*a*t*c*c*c*a*g*g*{C}* | 42093 | |
| DX- | mC*{G} | | |
| 098-1 | | | |
| AS- | {C}*mA*{G}*g*a*a*t*c*c*c*a*g*{G}*m | 42094 | |
| DX- | C*{C} | | |
| 099-1 | | | |
| AS- | {C}*mG*g*{C}*t*c*t*g*g*g*a*t*c*c*{C} | 42095 | |
| DX- | *{C}*g*g*{G}*{A} | | |
| 100-1 | | | |
| AS- | {G}*{G}*c*t*c*t*g*g*g*a*t*c*c*mC*mC | 42096 | |
| DX- | *{G} | | |
| 101-1 | | | |
| AS- | {G}*{G}*c*t*c*t*g*g*g*a*t*c*mC*mC* | 42097 | |
| DX- | {C} | | |
| 102-1 | | | |
| AS- | {T}{A}*g*{A}*c*a*g*c*g*t*c*g*g*a*a* | 42098 | |
| DX- | {G}*{G}*t*{G}*{G} | | |
| 104-1 | | | |
| AS- | {G}*mA*{G}*c*t*c*c*c*t*t*g*c*a*c*g* | 42099 | |
| DX- | {T}*{C}*a*{G}*{C} | | |
| 105-1 | | | |
| AS- | {A}*mG*{C}*t*c*c*c*t*t*g*c*a*c*{G}* | 42100 | |
| DX- | mU*{C} | | |
| 106-1 | | | |
| AS- | {A}*{G}*c*t*c*c*c*t*t*g*c*a*c*g*{T}* | 42101 | |
| DX- | {C} | | |
| 106-2 | | | |
| AS- | {G}*mA*{G}*c*t*c*c*c*t*t*g*c*a*{C}* | 42102 | |
| DX- | mG*{T} | | |
| 107-1 | | | |
| AS- | {G}*mA*{G}*c*t*c*c*c*t*t*g*c*{A}*mC | 42103 | |
| DX- | *{G} | | |
| 108-1 | | | |
| AS- | {C}*{G}*t*{A}*g*c*c*a*g*c*c*a*g*g*{T} | 42104 | |
| DX- | *mG*t*t*{C}*{C} | | |
| 109-1 | | | |
| AS- | {T}*mA*{G}*c*c*a*g*c*c*a*g*g*{T}*m | 42105 | |
| DX- | G*{T} | | |
| 111-1 | | | |
| AS- | {C}*{T}*t*c*{T}*{A}*t*a*g*g*a*t*c*c*a | 42106 | |
| DX- | *c*a*{G}*{G}*{G} | | |
| 112-1 | | | |
| AS- | (T)*(T)*(C)*(T)*a*t*a*g*g*a*t*c*c*a*c*a | 42107 | |
| DX- | *(G)*(G)*(G)*(C) | | |
| 112-2 | | | |
| AS- | {A}*{T}*g*c*c*c*a*g*g*a*a*a*g*a*{A}* | 42108 | |
| DX- | mU*g*g*{C}*{A} | | |
| 113-1 | | | |
| AS- | {C}*mA*{A}*a*g*a*c*a*g*a*c*a*g*a*{G} | 42109 | |
| DX- | *G*{T}*{A} | | |
| 114-1 | | | |
| AS- | {G}*mU*c*{C}*t*a*a*a*g*c*t*c*c*t*mC | 42110 | |
| DX- | *{C}*{A} | | |
| 116-1 | | | |
| AS- | t*{C}*c*t*a*a*a*g*c*t*c*c*t*c*{C}*mA* | 42111 | |
| DX- | {G} | | |
| 117-1 | | | |
| AS- | {G}*{G}*g*{A}*{T}*g*c*c*t*t*g*c*a*t* | 42112 | |
| DX- | c*{T}*{G} | | |
| 118-1 | | | |
| AS- | {G}*{A}*{A}*a*c*c*a*g*a*t*c*t*g*{A}* | 42113 | |
| DX- | a*{T}*{C} | | |
| 119-1 | | | |
| AS- | {G}*{G}*{G}*t*c*c*a*a*a*c*g*a*g*{T}* | 42114 | |
| DX- | c*{T}*{C} | | |
| 120-1 | | | |
| AS- | {G}*mC*{T}*g*c*a*g*a*a*a*c*t*c*{C}* | 42115 | |
| DX- | mG*{G} | | |
| 121-1 | | | |
| AS- | {T}*mG*{T}*t*c*c*c*c*g*c*g*a*a*{A}* | 42116 | |
| DX- | {G}*{A} | | |
| 122-1 | | | |
| AS- | {G}*{T}*{G}*a*c*a*t*a*t*c*t*c*t*{G}* | 42117 | |
| DX- | {C}*{A} | | |
| 123-1 | | | |
| AS- | {C}*{A}*mU*a*t*c*t*c*t*g*c*a*c*mU* | 42118 | |
| DX- | {C}*{A} | | |
| 124-1 | | | |
| AS- | {G}*{A}*c*a*t*a*t*c*t*c*t*g*{C}*a*{C} | 42119 | |
| DX- | *{T} | | |
| 125-1 | | | |
| AS- | {G}*{G}*{G}*g*t*c*c*a*a*a*c*g*a*mG* | 42120 | |
| DX- | {T}*{C} | | |
| 126-1 | | | |
| AS- | {T}*mA*{C}*a*g*g*g*g*a*t*a*t*t*{G}* | 42121 | |
| DX- | mU*{G} | | |
| 127-1 | | | |
| AS- | {A}*mG*mC*a*g*g*c*g*g*t*c*mU*m | 42122 | |
| DX- | G*{G} | | |
| 128-1 | | | |
| AS- | {A}*mG*{C}*t*g*c*c*c*c*g*g*c*t*mU* | 42123 | |
| DX- | {G} | | |
| 129-1 | | | |
| AS- | {C}*mC*{C}*a*g*g*a*a*a*g*a*a*{A}*m | 42124 | |
| DX- | G*{G} | | |
| 130-1 | | | |
| AS- | {G}*{G}*mU*g*a*g*c*c*c*c*g*g*c*c* | 42125 | |
| DX- | {G}*{G} | | |
| 131-1 | | | |
| AS- | {G}*{C}*a*g*a*c*c*a*g*g*g*c*g*{C}* | 42126 | |
| DX- | {C} | | |
| 132-1 | | | |
| AS- | {C}*{C}*mU*g*g*g*c*c*g*g*c*t*{C}*m | 42127 | |
| DX- | U*{G} | | |
| 133-1 | | | |
| AS- | {C}*{C}*t*g*g*g*c*c*g*g*c*t*c*{T}* | 42128 | |
| DX- | {G} | | |
| 133-2 | | | |
| AS- | (A)*(G)*(A)*(A)*g*g*c*a*g*g*a*a*t*c*c* | 42129 | |
| DX- | c*(A)*(G)*(G)*(C) | | |
| 134-1 | | | |
| AS- | {A}*{G}*{A}*a*g*g*c*a*g*g*a*a*t*c*c* | 42130 | |
| DX- | c*mA*{G}*mG*{C} | | |
| 134-2 | | | |
| AS- | {C}*{G}*g*g*t*g*c*c*t*g*g*c*c*mC*t* | 42131 | |
| DX- | {T}*{C} | | |
| 135-1 | | | |
| AS- | {C}*{C}*a*g*c*t*c*c*t*c*c*c*g*g*mG* | 42132 | |
| DX- | {C} | | |
| 136-1 | | | |
| AS- | {T}*{T}*g*t*g*a*c*a*t*a*t*c*t*c*t*{G}* | 42133 | |
| DX- | mC*{A} | | |
| 137-1 | | | |
| AS- | {C}*mU*{C}*c*c*t*t*g*c*a*c*g*{T}*mC | 42134 | |
| DX- | *{A} | | |
| 138-1 | | | |
| AS- | {G}*mC{T}*c*c*c*t*t*g*c*a*c*g*{T}*m | 42135 | |
| DX- | C*{A} | | |
| 139-1 | | | |
| AS- | {A}*{G}*c*{T}*c*c*c*t*t*g*c*a*c*g*{T} | 42136 | |
| DX- | *mC*{A} | | |
| 140-1 | | | |
| AS- | (C)*(G)*(A)*(G)*c*t*c*c*c*t*t*g*c*a*c*g | 42137 | |
| DX- | *(T)*(C)*(A)*(G) | | |
| 141-1 | | | |
| AS- | {A}*mA*{G}*c*g*a*t*c*c*t*t*c*t*{C}*m | 42138 | |
| DX- | A*{A} | | |
| 142-1 | | | |

{N} can be an LNA; [N] can be a BNA; (N) can be a 2′-methyloxyethyl-modified uracil, guanine, adenine, or cytosine; * can be a phosphothionate-modified backbone; mp can be a methylphosphonate-modified backbone; Amino C6- can be 5′ amino-carbon 6 chain; 5mec can be 5-methyldeoxycytosine.

In the right most column of table 2 are RNAs that are partially complementary to listed DUX4 targeted oligonucleotides, but originating from a different genetic loci. Using a modified script of GGGenome (https://gggenome.dbcls.jp/), which allows rapid alignment of our oligonucleotide sequences to the human transcriptome (Human RNA Refseq release 205, March 2021). This script identified all transcripts that are partially complimentary to each possible oligonucleotide targeting DUX4, containing no more than 4 mismatches, bulges, insertions or deletions, containing two regions of complementarity at least 7 contiguous bases long, or one region at least 10 contiguous bases long. These interactions can also have a predicted TM of about 40° C. to about 65° C.

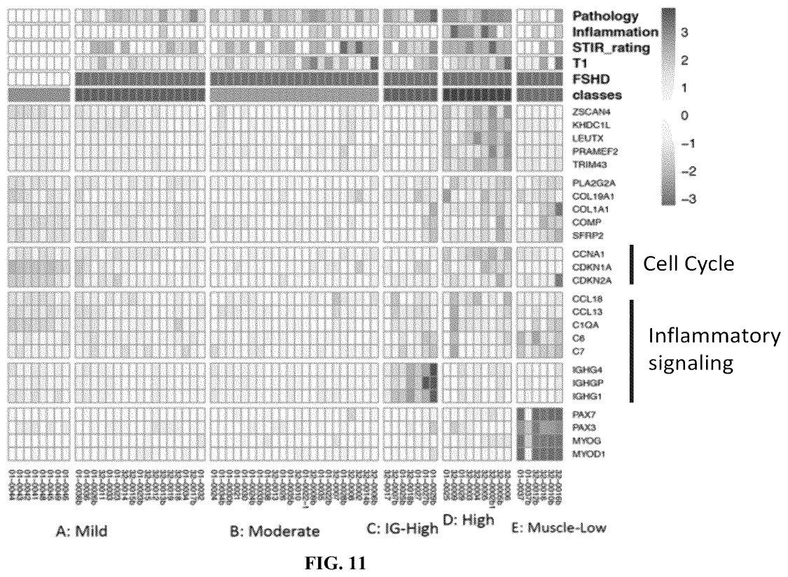

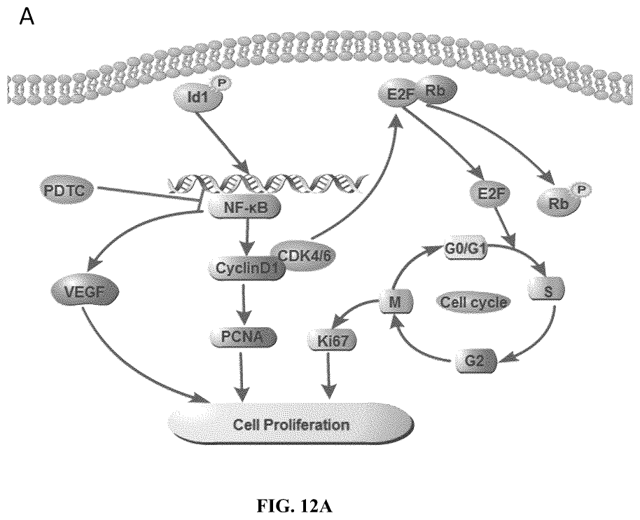

To understand what other transcripts may be related to FSHD and be beneficial to target in addition, we assembled a database of 10 studies with rigorous standards for sample handling, transcriptomic profiling by microarray and RNAseq, and significant patient information. We identified the genes that were commonly upregulated in FSHD muscle vs. control muscle among published datasets or using our own RNA-seq analysis. Interestingly, the clusters align well with clinical severity scores (i.e., mild, moderate, or severe diseases). Supporting our analysis, similar results were obtained from a similar analysis from a subset of the samples included in our larger meta-analysis as displayed in FIG. 11. From this analysis we created a database of upregulated genes in FSHD keeping with each gene the supporting evidence for this dysregulation, and any associated clinical correlations. From this database we next performed pathway enrichment analysis utilizing GO pathway analysis (12). The top upregulated pathways include inflammatory response and other immune regulated pathways, cellular proliferation, cell cycle regulation. The additional targeted RNAs represent transcripts that are upregulated or otherwise associated with the disease and may be beneficial to knockdown in addition to DUX4.

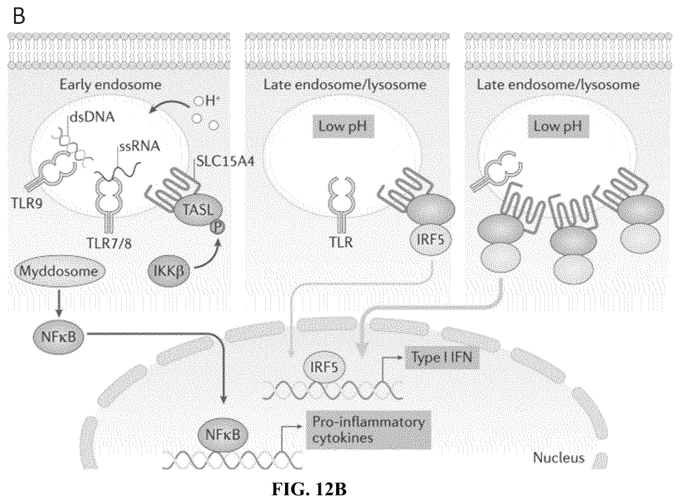

In addition, numerous RNA subsequences of additional genes associated with FSHD. For example, AS-DX-007 (SEQ. ID. NO. 23,789) is predicted to target three co-targets associated with FSHD, for example, DBET, MKI67, and IRF5. DBET is a non-coding RNA associated with opening of the D4Z4 repeats, and expression of DUX4 (38). MKI67 encodes the Ki-67 protein, which is upregulated FSHD muscle tissue, and may be involved in the DUX4 induction of the muscle fiber cell proliferation and damage. (FIG. 12A). IRF5 (Interferon Regulatory Factor 5) encodes a transcription factor that is upregulated by several inflammatory signals, and results in the expression of several cytokines such as TNF, and induction of the intracellular interferon response (FIG. 12B). Target subsequences within the transcripts of the mRNA of these genes (including those of DUX4 itself, are provided herein in Table 3 as shown in RNA form.

| Gene | | | SEQ | | | | SEQ |
|---|---|---|---|---|---|---|---|
| Name | Site | Sequence | ID NO: | Gene Name | Site | Sequence | ID NO: |
| A4GNT | 1 | AGTGCAGGATAT | 42139 | LINC00661 | 1 | CAGCCGGGGCA | 42517 |
| | | GTT | | | | GCA | |
| ABCA7 | 1 | CCTGGCTCTGCT | 42140 | LINC01275 | 1 | TACAGTCACAT | 42518 |
| | | GGAGGA | | | | CTCCTGG | |
| ABCB1 | 1 | TCCTCCTGCTGG | 42141 | LINC01602 | 1 | CCGGAGTTTCT | 42519 |
| | | ATGA | | | | GCC | |
| ABCB9 | 1 | ACCCCTGGCTGG | 42142 | LINC01605 | 1 | GGGGATCCCAT | 42520 |
| | | CA | | | | AACC | |
| ABCB9 | 2 | GCCTGCTGCTGG | 42143 | LINC01615 | 1 | CTGCCTCTCCT | 42521 |
| | | GA | | | | CCATCCAA | |
| ABCD2 | 1 | GCCTTGCTGGAT | 42144 | LINC02391 | 1 | GCCTGTTTGGA | 42522 |
| | | GA | | | | CCCC | |
| ABHD15 | 1 | CCCGCCGGGGCT | 42145 | LINC02725 | 1 | ACACCTGGCTC | 42523 |
| | | CTCC | | | | CTA | |
| ABHD2 | 1 | GGAGCCTGGTCT | 42146 | LINC02725 | 2 | AACACCTGGCT | 42524 |
| | | GC | | | | CCTA | |
| ABTB1 | 1 | GCCCTGCTGGAT | 42147 | LINC02802 | 1 | GGGGCAAGGG | 42525 |
| | | GA | | | | AGCTC | |
| ACE | 1 | CACCGCGGAGA | 42148 | LINC02904 | 1 | AACCTGGCTGG | 42526 |
| | | ATGC | | | | CTT | |
| ACOT6 | 1 | ACTGACTCCGCG | 42149 | LINGO3 | 1 | GCGCTGGAGGA | 42527 |
| | | GAG | | | | GCTGG | |
| ACOXL | 1 | ACATGAAGGGA | 42150 | LMF1 | 1 | CCACCTGGCTG | 42528 |
| | | GCTC | | | | GCAA | |
| ACP3 | 1 | GGCTGCTGCTGG | 42151 | LMX1A | 1 | AGGGATCCCAG | 42529 |
| | | ATGA | | | | AGTC | |
| ACTL10 | 1 | CCACCTTCCGAC | 42152 | LOC | 1 | CTCCCTCTCCA | 42530 |
| | | T | | 100507403 | | CCCCCA | |
| ACTL10 | 2 | CACCTTCCGACT | 42153 | LOC | 1 | TCAGCAGCTGA | 42531 |
| | | G | | 101927533 | | GGC | |
| ACTL10 | 3 | CCACCTTCCGAC | 42154 | LOXL2 | 1 | GGCCTGGGATT | 42532 |
| | | TG | | | | CG | |
| ADAM28 | 1 | AGCCTGGGATTA | 42155 | LPIN2 | 1 | GGCCTGGGATC | 42533 |
| | | CTG | | | | CCTC | |
| ADAM32 | 1 | ACACCTGGCTTC | 42156 | LPIN2 | 2 | TTGAGAAGGAT | 42534 |
| | | TA | | | | GCTT | |
| ADAM32 | 2 | AACACCTGGCTT | 42157 | LRP10 | 1 | CAGGAGGAGCT | 42535 |
| | | CTA | | | | TTAGGG | |
| ADAMTS12 | 1 | GCCCAGAGGAG | 42158 | LRP10 | 2 | TCAGGAGGAGC | 42536 |
| | | CTGG | | | | TTTAGG | |
| ADAMTS8 | 1 | GCGCGGGAGGA | 42159 | LRRC8A | 1 | CTGCCTCTCCA | 42537 |
| | | GCGGG | | | | CGCACA | |
| ADAMTSL3 | 1 | TGACGTGCAAGC | 42160 | LRRN2 | 1 | CTCGGCATTCC | 42538 |
| | | GG | | | | GAAGC | |
| ADCY3 | 1 | GCGGCCGGGCG | 42161 | LRRN2 | 2 | GGGGAGGGCG | 42539 |
| | | CCCGC | | | | CCCGC | |
| ADGRA1 | 1 | CTGGAGGAGCTT | 42162 | LRRN4 | 1 | TCCCAGACCCG | 42540 |
| | | CAGA | | | | CCCAGG | |
| ADGRA2 | 1 | GACCTCTGCTGT | 42163 | LUZP1 | 1 | GACTGTTTGGA | 42541 |
| | | CTTTG | | | | CCCA | |
| ADGRD1 | 1 | ACACCTGGCTGC | 42164 | LUZP1 | 2 | GTGACTGTTTG | 42542 |
| | | TG | | | | GACCC | |
| ADGRD1 | 2 | GCCTCTTTGGAC | 42165 | LYPD1 | 1 | CAGCCGGGGCA | 42543 |
| | | CCC | | | | GCC | |
| ADGRE5 | 1 | GCCTTCTGCTGG | 42166 | LZTS1 | 1 | CAGGCCGGCCC | 42544 |
| | | ATGA | | | | AGA | |
| ADGRL3 | 1 | GCGGCCGGGCG | 42167 | LZTS1 | 2 | TCCCAGGCCGG | 42545 |
| | | CCCGC | | | | CCCAGA | |
| ADGRL3 | 2 | GGCTGCTGCTGG | 42168 | MAB21L4 | 1 | TTGAGAAGGAT | 42546 |
| | | AGA | | | | CCTC | |
| AFAP1L2 | 1 | GAGGCCCTCCCG | 42169 | MAFB | 1 | TATTCAGACTG | 42547 |
| | | GCA | | | | GTTTC | |
| AFF3 | 1 | CAAGCCGGGGA | 42170 | MANIC1 | 1 | AACGCATTCCG | 42548 |
| | | GCG | | | | GAGC | |
| AFF3 | 2 | CCCGCGGAGAA | 42171 | MANSC4 | 1 | GCCCGGGAGGA | 42549 |
| | | CGC | | | | GCGG | |
| AJUBA | 1 | GCCTGCTGCTGG | 42172 | MAP3K12 | 1 | CTGGGCCGGCC | 42550 |
| | | ACGG | | | | CAGG | |
| ALPP | 1 | GCTGCTGCTGGA | 42173 | MAP3K12 | 2 | CTGGCTCCACC | 42551 |
| | | GA | | | | AGCCCA | |
| ANGPTL6 | 1 | GCCCGGGAGGC | 42174 | MAP3K8 | 1 | GGCGCCCTGGT | 42552 |
| | | GCGGG | | | | TCC | |
| ANKRD50 | 1 | CGCTCGTGGCCT | 42175 | MAP3K8 | 2 | TGCCTGGGATT | 42553 |
| | | CTCTG | | | | CTG | |
| ANKS1B | 1 | CCTGATGATTAG | 42176 | MARCHF4 | 1 | TCCTTCTGTGC | 42554 |
| | | TGA | | | | CCTTGT | |
| ANKS3 | 1 | GCGGGGAGGAG | 42177 | MAST3 | 1 | CGCGCCCTGGT | 42555 |
| | | CTGG | | | | CGGC | |
| ANKS3 | 2 | CTGGAGGGCGCC | 42178 | MAST3 | 2 | CCGGCCGGGGC | 42556 |
| | | CGC | | | | CGCC | |
| ANTXR2 | 1 | AGTGCAAGGGA | 42179 | MAST3 | 3 | CAGCCTCTCCC | 42557 |
| | | CTC | | | | CCAGCCCC | |
| ANTXR2 | 1 | AGTGCAAGGGA | 42180 | MAST4 | 1 | TTGAGAAGGAT | 42558 |
| | | CTC | | | | GCTA | |
| ANXA4 | 1 | TGACGTGCAAGA | 42181 | MBD2 | 1 | TCCTGCTGGTG | 42559 |
| | | GC | | | | GATGA | |
| ANXA4 | 2 | TGACGTGCAAGA | 42182 | MCEMP1 | 1 | GGCTGGGATTC | 42560 |
| | | GCT | | | | CG | |
| APLP1 | 1 | CTGGAGGAGCGT | 42183 | MCTP1 | 1 | CAGAGCCGGCG | 42561 |
| | | AGGA | | | | CGG | |
| ARAP1 | 1 | GCCCCGGAGGA | 42184 | MEFV | 1 | ACCCTGGAGGA | 42562 |
| | | GCTGC | | | | GCTGG | |
| ARHGEF34P | 1 | GTGCTCTCACCA | 42185 | MEG3 | 1 | GGGGCAGGGC | 42563 |
| | | GCCCA | | | | GCCCAC | |
| ARHGEF35 | 1 | ATGCTCTCACCA | 42186 | MEG3 | 2 | CCACCTTCCGA | 42564 |
| | | GCCCA | | | | GCCTCCA | |
| ARHGEF5 | 1 | GTGCTCTCACCA | 42187 | MEGF11 | 1 | ACTCCTGGCTG | 42565 |
| | | GCCCA | | | | GCTG | |
| ARID3C | 1 | TTTGGATTCCTG | 42188 | MEGF11 | 2 | GATGGCCCTCC | 42566 |
| | | CCTTCT | | | | CTAA | |
| ARL5C | 1 | CCCGGGGGAGC | 42189 | MFHAS1 | 1 | GCCCTGGAGGA | 42567 |
| | | GGAGC | | | | GCTGG | |
| ARSJ | 1 | CCGGAGTTTCTG | 42190 | MGAM | 1 | TTTCTGCTGAA | 42568 |
| | | CGGA | | | | TGTC | |
| ASAP1 | 1 | CAGAGCCGGCA | 42191 | MGAT1 | 1 | CGGGCAAGGG | 42569 |
| | | AGG | | | | AGCC | |
| ASB7 | 1 | CTGGAGTTTCTG | 42192 | MGAT1 | 2 | TGCGGGCAAGG | 42570 |
| | | CAC | | | | GAGC | |
| ASCL5 | 1 | GGCGCCCTGGCT | 42193 | MICALL2 | 1 | GGCCTGGGATT | 42571 |
| | | GA | | | | CTC | |
| ASF1B | 1 | CCCTCTCTGTGC | 42194 | MICALL2 | 2 | CGGCCTGGGAG | 42572 |
| | | CCTGGT | | | | CCTG | |
| ASIC2 | 1 | CCGGCCGGGGCC | 42195 | MINK1 | 1 | CAAGACGGGGC | 42573 |
| | | GCC | | | | AGCT | |
| ASIC2 | 2 | GGGCGGGCGCC | 42196 | MIR9-3HG | 1 | CAGCGCCGGCC | 42574 |
| | | CGC | | | | CAGC | |
| ASIC2 | 3 | GGGAGCTGCTGG | 42197 | MIS18A | 1 | ACACCTGGCTG | 42575 |
| | | CCCT | | | | GTTC | |
| ATG16L2 | 1 | CTCAGGGGAGCT | 42198 | MKI67 | 1 | AGACCTGGCTG | 42576 |
| | | GGAGGC | | | | GCTT | |
| ATL3 | 1 | GGGGCCTGGTCT | 42199 | MKI67 | 2 | AAGACCTGGCT | 42577 |
| | | GC | | | | GGCTT | |
| ATP2A3 | 1 | GCTTCTTTCCTG | 42200 | MKI67 | 3 | CAGTTGAAGGC | 42578 |
| | | GG | | | | ATCCC | |
| ATP8A2 | 1 | ACACCTGGCTGC | 42201 | MKI67 | 4 | GAAGACCTGGC | 42579 |
| | | TA | | | | TGGCTTC | |
| ATP8A2 | 2 | CACACCTGGCTG | 42202 | MMEL1 | 1 | AGGGATCCCAG | 42580 |
| | | CTA | | | | AGGC | |
| ATP8B2 | 1 | CCCGGGGGAGC | 42203 | MMEL1 | 2 | GCTGCTGCTGG | 42581 |
| | | GGGGC | | | | TGA | |
| ATXN7L1 | 1 | CCTTTCTGTCCT | 42204 | MMRN2 | 1 | CTGGAGGAGCT | 42582 |
| | | GGG | | | | TTAA | |
| B3GNT3 | 1 | ACCCTGGCTGGC | 42205 | MMRN2 | 2 | TGGAGGAGCTT | 42583 |
| | | CA | | | | TAAC | |
| B3GNT3 | 2 | TCCTGCTGCTGG | 42206 | MRTFA | 1 | CTGGGATTCCT | 42584 |
| | | TGA | | | | GCTTGT | |
| BARX1 | 1 | GGCCTGGGATGC | 42207 | MST1R | 1 | CTGCCTCTTCTC | 42585 |
| | | CCG | | | | AGCCCA | |
| BCAS4 | 1 | CTGGAGGAGCAT | 42208 | MTUS2 | 1 | GCCTGCTGCTG | 42586 |
| | | TAGA | | | | ATTA | |
| BCL11A | 1 | CAGGCCGGCCCA | 42209 | MUC16 | 1 | GGGGATCCCAC | 42587 |
| | | GC | | | | AGTC | |
| BOC | 1 | CACCTTCCGAAC | 42210 | MX1 | 1 | CAGGGATCTGC | 42588 |
| | | TG | | | | TGGAGGA | |
| BTG3- | 1 | GCAGCCGCCCTG | 42211 | MYEOV | 1 | ATGGCCCTCCC | 42589 |
| AS1 | | CT | | | | AACC | |
| C1orf52 | 1 | TCTCTCTTTCCTG | 42212 | MYH10 | 1 | GCGCTGCTGGA | 42590 |
| | | GG | | | | TGA | |
| C2CD4D- | 1 | CGGCGCCGGCCC | 42213 | MYO1F | 1 | GGGCCCTGGTC | 42591 |
| AS1 | | AGG | | | | TTC | |
| C2CD4D- | 2 | TCCCTGTGGATT | 42214 | MYO1F | 2 | CTGGGATACTG | 42592 |
| AS1 | | CTCT | | | | CCTTCT | |
| C9orf47 | 1 | TCCCTGTGGATT | 42215 | MYO3A | 1 | GCCCGGGAGGG | 42593 |
| | | CAT | | | | GTGG | |
| CA9 | 1 | GCCCGGGAGGC | 42216 | MYOCD | 1 | CCCTGGGTTCC | 42594 |
| | | CTGG | | | | TATAGAAG | |
| CACNA1I | 1 | GGCGCCCGGGTC | 42217 | NCCRP1 | 1 | GCTGCTGCTGG | 42595 |
| | | TGC | | | | AGGA | |
| CADPS2 | 1 | CGGGGATCCCCG | 42218 | NCOA7 | 1 | ACCTTCCGACA | 42596 |
| | | AGCA | | | | CTTC | |
| CALHM3 | 1 | GCCTGCTGCTGG | 42219 | NDN | 1 | GCCCGAGGAGC | 42597 |
| | | CTGC | | | | TGG | |
| CAMK1D | 1 | TCTTTCGCGGGG | 42220 | NDOR1 | 1 | CGGCCTGGGAG | 42598 |
| | | AAGA | | | | TCTG | |
| CAPN5 | 1 | GGGAGCTGCTGG | 42221 | NEAT1 | 1 | CCCTCCTTTCCT | 42599 |
| | | CCTCA | | | | GGG | |
| CBLB | 1 | CCAGCCGGGGCT | 42222 | NEK11 | 1 | ATCTGGTTTCA | 42600 |
| | | CCCC | | | | GAC | |
| CCDC115 | 1 | GACCTGGAGGA | 42223 | NEUROG2 | 1 | GCCCGGGAGGA | 42601 |
| | | GCTGG | | | | GCCAG | |
| CCDC160 | 1 | CGGAGCCGGCCC | 42224 | NFATC2 | 1 | GGGGTCCCAGA | 42602 |
| | | GG | | | | GCC | |
| CCDC185 | 1 | GCCCCGGAGGA | 42225 | NFATC2 | 2 | AGGGGTCCCAG | 42603 |
| | | GCTGG | | | | AGCC | |
| CCDC88C | 1 | GCTCCGGAGGA | 42226 | NHS | 1 | CGGGGATCCCG | 42604 |
| | | GCTGG | | | | GAGGC | |
| CCDC88C | 2 | TCCGGAGGAGCT | 42227 | NIBAN2 | 1 | GGCACCCTGGT | 42605 |
| | | GGAGGC | | | | CTGT | |
| CCNJL | 1 | CCACCTTCCGAG | 42228 | NINL | 1 | GGAGGGCCAGC | 42606 |
| | | CC | | | | ACCCG | |
| CD101 | 1 | TGAGTGCAGAGT | 42229 | NIPAL4 | 1 | GGCCTGGCATT | 42607 |
| | | ATC | | | | CCTG | |
| CD93 | 1 | TCCGGCTGCTGG | 42230 | NIPAL4 | 2 | GGGCCTGGCAT | 42608 |
| | | ATGA | | | | TCCTG | |
| CDC14B | 1 | GGCTGGCTCAGA | 42231 | NLRC5 | 1 | GGGGATCCCAC | 42609 |
| | | GGGGC | | | | AGCC | |
| CDCA2 | 1 | CAAGCCGGCCCA | 42232 | NLRC5 | 2 | GGGGGATCCCA | 42610 |
| | | GC | | | | CAGCC | |
| CDK5R2 | 1 | GCCCGGGCGAG | 42233 | NOD2 | 1 | CTGCCTCCCAC | 42611 |
| | | CTGG | | | | CAGTCTA | |
| CELSR3 | 1 | GCCAGGAGGAG | 42234 | NOL4L | 1 | CAACGGGGCAG | 42612 |
| | | CTGG | | | | CT | |
| CELSR3 | 2 | GTCTGCTGCTGG | 42235 | NOLC1 | 1 | GAAGCGGGGC | 42613 |
| | | ATGG | | | | AGCT | |
| CENATAC- | 1 | GCCTGCTGCTGA | 42236 | NOS3 | 1 | CAGGGCTCTGC | 42614 |
| DT | | TGC | | | | TGGAGCA | |
| CEP170B | 1 | CAAGCCGGGGA | 42237 | NOS3 | 2 | CTGCCTCTGCT | 42615 |
| | | CT | | | | CCAGCCCC | |
| CEP170B | 2 | GCCCGGGAGGA | 42238 | NOTCH3 | 1 | CCGACTTTCTG | 42616 |
| | | GCAGA | | | | CAGC | |
| CFAP157 | 1 | GAGAGCCGGCC | 42239 | NPIPA7 | 1 | CACCTTCCGAG | 42617 |
| | | CAGC | | | | TG | |
| CFAP300 | 1 | TACCTCTTCTGT | 42240 | NPIPA7 | 2 | CACCTTCCGAG | 42618 |
| | | CTTTA | | | | TGT | |
| CHD5 | 1 | CCACCTTCCGAA | 42241 | NPIPA8 | 1 | CACCTTCCGAG | 42619 |
| | | GCT | | | | TG | |
| CHD5 | 2 | CACCTTCCGAAG | 42242 | NPIPA8 | 2 | CACCTTCCGAG | 42620 |
| | | CTC | | | | TGT | |
| CHD5 | 3 | GCCCGGGAGGG | 42243 | NRP2 | 1 | GGGAGCTGTGG | 42621 |
| | | GTGG | | | | CCTCT | |
| CHD5 | 4 | CCACCTTCCGAA | 42244 | NTRK3 | 1 | GTGAATCCCAG | 42622 |
| | | GCTC | | | | AGCC | |
| CHD5 | 5 | CCACCTTCCGAA | 42245 | NUPR2 | 1 | AGGAGGAGCTT | 42623 |
| | | GCTCCTG | | | | TACGAC | |
| CHP1P2 | 1 | CTCCTGTCCACC | 42246 | NYNRIN | 1 | GGGGCCCTGGT | 42624 |
| | | AGCCCT | | | | CTGG | |
| CHRNB4 | 1 | CTGCCTCTCCAT | 42247 | NYNRIN | 2 | GCTTTCTTTCCT | 42625 |
| | | CACCA | | | | GGT | |
| CHST11 | 1 | GCCTGCTGCTGG | 42248 | OAS3 | 1 | GGCGCCCTGGC | 42626 |
| | | GTA | | | | CGC | |
| CHST15 | 1 | GGCTCCTGGTCT | 42249 | ODAD1 | 1 | GCGGGAGGAG | 42627 |
| | | GC | | | | CTGG | |
| CHST8 | 1 | CGCCGCGGAGA | 42250 | OR13J1 | 1 | ACACCTGGCTG | 42628 |
| | | ACCGC | | | | TA | |
| CIITA | 1 | GGCTGGGATTCC | 42251 | OS9 | 1 | GGCCTGGGATC | 42629 |
| | | TA | | | | CCTG | |
| CLIP2 | 1 | GCCCAGGAGGA | 42252 | OS9 | 2 | GGGCCTGGGAT | 42630 |
| | | GCTGG | | | | CCCTG | |
| CMAHP | 1 | TGCTGGAGGAGC | 42253 | OSBPL5 | 1 | GTCCAGGAGGA | 42631 |
| | | ATCAGG | | | | GCTGG | |
| CNDP2 | 1 | CCCTCTCTGAGC | 42254 | OSBPL8 | 1 | CCGTTCTTTCCT | 42632 |
| | | CCTTGT | | | | GGA | |
| CNKSR3 | 1 | GACCTGGAGGA | 42255 | OTOG | 1 | GCCTGCTGCTG | 42633 |
| | | GCTGG | | | | GGA | |
| CNTNAP1 | 1 | CTGCCTCTCCCC | 42256 | P2RX1 | 1 | CCACCCCTCCA | 42634 |
| | | CATCCTA | | | | CCAGCCCA | |
| CNTROB | 1 | GCCCTGGAGGA | 42257 | P3H1 | 1 | CAGCCCGGCCC | 42635 |
| | | GCTGT | | | | AGG | |
| COL12A1 | 1 | GGGGATCCCAG | 42258 | PABPCIL | 1 | GCATGCTGCTG | 42636 |
| | | AACA | | | | GAGA | |
| COL23A1 | 1 | GGCGCCCTGGAC | 42259 | PAK3 | 1 | GGCCTGGGATT | 42637 |
| | | GC | | | | TG | |
| COL23A1 | 2 | GCCTGCTGCTGG | 42260 | PALS2 | 1 | ACACCTGGCTG | 42638 |
| | | GTGT | | | | CTG | |
| COL4A1 | 1 | GGGCCCTGGTCT | 42261 | PAPPA | 1 | CCAGACCGCCC | 42639 |
| | | TC | | | | GGT | |
| COL4A1 | 2 | CTCCTGGGATTC | 42262 | PAQR5 | 1 | GCCCGGGAGGA | 42640 |
| | | CTG | | | | GTGT | |
| COL5A3 | 1 | GCCTCCCTGGTC | 42263 | PAQR8 | 1 | TCACCTGGCTG | 42641 |
| | | TGC | | | | GCTG | |
| COL5A3 | 2 | TGGGATCCCAGG | 42264 | PATL2 | 1 | CCACCGCCCTG | 42642 |
| | | GCC | | | | CT | |
| COL5A3 | 3 | GGCTGGCGAGG | 42265 | PCDHA2 | 1 | GACCGGGAGG | 42643 |
| | | AGGGGC | | | | AGCTGT | |
| COL6A3 | 1 | AACTCGTTTGGA | 42266 | PCDHA4 | 1 | GACCGGGAGG | 42644 |
| | | CCCT | | | | AGCTGT | |
| COL6A3 | 2 | GACCAGGAGGA | 42267 | PCDHA5 | 1 | GACCGGGAGG | 42645 |
| | | GCTGG | | | | AGCTGT | |
| COL6A3 | 3 | GAACTCGTTTGG | 42268 | PCDHB2 | 1 | GACCGGGAGG | 42646 |
| | | ACCC | | | | AGCTGT | |
| COL6A5 | 1 | CCAGAGAACGG | 42269 | PCDHB6 | 1 | GACCGGGAGG | 42647 |
| | | TGGCAC | | | | AGCTGT | |
| COL8A1 | 1 | TGGGATCCCAGG | 42270 | PCDHB6 | 2 | TGGCGGGTCTC | 42648 |
| | | CC | | | | CGCCCC | |
| CORO1A | 1 | GCCAGGGAGGA | 42271 | PCDHGA5 | 1 | GCCGGAGGAGC | 42649 |
| | | GCTGG | | | | TGG | |
| CPNE4 | 1 | CCAGACCGCCGG | 42272 | PCED1B- | 1 | GTGGAAGGATC | 42650 |
| | | CT | | AS1 | | GCTT | |
| CPNE9 | 1 | CCCTCTTTCCTG | 42273 | PCOLCE2 | 1 | GCCTGCTGCTG | 42651 |
| | | GG | | | | GCTGC | |
| CREB3L1 | 1 | GATGCCCTCCCG | 42274 | PCSK5 | 1 | GGAGTGCAGAG | 42652 |
| | | AA | | | | ATTG | |
| CRX | 1 | CTGGGTCCCTGC | 42275 | PCSK9 | 1 | TCCACCAGCTG | 42653 |
| | | CTTCT | | | | AGGC | |
| CYP4F3 | 1 | TGTGATGAGTCT | 42276 | PDCD4- | 1 | TGGGATCCCAG | 42654 |
| | | GAGAT | | AS1 | | AACC | |
| DBET | 1 | CAAGCCGGGGC | 42277 | PDCD4- | 2 | CTGGGATCCCA | 42655 |
| | | AGCT | | AS1 | | GAACC | |
| DBET | 2 | GGCGCCCTGGTC | 42278 | PDK3 | 1 | TCTTTCTTTCCT | 42656 |
| | | TGC | | | | GAG | |
| DBET | 3 | ACACCTGGCTGG | 42279 | PDZD4 | 1 | GGCCTGGAGGA | 42657 |
| | | CTA | | | | GCTGG | |
| DBET | 4 | GGCCTGGGATTC | 42280 | PEAK3 | 1 | GTCCCTCTCCA | 42658 |
| | | CTG | | | | CCAACCCA | |
| DBET | 5 | CCATTCTTTCCT | 42281 | PEG3 | 1 | GCTTCTTTCCTG | 42659 |
| | | GGG | | | | GG | |
| DBET | 6 | GGGGATCCCAG | 42282 | PFN1 | 1 | TCACGCCAGCT | 42660 |
| | | AGCC | | | | GAGGT | |
| DBET | 7 | CAGAGCCGGCCC | 42283 | PGR | 1 | ACTCCACTCCT | 42661 |
| | | AGG | | | | GGAG | |
| DBET | 8 | GCCTGCTGCTGG | 42284 | PIK3R5 | 1 | CTGCTCTCCAC | 42662 |
| | | ATGA | | | | CAGCTCT | |
| DBET | 9 | CCGGAGTTTCTG | 42285 | PIK3R5- | 1 | ATGGGATGCCT | 42663 |
| | | CAGC | | DT | | GCCTTCT | |
| DBET | 10 | TCTTTCGCGGGG | 42286 | PIK3R6 | 1 | CAGGCCGGCCC | 42664 |
| | | AACA | | | | AGT | |
| DBET | 11 | AACACCTGGCTG | 42287 | PIRT | 1 | CCAGACCGCCA | 42665 |
| | | GCTA | | | | GCT | |
| DBET | 12 | CGGCCTGGGATT | 42288 | PKHD1 | 1 | TAGCCAGCTGA | 42666 |
| | | CCTG | | | | GGC | |
| DBET | 13 | ACTCCACACCGC | 42289 | PLA2G6 | 1 | CAGCGGGGCAG | 42667 |
| | | GGAG | | | | CT | |
| DBET | 14 | CACCGCGGAGA | 42290 | PLA2G6 | 2 | CTGCCTCTGCC | 42668 |
| | | ACTGC | | | | CCAGCCCC | |
| DBET | 15 | CGGGGATCCCAG | 42291 | PLCB3 | 1 | CCCCGGGAGAG | 42669 |
| | | AGCC | | | | CTGG | |
| DBET | 16 | CAGATGCAAGG | 42292 | PLEKHA6 | 1 | CAGGTCGGGGC | 42670 |
| | | CATCCC | | | | AGCT | |
| DBET | 17 | CTGGAGGAGCTT | 42293 | PLEKHB2 | 1 | CCGGCCGGGGC | 42671 |
| | | TAGGA | | | | CCGCC | |
| DBET | 18 | TGGAGGAGCTTT | 42294 | PLEKHD1 | 1 | AGCTCCTGGCC | 42672 |
| | | AGGAC | | | | TCTCTG | |
| DBET | 19 | GGGCGTGTCTCC | 42295 | PLEKHG3 | 1 | CCAGGGGGAGC | 42673 |
| | | GCCCC | | | | TGGTGGC | |
| DBET | 20 | CCCGGGGGAGCT | 42296 | PLPP4 | 1 | ACACCTTCCGA | 42674 |
| | | GGAGGC | | | | CACT | |
| DBET | 21 | CGGAAGAGGCG | 42297 | PLPP4 | 2 | CACCTTCCGAC | 42675 |
| | | CCTCGC | | | | ACTG | |
| DBET | 22 | CTCGCTGGAAGC | 42298 | PLPP4 | 3 | ACACCTTCCGA | 42676 |
| | | ACCCCT | | | | CACTG | |
| DBET | 23 | CCCCTCAGCGAG | 42299 | PLPP4 | 4 | CACCTTCCGAC | 42677 |
| | | GAAGAA | | | | ACTGG | |
| DBET | 24 | CAGCGAGGAAG | 42300 | PLXDC2 | 1 | ACCTGCTGCTG | 42678 |
| | | AATACCG | | | | GATGA | |
| DBET | 25 | CCGGGCTCTGCT | 42301 | PMEL | 1 | CGGGGATCCCG | 42679 |
| | | GGAGGA | | | | GAGCT | |
| DBET | 26 | TGCTGGAGGAGC | 42302 | PMEPA1 | 1 | CGGAATCCCAG | 42680 |
| | | TTTAGG | | | | AGCC | |
| DBET | 27 | GGCGGTGGCCTC | 42303 | PMEPA1 | 1 | CGGAATCCCAG | 42681 |
| | | TCTTTC | | | | AGCC | |
| DBET | 28 | GAACACCTGGCT | 42304 | PML | 1 | CTGCCTCCTCC | 42682 |
| | | GGCTAC | | | | AGCCCA | |
| DBET | 29 | GGCTGGCTACGG | 42305 | POLR2H | 1 | CCGAGCCGGCC | 42683 |
| | | AGGGGC | | | | CAGG | |
| DBET | 30 | CCGGCCTGGGAT | 42306 | POLR2H | 2 | GCCCGGGAGGG | 42684 |
| | | TCCTGC | | | | GCGGG | |
| DBET | 31 | CTGGGATTCCTG | 42307 | POM121L10P | 1 | CTGGGATCCTG | 42685 |
| | | CCTTCT | | | | CCTTCA | |
| DBET | 32 | GGCCGGTGAGA | 42308 | POM121L1P | 1 | CTGGGATCCTG | 42686 |
| | | GACTCC | | | | CCTTCA | |
| DBET | 33 | CCCGGGGATCCC | 42309 | POM121L4P | 1 | CTGGGATCCTG | 42687 |
| | | AGAGCC | | | | CCTTCA | |
| DBET | 34 | CCGGGGATCCCA | 42310 | POM121L8P | 1 | CTGGGATCCTG | 42688 |
| | | GAGCCG | | | | CCTTCA | |
| DBET | 35 | TCCCAGAGCCGG | 42311 | PON1 | 1 | CTGTGGATCCT | 42689 |
| | | CCCAGG | | | | GAGA | |
| DBET | 36 | CCGGCCCAGGTA | 42312 | PPAN- | 1 | CCTGGCCGCCC | 42690 |
| | | CCAGCA | | P2RY11 | | TGCT | |
| DBET | 37 | CTGCCTCTCCAC | 42313 | PPOX | 1 | CTGTTCCACCA | 42691 |
| | | CAGCCCA | | | | GCCCA | |
| DBET | 38 | GGAACACCTGGC | 42314 | PPP1R15A | 1 | CAGGGCCGGCC | 42692 |
| | | TGGCTACG | | | | CAGG | |
| DBET | 39 | GCCTGGGATTCC | 42315 | PPP1R15A | 2 | GCCCAGGAGGA | 42693 |
| | | TGCCTTCT | | | | GCTGA | |
| DBET | 40 | TCCCGGGGATCC | 42316 | PPT2- | 1 | GCTGCTGCTGG | 42694 |
| | | CAGAGCCG | | EGFL8 | | AGGA | |
| DBH- | 1 | GGACTGCAAGG | 42317 | PRAMEF34P | 1 | GCCCTGGAGGA | 42695 |
| AS1 | | GAG | | | | GCTGC | |
| DCAKD | 1 | GGCCTGGGATAC | 42318 | PRAMEF36P | 1 | GCCCTGGAGGA | 42696 |
| | | CTT | | | | GCTGC | |
| DDR2 | 1 | ACTGAGTTTCTG | 42319 | PRDM16 | 1 | GGCGCCCTGGG | 42697 |
| | | CAGC | | | | CTGC | |
| DDR2 | 2 | GTGCCTCTCCAC | 42320 | PRKCB | 1 | CAGAGCCGGCG | 42698 |
| | | CACCGA | | | | CAGG | |
| DDX11 | 1 | TCCTGTGCTGGA | 42321 | PRKY | 1 | CCGGGCCGGCC | 42699 |
| | | TGA | | | | CAGG | |
| DENND2A | 1 | GGCCTGGGATTC | 42322 | PROK1 | 1 | GCTCTCTTTCCT | 42700 |
| | | TG | | | | GGG | |
| DENND2A | 2 | AGGCCTGGGATT | 42323 | PROSER3 | 1 | CCAGGCCGCCC | 42701 |
| | | CTG | | | | TGCT | |
| DGKI | 1 | CAGTCAAGGCAT | 42324 | PRR3 | 1 | CCGACCGCCCT | 42702 |
| | | CCC | | | | GT | |
| DGKQ | 1 | ACCCTGGCTGGC | 42325 | PRR5L | 1 | GTTCTGGCTGA | 42703 |
| | | TC | | | | AGTC | |
| DIPK2B | 1 | ACAACTGGCTGG | 42326 | PRSS23 | 1 | TAACTTGTCTG | 42704 |
| | | CTT | | | | TCTTTG | |
| DLG2 | 1 | GGCCTGGGATTC | 42327 | PRUNE2 | 1 | GTCTGCTGCTG | 42705 |
| | | AG | | | | GTGA | |
| DLGAP2 | 1 | ATTCGGCTGAAT | 42328 | PTPN9 | 1 | CGAGCCGGCCC | 42706 |
| | | GTC | | | | CGG | |
| DLX1 | 1 | CGGAGCTCGCGG | 42329 | PTPRN2 | 1 | CTCGCGGAGAA | 42707 |
| | | CCTCT | | | | CGGC | |
| DLX1 | 2 | AGCTCGCGGCCT | 42330 | PYHIN1 | 1 | CACAATATCCC | 42708 |
| | | CTTTG | | | | CTGTG | |
| DLX4 | 1 | ATCTGGTTTCAG | 42331 | RAB31 | 1 | GCCCGGGAGGA | 42709 |
| | | AAC | | | | GCCGG | |
| DNAAF11 | 1 | CCACCTTCCGTC | 42332 | RAB37 | 1 | ACCAGGGAGG | 42710 |
| | | CT | | | | AGCTGG | |
| DNAH1 | 1 | CCACCTTCAAGC | 42333 | RAB39A | 1 | GGGCGGGCGCC | 42711 |
| | | TGTCTT | | | | CGC | |
| DNAH10 | 1 | GGGGATCCCAG | 42334 | RAPGEF4 | 1 | GTCCGGGAGGA | 42712 |
| | | GGCC | | | | GCGGG | |
| DNAH10 | 2 | TGGGGATCCCAG | 42335 | RASAL3 | 1 | CGGGCCGGCCC | 42713 |
| | | GGCC | | | | AGG | |
| DNAH14 | 1 | TGGAGGAGCTTT | 42336 | RASAL3 | 2 | GCGCTGGAGGA | 42714 |
| | | AAAC | | | | GCTGG | |
| DNAH2 | 1 | CGCCTGGGATTC | 42337 | RASGRP2 | 1 | GGGCGTGCCCG | 42715 |
| | | TG | | | | CCCC | |
| DNAH2 | 1 | CGCCTGGGATTC | 42338 | RASSF1 | 1 | GAAGGGCCGCA | 42716 |
| | | TG | | | | CCCG | |
| DNAH2 | 2 | CCACACCTACGC | 42339 | RASSF1 | 2 | GTGCGTGTCCC | 42717 |
| | | TGTCTA | | | | CGCCCC | |
| DOCK11 | 1 | AGCTCACTGGCC | 42340 | RBP1 | 1 | GGCGGTCCCAG | 42718 |
| | | TCTCAG | | | | AGCC | |
| DPF1 | 1 | CCGGCCGGGGCT | 42341 | RCVRN | 1 | CCACCTGGCTG | 42719 |
| | | CAGC | | | | GCTG | |
| DPP9- | 1 | CTCGCTGGAAGC | 42342 | RELB | 1 | CCAGACCGCCG | 42720 |
| AS1 | | CCCCT | | | | GCT | |
| DSCAM | 1 | CACCGCGGAGA | 42343 | RELL2 | 1 | GGCGCCCTGGC | 42721 |
| L1 | | ACGC | | | | CCGC | |
| DZIP1L | 1 | TCTTCCTTTCCTG | 42344 | RGPD6 | 1 | CAAGCCGGGGA | 42722 |
| | | GG | | | | GCG | |
| ECEL1 | 1 | GCCTGGAGGAG | 42345 | RGS11 | 1 | CCTGAGTTTCT | 42723 |
| | | CTGG | | | | GCGGC | |
| EDNRB | 1 | CGCGCCCTGGTT | 42346 | RHCG | 1 | GCCCAGAGGAG | 42724 |
| | | GC | | | | CTGG | |
| EFCC1 | 1 | GGCGCCCTGGCT | 42347 | RHPN2 | 1 | ACCTGCTGCTG | 42725 |
| | | GC | | | | GAGA | |
| EFCC1 | 2 | CCTGGGGGAGCT | 42348 | RIMS4 | 1 | TGCATGCAAGG | 42726 |
| | | GGAGGC | | | | GAG | |
| EGFL6 | 1 | CAGTGCAAGGC | 42349 | RIPK4 | 1 | AGACCTGGCTG | 42727 |
| | | ATCAC | | | | GCCA | |
| EGR1 | 1 | CCGGCCCAGGTC | 42350 | RIPK4 | 2 | AAGACCTGGCT | 42728 |
| | | AGCA | | | | GGCCA | |
| ELF3 | 1 | GGGGATCCCAA | 42351 | RNASE10 | 1 | AACCTGGCTGG | 42729 |
| | | GCA | | | | CCA | |
| ENC1 | 1 | GCTGCTGCTGGA | 42352 | RNF212 | 1 | GGCGGTCCCAG | 42730 |
| | | GA | | | | AGCC | |
| ENOX1 | 1 | GCCCGGGAGGA | 42353 | RNF212 | 2 | CGGCGGTCCCA | 42731 |
| | | GCGG | | | | GAGCC | |
| EPHA2 | 1 | CCTTGCTTTCCT | 42354 | RPS2P32 | 1 | CCACCTTGGAT | 42732 |
| | | GGG | | | | GCTGTCTC | |
| EPHB4 | 1 | GGCGCCCTGGAC | 42355 | RTEL1 | 1 | GCTGCTGCTGG | 42733 |
| | | TCC | | | | AGA | |
| EPHB4 | 2 | CTCCCTCCACCA | 42356 | RTEL1- | 1 | GCTGCTGCTGG | 42734 |
| | | GCTCA | | TNFRSF6B | | AGA | |
| EPSTI1 | 1 | TCCGCCAGCTGA | 42357 | RTN1 | 1 | GCCCTGAGGAG | 42735 |
| | | AGC | | | | CTGG | |
| ERICH3 | 1 | ACACCTGGCTGG | 42358 | RTN4RL2 | 1 | GCCCTGGAGGA | 42736 |
| | | GTA | | | | GCTGG | |
| ERICH3 | 2 | AACACCTGGCTG | 42359 | RUSC1 | 1 | GGACCCTGGTC | 42737 |
| | | GGTA | | | | TGC | |
| ESPN | 1 | GGCGCCCTGGCA | 42360 | S1PR5 | 1 | TGCGCCTGGTC | 42738 |
| | | GC | | | | TGC | |
| EVC2 | 1 | CCGGGCCGGCCC | 42361 | SAMD3 | 1 | GGCAGGGATTC | 42739 |
| | | AGG | | | | CTG | |
| EXOC3L1 | 1 | GCTGCTGCTGGC | 42362 | SASH1 | 1 | TGGTGCAGAGA | 42740 |
| | | TGA | | | | TACG | |
| EXOC3L2 | 1 | CGAGCCGGCCCG | 42363 | SBNO2 | 1 | GCCCGAGAGGA | 42741 |
| | | GG | | | | GCTGG | |
| EZR | 1 | GGCGCCCTGGTT | 42364 | SCN5A | 1 | CGGGGATCCAG | 42742 |
| | | TGT | | | | AGCC | |
| EZR | 2 | GCTGCTGCTGGA | 42365 | SCN5A | 2 | CCCGGGGATCC | 42743 |
| | | TA | | | | AGAGCC | |
| F11R | 1 | CCACCTGGCTGG | 42366 | SCN5A | 3 | CCGGGGATCCA | 42744 |
| | | CA | | | | GAGCCC | |
| FAM171A2 | 1 | CCAGGCGGGGC | 42367 | SDC3 | 1 | CTGGCCCCACC | 42745 |
| | | AGCT | | | | AGCCCA | |
| FAM205A | 1 | GATTCAGATGGT | 42368 | SDK1-AS1 | 1 | CCTGGATAATT | 42746 |
| | | TTC | | | | AGTGC | |
| FAM83D | 1 | GCTCTGGAGGAG | 42369 | SDK1-AS1 | 2 | GAGACTCTTTT | 42747 |
| | | CTGG | | | | GGACCA | |
| FAM83G | 1 | AGAAACGCTGG | 42370 | SEMA4C | 1 | GTCCCTCTCCC | 42748 |
| | | CCCAG | | | | CAGCCCA | |
| FANCB | 1 | AGCTCGCGGGCT | 42371 | SEMA5B | 1 | GTCGCCCTGGT | 42749 |
| | | CTCTG | | | | CTGA | |
| FAS | 1 | CAGGCGGGGCA | 42372 | SERPINE1 | 1 | TGGAGGACCTT | 42750 |
| | | GCT | | | | TAGGTC | |
| FAT1 | 1 | AGTGCAGAGATT | 42373 | SEZ6 | 1 | CAGCGGGGCAG | 42751 |
| | | GC | | | | CT | |
| FAT2 | 1 | CCTGCTTTCCTG | 42374 | SH3TC1 | 1 | GCCTGCTGCTG | 42752 |
| | | GG | | | | GGA | |
| FAT3 | 1 | CCGCCGCCCTGC | 42375 | SHANK1 | 1 | TCTGTCTTTCCT | 42753 |
| | | T | | | | GGG | |
| FAT3 | 2 | GGCGCCCTGGTG | 42376 | SHE | 1 | CCGGCCGGGGC | 42754 |
| | | C | | | | CCCC | |
| FBLIM1 | 1 | GCGGATCCCAGA | 42377 | SHISA7 | 1 | GCCCAGAGGAG | 42755 |
| | | GCC | | | | CTGG | |
| FBLIM1 | 2 | CAAGCCGGCCCA | 42378 | SHROOM1 | 1 | CCGGCCGGGGT | 42756 |
| | | GC | | | | CCCC | |
| FBLIM1 | 3 | GGCGGATCCCAG | 42379 | SIGLEC5 | 1 | TGCTGCAAGGG | 42757 |
| | | AGCC | | | | AG | |
| FBXO24 | 1 | CGGAAGGGAGC | 42380 | SIRPB1 | 1 | GCCTGCTGCTG | 42758 |
| | | TC | | | | GAAA | |
| FBXO41 | 1 | CCCGCCGCCCTG | 42381 | SKI | 1 | CCTCTTTCCTG | 42759 |
| | | CT | | | | GG | |
| FCGBP | 1 | CAAGCCGGGGC | 42382 | SKI | 2 | CCTCCCTGTGG | 42760 |
| | | AGGT | | | | GTCCGAT | |
| FER1L6 | 1 | CTGCAAGGGAG | 42383 | SLA | 1 | GAGAGTTACAT | 42761 |
| | | CC | | | | CCCTGG | |
| FGD5 | 1 | GCCCGGGAGGA | 42384 | SLC12A3 | 1 | GGCTGGCAGGG | 42762 |
| | | GCTGA | | | | AGGGGC | |
| FGF2 | 1 | CGGGGATCCCGG | 42385 | SLC18B1 | 1 | CCCGGGGACCC | 42763 |
| | | CC | | | | AGAGTC | |
| FKBP9P1 | 1 | CCGGCCTGATTC | 42386 | SLC26A8 | 1 | TGGGATCCCAG | 42764 |
| | | CTGC | | | | CGCC | |
| FKBPL | 1 | CGTGCAAGGGG | 42387 | SLC28A3 | 1 | CTCCCTCCCCA | 42765 |
| | | CAC | | | | CCAGCCCC | |
| FKBPL | 2 | ACGTGCAAGGG | 42388 | SLC36A1 | 1 | GAGGATCCCAG | 42766 |
| | | GCAC | | | | ACCC | |
| FLG- | 1 | GGCCTGGGATTT | 42389 | SLC39A11 | 1 | GCCTGCTGCTG | 42767 |
| AS1 | | G | | | | ATCA | |
| FLI1 | 1 | GGGCAGGGCGC | 42390 | SLC41A2 | 1 | TACGTCTGTCT | 42768 |
| | | TCGC | | | | GTCTTTG | |
| FLNB | 1 | CAAGCCGGGGG | 42391 | SLC46A2 | 1 | GCTTTCTTTCCT | 42769 |
| | | CT | | | | GAG | |
| FLNB | 2 | TCACTGTGGATC | 42392 | SLC7A14 | 1 | ATTCTGGCTGA | 42770 |
| | | CTAA | | | | CTGTG | |
| FLRT1 | 1 | CAACCGGGGCA | 42393 | SLC8A2 | 1 | TCCCGGGAGGA | 42771 |
| | | GCA | | | | GCGG | |
| FOSL2 | 1 | ATGGCCCTCCCA | 42394 | SLC8B1 | 1 | TAAGGGCCAGG | 42772 |
| | | AGACC | | | | CCCCG | |
| FOXF1 | 1 | GAGCTGCAAGG | 42395 | SLC9B2 | 1 | GGGAGCTCGCT | 42773 |
| | | CATCCC | | | | GGTCCT | |
| FRAT2 | 1 | CAAGCCGGGGC | 42396 | SLCO1C1 | 1 | CCTGGATGATT | 42774 |
| | | ACG | | | | TTGC | |
| FRMD8 | 1 | GGCGCCCTGGTG | 42397 | SMAP2 | 1 | ACCTGCTGCTG | 42775 |
| | | TGC | | | | GAGGA | |
| FRMD8 | 2 | CAAGCCGGCCCA | 42398 | SMPD3 | 1 | GCCGGGAGGA | 42776 |
| | | AG | | | | GCGGG | |
| GAL3ST1 | 1 | GAGGGTCCCAG | 42399 | SORCS3 | 1 | GGCCGGAGGA | 42777 |
| | | AGCC | | | | GCTGG | |
| GALNS | 1 | CATTCAGATTGG | 42400 | SORCS3 | 2 | GAAATCTGGCT | 42778 |
| | | TTTC | | | | GGCTAC | |
| GANC | 1 | TTTCTGGCTGAA | 42401 | SOWAHD | 1 | GGCGCCCTGGG | 42779 |
| | | TGCC | | | | TGC | |
| GAS6- | 1 | CCGGCCGGGGCC | 42402 | SOX17 | 1 | CTGGAGGAGCT | 42780 |
| AS1 | | CC | | | | AAGGA | |
| GAS7 | 1 | CCACCTGGCTGG | 42403 | SOX4 | 1 | CCGCCGCCCTG | 42781 |
| | | CA | | | | CT | |
| GASK1A | 1 | CCGGCCGGGGC | 42404 | SPATA6L | 1 | CTCAAGGGAGC | 42782 |
| | | ACC | | | | TC | |
| GASK1B | 1 | CAAGCCGGGGC | 42405 | SPATCI | 1 | AGCCACTCCGC | 42783 |
| | | AGCT | | | | GGAG | |
| GASK1B | 2 | GTGGCAGGGCTC | 42406 | SPOCD1 | 1 | GACCAGGAGG | 42784 |
| | | CGGC | | | | AGCTGG | |
| GCK | 1 | ACACCTGGCTGG | 42407 | SPOCD1 | 2 | CCAGGAGGAGC | 42785 |
| | | A | | | | TGGAGGC | |
| GCKR | 1 | ACACCTGGCTGC | 42408 | SPTBN2 | 1 | GGCCCCTGGTC | 42786 |
| | | A | | | | TGC | |
| GDF11 | 1 | TGCAGAGAATGT | 42409 | SPTBN2 | 2 | GAGAGCCGGCC | 42787 |
| | | CAC | | | | CAGC | |
| GDF11 | 2 | TGCAGAGAATGT | 42410 | SRGAP2 | 1 | GGCGCCCTGGC | 42788 |
| | | CACAG | | | | TTC | |
| GFOD2 | 1 | GGCGCCCTGGAC | 42411 | SRP19 | 1 | CAGCGAGGAA | 42789 |
| | | TCC | | | | GAAACCT | |
| GJA1 | 1 | TCCCTGTGTATC | 42412 | SSBP4 | 1 | GGGCAGGGCGC | 42790 |
| | | CTAT | | | | CGGC | |
| GJA3 | 1 | CCCTGCTGCTGG | 42413 | SSPOP | 1 | CCAGCCGGGGC | 42791 |
| | | ATGG | | | | AGCT | |
| GJD4 | 1 | GCCGACCTGGTC | 42414 | SSPOP | 2 | GGCCCCCTGGT | 42792 |
| | | TGC | | | | CTGT | |
| GLB1 | 1 | ATACTGGCTGGC | 42415 | SSPOP | 3 | CCCGGGGGAGC | 42793 |
| | | TA | | | | TGGGGC | |
| GNAI3 | 1 | GCACCTGGCTGG | 42416 | SSTR2 | 1 | ACACCTGGCTT | 42794 |
| | | CAA | | | | CTA | |
| GNAO1 | 1 | CAAGCCGGGGA | 42417 | ST20-AS1 | 1 | GCAGGGCCAGG | 42795 |
| | | GCC | | | | CCCCG | |
| GNAZ | 1 | CAAGGGCAGGC | 42418 | STAC2 | 1 | GAAGGGCCAG | 42796 |
| | | ACCCG | | | | GACCAG | |
| GNAZ | 2 | CCACCTTGAGCT | 42419 | STARD5 | 1 | TGACGCCAGCT | 42797 |
| | | GTCTC | | | | GATGC | |
| GPC2 | 1 | CCACCTTCCGAG | 42420 | STARD9 | 1 | AGACCTGGCTG | 42798 |
| | | GCC | | | | GCCA | |
| GPC6 | 1 | TCGGCCTAGGAT | 42421 | STARD9 | 2 | GGCCTGGGATG | 42799 |
| | | TCCTGC | | | | CTG | |
| GPR146 | 1 | CCACCGCCCTGC | 42422 | STRC | 1 | TGGGATCCCAG | 42800 |
| | | T | | | | ACC | |
| GPR146 | 2 | ACTCCACTCCGA | 42423 | STRC | 2 | CTGGGATCCCA | 42801 |
| | | GAG | | | | GACC | |
| GPR150 | 1 | AGCGCCCTGGTC | 42424 | STX2 | 1 | GTGGAAGGATC | 42802 |
| | | GGC | | | | GCTT | |
| GPR158 | 1 | CGGAGATCCCAG | 42425 | SV2B | 1 | GGGAGGAGCTT | 42803 |
| | | AGAC | | | | AGGAC | |
| GPR160 | 1 | GTGGAGGGCGC | 42426 | SYNDIGIL | 1 | TGCAGAGAATG | 42804 |
| | | CCGG | | | | TCAC | |
| GPR37 | 1 | TCCGCCAGCTGA | 42427 | SYNDIGIL | 2 | TGCAGAGAATG | 42805 |
| | | GC | | | | TCACCA | |
| GPX2 | 1 | GCCCTTCCGACG | 42428 | SYNE3 | 1 | TCCCATGGATC | 42806 |
| | | CT | | | | CTAT | |
| GPX2 | 2 | CCCTTCCGACGC | 42429 | SYNGAP1 | 1 | TGCAGAGTATG | 42807 |
| | | TA | | | | TCAC | |
| GRHL1 | 1 | CTGGGAAGCTTT | 42430 | SYNGAP1 | 2 | TGCAGAGTATG | 42808 |
| | | AGGA | | | | TCACCA | |
| GRHL1 | 2 | TGGGAAGCTTTA | 42431 | SYNGR3 | 1 | CTGCCTCTCCA | 42809 |
| | | GGAC | | | | CCTGCACC | |
| GRIA1 | 1 | TCACTGTGGATC | 42432 | TAF5 | 1 | CCGGGCTGCTG | 42810 |
| | | CAT | | | | GAGGA | |
| H2BC8 | 1 | CCAGACCGCCGT | 42433 | TAL1 | 1 | AGCCGCTGGCC | 42811 |
| | | GCG | | | | TCTCTC | |
| HAL | 1 | CTCCCTCTCCAC | 42434 | TAPT1- | 1 | CCGGCCGGGGC | 42812 |
| | | CAGCGCA | | AS1 | | ACC | |
| HECW2- | 1 | AGGCCCTGGTCT | 42435 | TBC1D32 | 1 | TCCAGTGGATC | 42813 |
| AS1 | | GC | | | | CTAT | |
| HHAT | 1 | GTCCTGGGATTC | 42436 | TCAM1P | 1 | CCAGACCTCCC | 42814 |
| | | CTG | | | | TGCT | |
| HHAT | 2 | TGTCCTGGGATT | 42437 | TCAMIP | 2 | CCTTCCTTTCCT | 42815 |
| | | CCTG | | | | GGG | |
| HIP1R | 1 | GACGTTTGGACC | 42438 | TCF7 | 1 | CCTTTCTTTCCT | 42816 |
| | | CC | | | | TGG | |
| HMX1 | 1 | CCACCCGGGGCA | 42439 | TEAD2 | 1 | ATGGCAGGGCG | 42817 |
| | | GCT | | | | CCCC | |
| HOMER3 | 1 | GGAGCCCTGGTC | 42440 | TEKT4 | 1 | GCAGCTGGCTG | 42818 |
| | | TCC | | | | GCTA | |
| HOXC11 | 1 | TCCTCTTTCTGTC | 42441 | TEKT4P2 | 1 | GCAGCTGGCTG | 42819 |
| | | TTTG | | | | GCTA | |
| HPGD | 1 | AAACCTGGCTGG | 42442 | TEPSIN | 1 | CTTCCTCTCCA | 42820 |
| | | CA | | | | CCATCCA | |
| HPGD | 2 | AAAACCTGGCTG | 42443 | TESK2 | 1 | CTGCCTCCCAC | 42821 |
| | | GCA | | | | CAGACCC | |
| HRAT92 | 1 | GGACGTGCAAG | 42444 | TESMIN | 1 | CGGGATCCCAG | 42822 |
| | | GGG | | | | AGCT | |
| HRAT92 | 2 | CGTGCAAGGGG | 42445 | TESMIN | 2 | CCGGGATCCCA | 42823 |
| | | CGC | | | | GAGCT | |
| HRAT92 | 3 | GGACGTGCAAG | 42446 | TESMIN | 3 | CCCCGGGATCC | 42824 |
| | | GGGC | | | | CAGAGCT | |
| HRAT92 | 4 | GACGTGCAAGG | 42447 | TFAP2B | 1 | CGGGGATCCAG | 42825 |
| | | GGCG | | | | AGCT | |
| HRAT92 | 5 | ACGTGCAAGGG | 42448 | TFAP2B | 2 | CCGGGGATCCA | 42826 |
| | | GCGC | | | | GAGCTG | |
| HS6ST2 | 1 | CGAGCCGGCCCG | 42449 | TFR2 | 1 | GCCTGCTGCTG | 42827 |
| | | GG | | | | GTGC | |
| HSF2BP | 1 | GTTCTGGCTGAA | 42450 | TGFB2 | 1 | GACAGTATCCC | 42828 |
| | | GTC | | | | CTGTA | |
| HTR3C | 1 | CTCCACTGCACC | 42451 | TGFBR3 | 1 | AGAGGTGCAAG | 42829 |
| | | AGCCCA | | | | GGAGC | |
| ICAM2 | 1 | CGGGATCCCAGA | 42452 | TGFBR3 | 2 | GAGGTGCAAGG | 42830 |
| | | GCT | | | | GAGCG | |
| ICAM2 | 2 | CCGGGATCCCAG | 42453 | TGM5 | 1 | CCAGACCGCCC | 42831 |
| | | AGCT | | | | AGCT | |
| ICAM2 | 3 | CCCGGGATCCCA | 42454 | TIE1 | 1 | CCCTGCTGCTG | 42832 |
| | | GAGCT | | | | GAGA | |
| IFFO1 | 1 | GCCAGGAGGAG | 42455 | TINAGL1 | 1 | CGGCCTGGGAT | 42833 |
| | | CTGG | | | | CCAG | |
| IFFO2 | 1 | CTGCCTCTCCAC | 42456 | TLE3 | 1 | GGCGCCCTGGG | 42834 |
| | | CACA | | | | CAGC | |
| IFI16 | 1 | CACAATATCCCC | 42457 | TLNRD1 | 1 | GTGTCTCTCCA | 42835 |
| | | TGTG | | | | CCAGCCCC | |
| IGFBP7 | 1 | AAAGCCGGGGC | 42458 | TMEM106A | 1 | TGGGATCCCAG | 42836 |
| | | AGCA | | | | ACC | |
| IGFBPL1 | 1 | GGCCTGGGATTC | 42459 | TMEM106A | 2 | CTGGGATCCCA | 42837 |
| | | TG | | | | GACC | |
| IGFBPL1 | 2 | TGGCCTGGGATT | 42460 | TMEM165 | 1 | CCGAGCCGGCC | 42838 |
| | | CTG | | | | CGGG | |
| IGSF10 | 1 | GGGGATCCCAA | 42461 | TMPRSS13 | 1 | CCAGACCCCCT | 42839 |
| | | ACC | | | | GCT | |
| IGSF3 | 1 | CCACCTTCCGCC | 42462 | TNFRSF12A | 1 | CCAGCCGGGGC | 42840 |
| | | T | | | | TCGCC | |
| IGSF8 | 1 | GCCCGGGAGGT | 42463 | TNFRSF21 | 1 | CCTGGATGATT | 42841 |
| | | GCTGG | | | | GTGC | |
| IKZF1 | 1 | GTGGCAGGGCG | 42464 | TNKS1BP1 | 1 | CAGGGCCTGCT | 42842 |
| | | CGCGC | | | | GGAGGA | |
| ILIR1 | 1 | GGCCGGGAGGA | 42465 | TOR4A | 1 | GCCGCGAGGAG | 42843 |
| | | GCCGG | | | | CTGG | |
| ILDR2 | 1 | GTCTGTTTGGAC | 42466 | TPK1 | 1 | TGCAGTGATAT | 42844 |
| | | CCC | | | | GTCACAA | |
| INAVA | 1 | CTGGAGGAGCTG | 42467 | TPSD1 | 1 | GGCCCCTGGTC | 42845 |
| | | AGGA | | | | TGC | |
| INKA2 | 1 | GAAGGGCCAGG | 42468 | TRAF1 | 1 | GCCAGGAGGA | 42846 |
| | | CAGCAG | | | | GCTGG | |
| INSRR | 1 | CCGCGGGGCTCA | 42469 | TRAF5 | 1 | ACACCTGGCTG | 42847 |
| | | CC | | | | TA | |
| INSRR | 2 | GTCCTGGAGGAG | 42470 | TRAF5 | 2 | AACACCTGGCT | 42848 |
| | | CTGG | | | | GTA | |
| INSYN1 | 1 | CCGGCCGGGGCC | 42471 | TRERF1 | 1 | AGCACCCTGGT | 42849 |
| | | CCC | | | | CTGC | |
| IQGAP3 | 1 | CCCGGTGGAGCT | 42472 | TRIM56 | 1 | CCGGCCGGGGC | 42850 |
| | | GGAGGA | | | | TCAGC | |
| IRS2 | 1 | GGCGCCCTGGGC | 42473 | TRIM56 | 2 | CCTGGTGGAGC | 42851 |
| | | GGC | | | | TGGAGGC | |
| ITGA6 | 1 | GGTGCTCCCAGA | 42474 | TRIM62 | 1 | CTGCAAGGGAG | 42852 |
| | | GCC | | | | TC | |
| ITGAL | 1 | GGCGCCCTGGTT | 42475 | TRIM67 | 1 | GCCCGGGAGGC | 42853 |
| | | TTC | | | | GCGGG | |
| ITGAX | 1 | GTCCAGGAGGA | 42476 | TSPAN10 | 1 | AGCCTGGCTGG | 42854 |
| | | GCTGG | | | | CTA | |
| ITIH5 | 1 | CAGAGCCGGCTC | 42477 | TSPAN13 | 1 | ACACCTGTCTG | 42855 |
| | | AGA | | | | GCTA | |
| ITIH5 | 2 | CTGCCTCTCCCC | 42478 | TSPAN13 | 2 | GACACCTGTCT | 42856 |
| | | ACCCT | | | | GGCTA | |
| KANK4 | 1 | GCTGCTGCTGGA | 42479 | TSPAN14 | 1 | CCTTTCTTTCCC | 42857 |
| | | GA | | | | AGG | |
| KCNC3 | 1 | GCCTGCTGCTGG | 42480 | TTC22 | 1 | CCAGCCAGCTG | 42858 |
| | | ATGA | | | | AGGC | |
| KCNC4 | 1 | CCCTGCTGCTGG | 42481 | TTC34 | 1 | CGAGCCGCCCT | 42859 |
| | | ATGA | | | | GCT | |
| KCNF1 | 1 | GAAGGCCCTCCC | 42482 | TTC6 | 1 | CCACCTTCCGA | 42860 |
| | | GGCA | | | | GCG | |
| KCNF1 | 2 | AAGGCCCTCCCG | 42483 | TTLL10- | 1 | CAGCCTCTCCA | 42861 |
| | | GCACC | | AS1 | | CCTGCACA | |
| KCNG2 | 1 | GGGAATCCCAG | 42484 | TUBB2A | 1 | CCAGCCGGGGC | 42862 |
| | | AGCC | | | | AGCC | |
| KCNG2 | 2 | GCCTGCTGTGGA | 42485 | TUBB2B | 1 | CCAGCCGGGGC | 42863 |
| | | TGA | | | | AGCC | |
| KCNG2 | 3 | GGGGAATCCCA | 42486 | TUBBP5 | 1 | CCAGCCGGGGC | 42864 |
| | | GAGCC | | | | AGCC | |
| KCNJ15 | 1 | TGTGCAGAATAT | 42487 | TULP1 | 1 | GAGGGCCAGGC | 42865 |
| | | GTC | | | | ACCCA | |
| KCNK10 | 1 | TCTTTCTTTCCTT | 42488 | UBXN10 | 1 | GCTGCTGCTGG | 42866 |
| | | GG | | | | TGA | |
| KCTD1 | 1 | GCCGGGGAGGA | 42489 | UCK2 | 1 | CTGAAGGGAGC | 42867 |
| | | GCTGG | | | | TC | |
| KCTD1 | 2 | GAACACCCGGCT | 42490 | UCK2 | 2 | ACCTCTCTTGC | 42868 |
| | | GGCCAC | | | | CCTTGT | |
| KCTD15 | 1 | GCCGGGAGGAG | 42491 | UNC13A | 1 | CAGAGCCGGCC | 42869 |
| | | CGG | | | | CGG | |
| KCTD15 | 2 | CCTCCCTTGGAT | 42492 | UNC45A | 1 | GGCGCCCTGGC | 42870 |
| | | CCTT | | | | GGC | |
| KIAA0319 | 1 | GCTGCTGCTGGT | 42493 | VANGL1 | 1 | CCACCTGGCTG | 42871 |
| | | GA | | | | GCA | |
| KIAA0895L | 1 | AGGCAAGGGAG | 42494 | VAV2 | 1 | GCCTGGAGGAG | 42872 |
| | | CTC | | | | CTGG | |
| KIAA1522 | 1 | CCACCTTCCGAC | 42495 | VAV3 | 1 | CCGGCCGGGGC | 42873 |
| | | CCC | | | | GCACG | |
| KIAA1549L | 1 | CAGGAGCCGGC | 42496 | VSTM4 | 1 | GGCCTGGGATT | 42874 |
| | | CCGGG | | | | CCTT | |
| KIF26A | 1 | GCAGCCGCCCTG | 42497 | VSTM4 | 2 | AGGCCTGGGAT | 42875 |
| | | CT | | | | TCCTT | |
| KIF5C | 1 | GCCCTGGAGGA | 42498 | VSTM4 | 3 | CGCCTCTCCAC | 42876 |
| | | GCTGG | | | | CAGCACC | |
| KLC3 | 1 | GCCCTGAGGAGC | 42499 | VWC2 | 1 | CCGGCCGGCCC | 42877 |
| | | TGG | | | | AGG | |
| KLF16 | 1 | GGCGCCCTGGTG | 42500 | VWC2 | 2 | ACCCCTCCGCG | 42878 |
| | | C | | | | GAG | |
| KLF16 | 2 | CTCCTCTCCACC | 42501 | VWF | 1 | GGCGCCCTGGC | 42879 |
| | | ACCCCC | | | | CAGC | |
| KLHL14 | 1 | TCCCTGTGGACC | 42502 | WASF2 | 1 | CCTTCTTTCCTG | 42880 |
| | | GAT | | | | GA | |
| KLK10 | 1 | GGCCCCCTGGTC | 42503 | WDR43 | 1 | TGCCTGGGATT | 42881 |
| | | TGT | | | | CCTG | |
| KRT86 | 1 | GTGGCAGGGCG | 42504 | WNT7A | 1 | CAGAGCCGGCC | 42882 |
| | | CCAC | | | | CGA | |
| KRTAP2-1 | 1 | CTCCTCTCCACA | 42505 | WWC1 | 1 | CCAGCCGGGGC | 42883 |
| | | GCCCA | | | | TCCC | |
| LAMA4 | 1 | CTGGCGGGGCTC | 42506 | WWC1 | 2 | GCCTGCTGCTG | 42884 |
| | | ACC | | | | AGGA | |
| LAMA5 | 1 | GTGGCAGGGCC | 42507 | WWOX | 1 | CGGGTCTCGTT | 42885 |
| | | ACGC | | | | TGGA | |
| LAMC2 | 1 | ATTCTGGCTGAT | 42508 | XCR1 | 1 | CCTTTCTTTCCT | 42886 |
| | | GTG | | | | AGT | |
| LCNL1 | 1 | GGAGGGCCAGG | 42509 | YEATS2 | 1 | CAGCCGGGGCA | 42887 |
| | | CCCCG | | | | GGT | |
| LDLR | 1 | TGGGATCCCAGG | 42510 | YPEL2 | 1 | AACCTGGCTGG | 42888 |
| | | CC | | | | TA | |
| LEFTY2 | 1 | CTGAGCCGGCCC | 42511 | ZC3H12B | 1 | CGAGTGCAGAG | 42889 |
| | | CGG | | | | CTATG | |
| LIMK1 | 1 | CAGAGCCGGCCC | 42512 | ZC3H12D | 1 | AGCGCCTGGTC | 42890 |
| | | AGC | | | | TGC | |
| LIMK1 | 2 | GCCCAGAGCCG | 42513 | ZDHHC8P1 | 1 | CTGGCCGGCCC | 42891 |
| | | GCCCAGC | | | | AGG | |
| LINC00319 | 1 | CACAGCCGGCCC | 42514 | ZFP14 | 1 | CAGAGCCGCCC | 42892 |
| | | AGC | | | | AGG | |
| LINC00528 | 1 | GCAGGCCGCCCT | 42515 | ZFP69 | 1 | GCGGCCGGGGC | 42893 |
| | | GCT | | | | TCACA | |
| LINC00540 | 1 | CGGGCCGGCCCA | 42516 | ZNF831 | 1 | CCCCTCAGAGA | 42894 |
| | | GG | | | | GGAAGAA | |

### Results of DUX4-Targeting Oligonucleotide and RNA Target Interaction

In some cases, a DUX4-targeting oligonucleotide or salt thereof comprising a modification when contacted with a DUX4 mRNA sequence may produce lower activity of a polypeptide encoded by the DUX4 mRNA sequence as compared to contacting an equivalent amount of an otherwise comparable DUX4-targeting oligonucleotide that lacks the modification with the DUX4 mRNA sequence. In some cases, the lower activity may be at least about 1.2-fold lower. In some cases, the lower activity may be at least about 1.5-fold lower. In some cases, the lower activity may be at least about 1.7-fold lower. In some cases, the lower activity may be at least about 2.0-fold lower. In some cases, the lower activity may be about: 1.2, 1.3, 1.4, 1.5, 1.6, 1.7, 1.8, 1.9, 2.0, 2.1, 2.2, 2.3, 2.4, 2.5-fold lower. In some cases, the lower activity may be from about 1.2-fold to about 2.0-fold lower. In some cases, the lower activity may be from about 1.1-fold to about 1.5-fold lower. In some cases, the lower activity may be from about 1.1-fold to about 2.5-fold lower. In some cases, the lower activity may be from about 1.2-fold to about 3.0-fold lower. In some cases, the lower activity may be at least about 1.2-fold to about at least 10-fold lower expression. In some cases, the lower activity may be at least about 14-fold lower. In some cases, the lower expression may be at least about 18-fold lower expression. In some cases, the lower activity may be about: 1.2, 2, 3, 4, 5, 6, 7, 8, 9, 10, 11, 12, 13, 14, 15, 16, 17, 18, 19, 20-fold lower. In some cases, the lower activity may be from about 1.2-fold to about 14-fold. In some cases, the lower activity may be from about 1.1-fold to about 20-fold lower. In some cases, the lower activity may be from about 1.2-fold to about 30-fold lower.

In some cases, the DUX4-targeting oligonucleotide or salt thereof, when contacted with the mRNA sequence, may produce at least about: 1.1, 1.2, 1.3, 1.4, 1.5, 1.6, 1.7, 1.8, 1.9, 2, 3, 4, 5, 6, 7, 8, 9, 10-fold lower expression of a polypeptide encoded by the mRNA sequence, as compared to contacting an equivalent amount of the otherwise comparable oligonucleotide with the mRNA sequence. Lower expression may be from about 1.2-fold to about 10-fold lower expression.

In some cases, the DUX4-targeting oligonucleotide or salt thereof, when contacted with the mRNA sequence, may produce at least about: 1.1, 1.2, 1.3, 1.4, 1.5, 1.6, 1.7, 1.8, 1.9, 2, 3, 4, 5, 6, 7, 8, 9,10-fold lower activity of a polypeptide encoded by the mRNA sequence, as compared to contacting an equivalent amount of the otherwise comparable oligonucleotide with the mRNA sequence. Lower activity may be from about 1.2-fold to about 10-fold lower activity.

In some cases, a DUX4-targeting oligonucleotide or salt thereof may comprise at least about a predicted thermal melting temperature of 45 to 65 degrees Celsius at physiological salt and pH. In some cases, a DUX4-targeting oligonucleotide or salt thereof may bind the RNA sequence at about 18, 19, 20, 21, 22, 23, 24, 25, 26, 27, 28, 29, 30, 31, 32, 33, 34, 35, 36, 37, 38, 39 or 40 degrees Celsius. In some cases, a DUX4-targeting oligonucleotide or salt thereof may bind the RNA sequence at a pH of about 6.6, 6.7, 6.8, 6.9, 7.0, 7.1, 7.2, 7.3, 7.4, 7.5, 7.6, 7.7, or 7.8.

### Subjects

In some aspects, a subject may comprise a mammal amenable to receive a composition as described herein comprising an engineered DUX4-targeting nucleic acid (such as in the form of an oligonucleotide) or treated by a method as described herein. Examples of such mammals may include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals may be any age or at any stage of development, for example a mammal may be neonatal, infant, adolescent, adult or in utero. Mammals may be male or female. In some cases, a human may be from about: 1 day to about 7 days old, 1 week to about 5 weeks old, 1 month to about 12 months old, 1 year to about 6 years old, 5 years to about 15 years old, 14 years to about 30 years old, 25 years to about 50 years old, 40 years to about 75 years old, 70 years to about 100 years old, 85 years old to about 110 years old or about 100 years to about 130 years old.

In some cases, a subject may not have been previously diagnosed with a disease or condition. In some cases, a subject may have been diagnosed with a disease or condition. In some cases, a subject may not have received a definitive diagnosis of a disease or condition. A subject may be at risk of developing a disease or condition (such as based at least in part on a genetic variant). A subject may have received a diagnostic test. A diagnostic test may include an imaging procedure, a blood count analysis, a tissue pathology analysis, a biomarker analysis, or any combination thereof.

The subject may be a patient, such as a patient being treated for a condition or a disease such as a neuromuscular disease. In certain cases, the subject may be predisposed to a risk of developing a condition or a disease such as neuromuscular disorder. The subject may be in remission from a condition or a disease, such as a neuromuscular disorder. The subject may be healthy.

In some aspects, a subject may be a subject in need thereof. In some aspects, a subject may have a disease such as treatment of facioscapulohumeral muscular dystrophy (FSHD) may include, for example, relieving the muscle weakness experienced by a mammal suffering from facioscapulohumeral muscular dystrophy (FSHD), and/or causing the regression or disappearance of muscle weakness.

### Administration and Treatment

In some aspects, DUX4-targeting oligonucleotides disclosed herein may be used to treat subjects such that the treatment results in: reduced malaise, an increase in energy, an increase in weight, a decrease in weight, an increase in muscle mass, an increase in, an increase in body flexibility, an increase in posture, an increase in range of movement, cessation of myotonia, abatement of muscle pain, or any combination thereof.

A subject in need thereof may be treated for a disease or condition. A treatment may be a pre-treatment, a prophylactic treatment, or a preventive treatment. Treatment may include administration to the subject in need thereof the DUX4-targeting oligonucleotide, a nucleic acid construct, a vector, or a pharmaceutical composition as described herein.

Treating may include administering an engineered DUX-4-targeted oligonucleotide highly conserved among patients and selected from SEQ. ID. NOs: 20,962-41,922 in the XML Sequence listing file submitted at the time of filing, and/or SEQ. ID. Nos: 41,923-42,115 as shown in Table 2, or any combination thereof.

Delivery may include direct application to the affected tissue or region of the body. Delivery may include a parenchymal injection, an intrathecal injection, an intraventricular injection, or an intracisternal injection. A composition provided herein may be administered by any method. A method of administration may be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery may include parenteral administration (including intravenous, subcutaneous, intrathecal, intraperitoneal, intramuscular, intravascular or infusion), oral administration, inhalation administration, intraduodenal administration, rectal administration. Delivery may include topical administration (such as a lotion, a cream, an ointment) to an external surface of a surface, such as a skin. In some instances, a subject may administer the composition in the absence of supervision. In some instances, a subject may administer the composition under the supervision of a medical professional (e.g., a physician, nurse, physician's assistant, orderly, hospice worker, etc.). In some cases, a medical professional may administer the pharmaceutical formulation. In some cases, the treatment of a neuromuscular disease such as facioscapulohumeral muscular dystrophy is by employing a composition which comprises a DUX4-targeting oligonucleotide, a vector comprising the oligonucleotide, or a pharmaceutical formulation as described below. Still further, a medicine may be prepared using a DUX4-targeting oligonucleotide, a vector comprising the oligonucleotide, or a pharmaceutical formulation as described below. The medicine may be used for the treatment or prevention of facioscapulohumeral muscular dystrophy.

Methods may of administration may include in vivo or in vitro delivery methods. Methods may include contacting a cell, such as a cell in vivo with the DUX4-targeting oligonucleotide, the nucleic acid construct, the vector, or the pharmaceutical composition as described herein. Methods may include contacting a cell, such as an isolated and purified cell (such as a cell in vitro) with the DUX4-targeting oligonucleotide, the nucleic acid construct, the vector, or the pharmaceutical composition as described herein. Methods may include contacting a tissue, such as an in vivo tissue or an isolated in vitro tissue, with the DUX4-targeting oligonucleotide, a nucleic acid construct, a vector, or a pharmaceutical composition as described herein.

Treatment may include more than one DUX4-targeting oligonucleotide delivered in a single dose. Delivery may be concurrent delivery, such as delivery more than one DUX4-targeting oligonucleotide in a single injection or in two separate injections at the same time. Delivery may be sequential, such as delivery of a first dose and a second dose that may be separated by a period of time, such as minutes, hours, days, weeks, or months.

Certain aspects of the disclosure pertain to administration of a DUX4-targeting oligonucleotide human cell may be a cell of head or neck tissue, a skin cell, a cervical cell, a prostate cell, a stem cell, a bone cell, a blood cell, a muscle cell, a fat cell, a nerve cell, an endothelial cell, sperm cell, egg cell, cancer cell, barrier cell, hormone-secreting cell, exocrine-secretory cell, epithelial cell, oral cell, sensory transducer cell, autonomic neuron cell, peripheral neuron cell, central nervous neuron cell, secretory cell, cardiac muscle cell, white blood cell, germ cell, nurse cell, kidney cell, or any combination thereof.

A tissue may be a sample that may be substantially healthy, substantially benign, or otherwise substantially free of a disease or a condition. A tissue may be a tissue removed from a subject, such as a tissue biopsy, a tissue resection, an aspirate (such as a fine needle aspirate), a tissue washing, a cytology specimen, a bodily fluid, or any combination thereof. A tissue may comprise cancerous cells, tumor cells, non-cancerous cells, or a combination thereof. A tissue may comprise a blood sample (such as a cell-free DNA sample). A tissue may be a sample that may be genetically modified.

Treatment may include treatment of a condition associated with a neuromuscular disease such as facioscapulohumeral muscular dystrophy. Treatment may result in reduced malaise, an increase in energy, an increase in weight, a decrease in weight, an increase in muscle mass, an increase in, an increase in body flexibility, an increase in posture, an increase in range of movement, cessation of myotonia, abatement of muscle pain, or any combination thereof.

Certain aspects of the disclosure pertain to delivery of an oligonucleotide such as a DUX4-targeting oligonucleotide with a vector. A vector may be employed to deliver the DUX4-targeting oligonucleotide, the nucleic acid construct, or any combination thereof. A vector may comprise DNA, such as double stranded DNA or single stranded DNA. A vector may comprise RNA. In some cases, the RNA may comprise a base modification. The vector may comprise a recombinant vector. The vector may be a vector that is modified from a naturally occurring vector. The vector may comprise at least a portion of a non-naturally occurring vector. In some cases, the vector may comprise a viral vector, a liposome, a nanoparticle, an exosome, an extracellular vesicle, or any combination thereof. In some cases, a viral vector may comprise an adenoviral vector, an adeno-associated viral vector (AAV), a lentiviral vector, a retroviral vector, a portion of any of these, or any combination thereof. In some cases, a nanoparticle vector may comprise a polymeric-based nanoparticle, an aminolipid based nanoparticle, a metallic nanoparticle (such as gold-based nanoparticle), a portion of any of these, or any combination thereof. In some cases, a vector may comprise an AAV vector. A vector may be modified to include a modified VP1 protein (such as an AAV vector modified to include a VP1 protein). An AAV may comprise a serotype—such as an AAV1 serotype, an AAV2 serotype, AAV3 serotype, an AAV4 serotype, AAV5 serotype, an AAV6 serotype, AAV7 serotype, an AAV8 serotype, an AAV9 serotype, a derivative of any of these, or any combination thereof.

In certain aspects, delivery of an oligonucleotide intended to function as an engineered DUX4-targeting oligonucleotide is through liposomal delivery. In certain instances, the liposome may be a positively charged liposome. In certain instances, the liposome may be a negatively charged liposome. In other instances, the delivery of engineered DUX4-targeting oligonucleotide is a polymer delivery. In other instances, the engineered DUX4-targeting oligonucleotide delivery is a dendrimer mediated delivery. In other instances, the delivery of an engineered DUX4-targeting oligonucleotide is via microinjection, electroporation, ultrasound, gene gun or hydrodynamic applications. In other instances, the delivery of an engineered DUX4-targeting oligonucleotide is via conjugation to or association with a nanoparticle.

### Pharmaceutical Formulations

In some aspects, a wide variety of pharmaceutical formulations to deliver an engineered DUX4-targeting oligonucleotide target may be employed.

A pharmaceutical formulation may comprise a pharmaceutically acceptable excipient, diluent, carrier, or a combination thereof.

A carrier of a pharmaceutical formulation may be, in certain cases, a solid carrier and may comprise lactose, terra alba, sucrose, talc, gelatin, agar, pectin, acacia, magnesium stearate, stearic acid and the like. In other cases, the carrier is a liquid carrier and may comprise phosphate buffered saline solution, syrup, oil, peanut oil, olive oil, water, emulsions, a wetting agent, a sterile solution, or any combination thereof.

In some aspects regarding pharmaceutical formulations, a pharmaceutical formulation may comprise a pharmaceutically acceptable diluent. A diluent may comprise for example, sterile distilled water, deionized water, physiological saline, Ringer's solutions, dextrose solution, a cell growth medium, phosphate buffered saline (PBS), or any combination thereof.

In some aspects regarding pharmaceutical formulations, a pharmaceutical formulation may comprise a excipient. In instances concerning the excipient, the excipient may comprise a pH agent, a stabilizing agent, a buffering agent, a solubilizing agent, or any combination thereof. An excipient may comprise a surfactant, a sugar, an amino acid, an antioxidant, a salt, a non-ionic surfactant, a solubilizer, a triglyceride, an alcohol, or any combination thereof. An excipient may comprise sodium carbonate, acetate, citrate, phosphate, polyethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. An excipient may be an excipient described in the Handbook of Pharmaceutical Excipients, American Pharmaceutical Association (1986).

Included in the present disclosure may be salts, including pharmaceutically acceptable salts, of the compositions described herein. The compounds or compositions of the present disclosure that may possess a sufficiently acidic, a sufficiently basic, or both functional groups, may react with any of a number of in-organic bases, inorganic acids, or organic acids, to form a salt. Alternatively, compositions containing compounds that are inherently charged, such as those with quaternary nitrogen, may form a salt with an appropriate counterion, e.g., a halide such as bromide, chloride, or fluoride, particularly bromide.

A pharmaceutical composition may comprise a first active ingredient. The first active ingredient may comprise a DUX4-targeting oligonucleotide as described herein. The pharmaceutical composition may be formulated in unit dose form. The pharmaceutical composition may comprise a pharmaceutically acceptable excipient, diluent, or carrier. The pharmaceutical composition may comprise a second, third, or fourth active ingredient, such as a second DUX4-targeting oligonucleotide.

In some cases, an engineered DUX4-targeting oligonucleotide or salt thereof comprising a modification when stored in a closed container placed in a room for a time period will remain at least about 80% of an initial amount of the engineered DUX4-targeting oligonucleotide or salt thereof. In some cases, the engineered DUX4-targeting oligonucleotide will remain at least about 70% the initial amount. In some cases, the engineered DUX4-targeting oligonucleotide will remain at least about 90% the initial amount. In some cases, the engineered DUX4-targeting oligonucleotide will remain at least about: 60%, 65%, 70%, 75%, 80%, 85%, 90%, 95%, 99%. In some cases, the engineered DUX4-targeting oligonucleotide may be at least about 60% to about at least 80%. In some cases, engineered DUX4-targeting oligonucleotide may be at least about 80% to at least about 99%. In some cases, the time period of storage may be at least 1 month. In some cases, the time period of storage may be at least about 3 months. In some cases, the time period of storage may be at least about 1 year. In some cases, the time period of storage may be at least about 1, 2, 4, 6, 8, 12, 18, 24, 36, 48 or 60 months. In some cases, the time period of storage may be at least about 1 month to about at least 1 year. In some cases, the time period of storage may be at least about 6 months to at least about 2 years. In some cases, the time period of storage may be at least about 1 month to at least about 5 years.

In some aspects, a pharmaceutical composition may be administered to a subject at a suitable unit dose. The pharmaceutical composition may be in unit dose form. In some cases, unit dose may be meant to refer to pharmaceutical drug products in the form in which they are marketed for use, with a specific mixture of active ingredients and inactive components, diluents, or excipients, in a particular configuration, and apportioned into a particular dose to be delivered. In some instances, unit dose may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. In some instances, the term unit dose may also sometimes refer to the particles comprising a pharmaceutical composition, and to any mixtures involved. In some cases, types of unit doses may vary with the route of administration for drug delivery, and the substance(s) being delivered. In some aspects, administration may comprise intravenous, intraperitoneal, intra-arterial, intertumoral, subcutaneous, intramuscular, intranasal, topical, oral, or intradermal administration. In some cases, administration may comprise inhalation administration. In some aspects, a dosage regimen may be determined by an attending physician and clinical factors. In some aspects, a dosage for a subject may depend upon many factors, including a subject's size, body surface area, age, sex, general health, a compound to be administered, a time and route of administration, other drugs being administered concurrently, or any combination thereof. In some aspects, a range of a dose may comprise 0.001 to 1000 μg. In some aspects, a dose may be below or above such a range. In some aspects, a regimen as a regular administration of a pharmaceutical composition may be in a range of 1 μg to 10 mg. In some aspects, a regimen as a regular administration of a pharmaceutical composition may be in a range of 10² units to 10¹² units per day, week or month. In some cases, a unit may be a vector or an ASO. In some aspects, if a regimen comprises a continuous infusion, it may also be in a range of 1 μg to 10,000 mg of pharmaceutical composition or engineered polynucleotide or DNA encoding the engineered polynucleotide or vector containing or encoding the engineered polynucleotide per kilogram of body weight per minute, respectively. In certain instances, the range is from 1 mg per kilogram of body weight to 1000 mg per kilogram of body weight. In some aspects, progress may be monitored by periodic assessment.

In some aspects of the disclosure, when a pharmaceutical composition is a liquid it may be administered in a liquid dose form such as about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml, or about 750 ml to 1000 ml.

In some aspects, a composition described herein may be administered one or more days to a subject in need thereof. In some aspects, administration may occur for about: 1, 2, 3, 4,5,6,7,8,9, 10, 11, 12, 13, 14, 15, 16, 17, 18, 19, 20, 21, 22, 23, 24, 25, 26, 27, 28, 29, 30 or about 31 days. In some aspects, administration may occur for about: 1, 2, 3, 4, 5, 6, 7, 8, 9, 10, 11, 12, 13, 14, 15, 16, 17, 18, 19, 20, 21, 22, 23, or about 24 months. In some aspects, administration may occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 or more years. In some cases, administration may occur for life. In some aspects, a pharmaceutical composition described herein may be administered on 2, 3, 4, 5, 6, 7, 8, 9, 10, 11, 12, 13, 14, 15, 16, 17, 18, 19 or 20 or more days. In some cases, a composition described herein may be administered on consecutive days or on nonconsecutive days. In some cases, a composition described herein may be administered to a subject more than one time per day. In some instances, a composition described herein may be administered to a subject: 1, 2, 3, 4, 5, 6, 7, 8, 9, or 10 or more times per day.

In some aspects, disclosed herein are methods of use for compositions as disclosed herein. In some aspects, a daily oral dosage regimen may be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen may comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen may be from about 0.1 mg to about 500 mg. In some aspects, a daily dosage regimen may be from about 0.01 mg/kg to about 1,000 mg/kg per day. In some aspects, an optimal quantity and spacing of individual dosages of a composition may be determined by a nature and extent of a condition being treated, a form, route and site of administration, and a particular subject being treated, and that such optimums may preferably be determined by a method described herein. In some aspects, a number of doses of compositions given per day for a defined number of days, may be ascertained by those skilled in the art using conventional course of treatment determination tests. In some aspects, a dosage regimen may be determined by an attending physician and other clinical factors. In some aspects, dosages for any one subject may depend upon many factors. In some aspects, factors affecting dosage may comprise a subject's size, body surface area, age, a particular compound to be administered, sex, time and route of administration, general health, other drugs being administered concurrently or any combination thereof. In some aspects, progress may be monitored by periodic assessment.

A pharmaceutical formulation may be administered a daily oral dosage regimen may be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen may comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen may be from about 0.1 mg to about 500 mg. In some aspects, a daily dosage regimen may be from about 0.01 mg/kg to about 1,000 mg/kg per day. In some aspects, an optimal quantity and spacing of individual dosages of a composition may be determined by a nature and extent of a condition being treated, a form, route and site of administration, and a particular subject being treated, and that such optimums may preferably be determined by a method described herein. In some aspects, a number of doses of compositions given per day for a defined number of days, may be ascertained by those skilled in the art using conventional course of treatment determination tests. In some aspects, a dosage regimen may be determined by an attending physician and other clinical factors. In some aspects, dosages for any one subject may depend upon many factors. In some aspects, factors affecting dosage may comprise a subject's size, body surface area, age, a particular compound to be administered, sex, time and route of administration, general health, other drugs being administered concurrently or any combination thereof. In some aspects, progress may be monitored by periodic assessment.

A composition or formulation may be used herein for treating or preventing a neuromuscular disease comprising an engineered DUX4-targeting oligonucleotide configured to hybridize to an RNA comprising a portion of a RNA transcript, wherein the engineered DUX4-targeting oligonucleotide comprises at least 70% sequence identity to an oligonucleotide of any one of SEQ. ID. NOs: 41,923-42,115, a vector encoding or comprising said oligonucleotide, and a pharmaceutically acceptable: excipient, diluent, or carrier. In certain cases, the neuromuscular disease is facioscapulohumeral muscular dystrophy. In other aspects, may call for the use of an engineered DUX4-targeting oligonucleotide configured to hybridize to an RNA comprising a portion of a RNA transcript, wherein the engineered DUX4-targeting oligonucleotide comprises at least 70% sequence identity to an oligonucleotide of any one of SEQ. ID. NOs: 41,923-42,115, and a pharmaceutically acceptable: excipient, diluent, or carrier in the preparation of a medicament for the treatment and prevention of facioscapulohumeral muscular dystrophy.

### Co Therapies

In some aspects, disclosed herein are methods of administering a DUX4-targeting oligonucleotide or salt thereof to a subject in combination with a co-therapy. In some aspects, one or more additional co-therapies may be administered concurrently. In some aspects, one or more additional therapeutics may be administered consecutively. In some cases, an co-therapy may comprise immunotherapy, hormone therapy, cryotherapy, surgical procedure or any combination thereof. A co-therapy may include administration of a pharmaceutical composition, such as a small molecule. A co-therapy may include administration of a pharmaceutical composition, such as one or more antibiotics. A co-therapy may comprise administration of a muscle relaxant, an anti-depressant, a steroid, an opioid, a cannabis-based therapeutic, acetaminophen, a non-steroidal anti-inflammatory, a neuropathic agent, a cannabis, a progestin, a progesterone, or any combination thereof. A neuropathic agent may comprise gabapentin. A non-steroidal anti-inflammatory may comprise naproxen, ibuprofen, a COX-2 inhibitor, or any combination thereof. A second therapy may comprise administration of a biologic agent, cellular therapy, regenerative medicine therapy, a tissue engineering approach, a stem cell transplantation or any combination thereof. A co-therapy may comprise a medical procedure. A medical procedure may comprise an epidural injection (such as a steroid injection), acupuncture, exercise, physical therapy, an ultrasound, a surgical therapy, a chiropractic manipulation, an osteopathic manipulation, a chemonucleolysis, or any combination thereof. A co-therapy may comprise use of a breathing assist device or a ventilator. A co-therapy may comprise administration of a regenerative therapy or an immunotherapy such as a protein, a stem cell, a cord blood cell, an umbilical cord tissue, a tissue, or any combination thereof. A second therapy may comprise an anti-inflammatory compound, or an anti-fibrosis compound such as pirfenidone, nintedanib, tocilizumab, mycophenolate mofetil/mycophenolic acid prednisone, azathioprine, or a combination thereof. A second therapy may comprise a biosimilar.

In some aspects, when a co-therapy is a pharmaceutical agent, the pharmaceutical agent included in a pharmaceutical composition in the form of a fixed dose combination drug.

In some cases, a co-therapeutic dose regimen may be administered for a duration of about 1 week, about 2 weeks, about 3 weeks, about 4 weeks, about 6 weeks, about 7 weeks, about 8 weeks, about 9 weeks, about 10 weeks, or about 12 weeks. In some cases, a dose regimen may be administered for a duration of about 1 month, about 2 months, about 3 months, about 4 months, about 6 months, about 7 months, about 8 months, about 9 months, about 10 months, or about 12 months. In some cases, a dose regimen may be administered for a duration of about 1 year, about 2 years or more than about 3 years.

In some aspects, disclosed herein are methods of use for co-therapy compositions as disclosed herein. In some aspects, a daily oral dosage regimen may be from about 0.1 milligram per kilogram (mg/kg) to about 80 mg/kg of total body weight, from about 0.2 mg/kg to about 30 mg/kg, or from about 0.5 mg/kg to about 15 mg/kg. In some aspects, a daily parenteral dosage regimen may comprise from about 0.1 mg/kg to about 10,000 mg/kg of total body weight, from about 0.2 mg/kg to about 5,000 mg/kg, or from about 0.5 mg/kg to about 1,000 mg/kg. In some aspects, a daily topical dosage regimen may be from about 0.1 mg to about 500 mg. In some aspects, a daily dosage regimen may be from about 0.01 mg/kg to about 1,000 mg/kg per day. In some aspects, an optimal quantity and spacing of individual dosages of a composition may be determined by a nature and extent of a condition being treated, a form, route and site of administration, and a particular subject being treated, and that such optimums may preferably be determined by a method described herein. In some aspects, a number of doses of compositions given per day for a defined number of days, may be ascertained by those skilled in the art using conventional course of treatment determination tests. In some aspects, a dosage regimen may be determined by an attending physician and other clinical factors. In some aspects, dosages for any one subject may depend upon many factors. In some aspects, factors affecting dosage may comprise a subject's size, body surface area, age, a particular compound to be administered, sex, time and route of administration, general health, other drugs being administered concurrently or any combination thereof. In some aspects, progress may be monitored by periodic assessment.

In some aspects, a co-therapy described herein may be administered one or more days to a subject in need thereof. In some aspects, administration may occur for about: 1, 2, 3, 4,5,6,7,8,9, 10, 11, 12, 13, 14, 15, 16, 17, 18, 19, 20, 21, 22, 23, 24, 25, 26, 27, 28, 29, 30 or about 31 days. In some aspects, administration may occur for about: 1, 2, 3, 4, 5, 6, 7, 8, 9, 10, 11, 12, 13, 14, 15, 16, 17, 18, 19, 20, 21, 22, 23, or about 24 months. In some aspects, administration may occur for about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 or more years. In some cases, administration may occur for life. In some aspects, a pharmaceutical composition described herein may be administered on 2, 3, 4, 5, 6, 7, 8, 9, 10, 11, 12, 13, 14, 15, 16, 17, 18, 19 or 20 or more days. In some cases, a composition described herein may be administered on consecutive days or on nonconsecutive days. In some cases, a composition described herein may be administered to a subject more than one time per day. In some instances, a composition described herein may be administered to a subject: 1, 2, 3, 4, 5, 6, 7, 8, 9, or 10 or more times per day.

In some aspects, a regimen as a regular administration of a pharmaceutical agent may be in a range of 1 μg to 10 mg. In some aspects, a regimen as a regular administration of a pharmaceutical composition may be in a range of 10² units to 10¹⁰ units per day, week or month. In some aspects, if a regimen comprises a continuous infusion, it may also be in a range of 1 μg to 10,000 mg of pharmaceutical agent. In certain instances, the range is from 1 mg per kilogram of body weight to 1000 mg per kilogram of body weight. In some aspects, progress may be monitored by periodic assessment.

### Kits

A kit may include the DUX4-targeting oligonucleotide in a container, the nucleic acid construct in a container, the vector in a container, the pharmaceutical composition in a container. A kit may include more than one DUX4-targeting oligonucleotide in a container, more than one vector in a container, more than one nucleic acid construct in a container, or more than one pharmaceutical composition in a container. In some cases, a container may be a plastic, a glass, or a metal container. A container may comprise a syringe, a vial, an ampule, a bag, ajar, and the like.

A kit may include a plurality of containers, each container comprising one or more DUX4-targeting oligonucleotides, or nucleic acid constructs, or vectors, or pharmaceutical compositions. A kit may include an excipient or a diluent or a buffer or a liquid or gel-like medium for storage of the DUX4-targeting oligonucleotide, the nucleic acid construct, the vector, or the pharmaceutical composition. A kit may include an excipient or a diluent or a buffer or a liquid or gel-like medium for in vivo delivery to a subject of the DUX4-targeting oligonucleotide, the nucleic acid construct, the vector, or the pharmaceutical composition. An excipient or diluent or buffer or liquid or gel-like medium may be included in the container housing the DUX4-targeting oligonucleotide (or nucleic acid construct or vector or pharmaceutical composition) or housed in a separate container. A kit may include a delivery vehicle, such as a syringe or needle. A kit may include one or more reagents for a downstream analysis.

In some cases, at least about: 70%, 75%, 80%, 85%, 90%, 95% of an initial amount of the DUX4-targeting oligonucleotide or salt thereof remains when the DUX4-targeting oligonucleotide or salt thereof may be stored in a closed container placed in a room for a time period of at least about: 1 month, 2 months, 3 months, 4 months, 5 months, 6 months at about from about 21 to about 25 degrees Celsius (such as about: 21, 22, 23, 24, 25 degrees Celsius) with a relative atmospheric humidity of from about 45% to about 55% (such as about: 45%, 46%, 47%, 48%, 49%, 50%, 51%, 52%, 53%, 54%, 55%). In some cases, the time period may be from about 1 month to about 1 year. In some cases, the time period may be from about 1 month to about 2 year. In some cases, the time period may be from about 1 month to about 6 months. In some cases, the time period may be from about 1 month to about 3 year. In some cases, the time period may be from about 1 month to about 9 months.

### Diagnostics

In some cases, a method may further comprise diagnosing a subject as having the disease. In some cases, a diagnosing may comprise employing an in vitro diagnostic. In some cases, the in vitro diagnostic may be a companion diagnostic. In other instances, the diagnosing may comprise an in vivo diagnostic.

A diagnostic test may comprise an imaging procedure, a blood count analysis, a tissue pathology analysis, a biomarker analysis, a biopsy, a magnetic resonance image procedure, a physical examination, a urine test, an ultrasonography procedure, a genetic test, a liver function test, a positron emission tomography procedure, a X-ray, serology, an angiography procedure, an electrocardiography procedure, an endoscopy, a diagnostic polymerase chain reaction test (PCR), a pap smear, a hematocrit test, a skin allergy test, a urine test, a colonoscopy, an enzyme-linked immunosorbent assay (ELISA), microscopy analysis, bone marrow examination, rapid diagnostic test, pregnancy test, organ function test, toxicology test, infectious disease test, bodily fluids test, or any combination thereof.

### Computer Control Systems

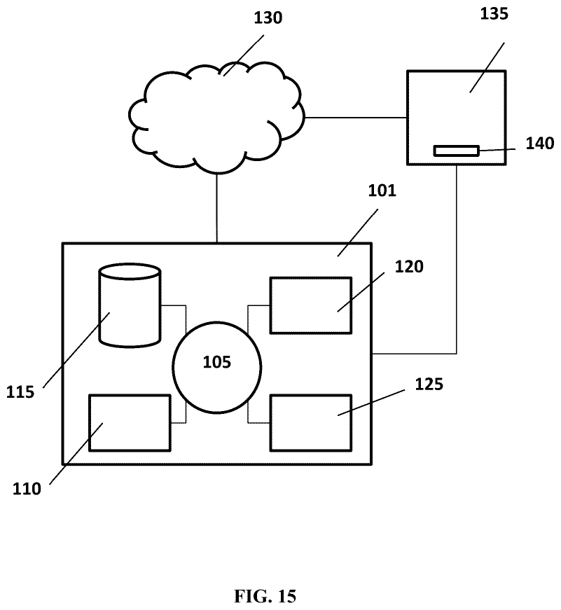

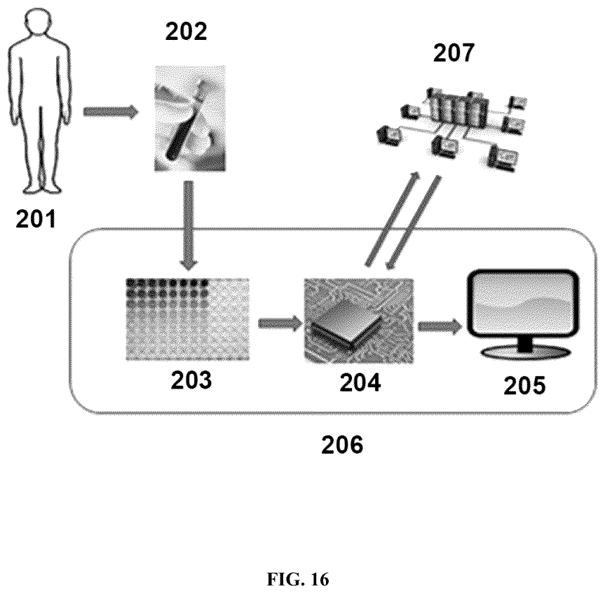

The present disclosure provides computer control systems that are programmed to implement methods of the disclosure. FIG. 15 shows a computer system 101 that is programmed or otherwise configured to predict or confirm efficacy of various constructs for therapeutic effect, such as in the treatment of FSHD. The computer system 101 may regulate various aspects of the present disclosure, such as, for example, modeling or identifying constructs for various therapeutic targets, modeling efficacy or stability of constructs, or any combination thereof. The computer system 101 may be an electronic device of a user or a computer system that is remotely located with respect to the electronic device. The electronic device may be a mobile electronic device.

The computer system 101 includes a central processing unit (CPU, also “processor” and “computer processor” herein) 105, which may be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 may be a data storage unit (or data repository) for storing data. The computer system 101 may be operatively coupled to a computer network (“network”) 130 with the aid of the communication interface 120. The network 130 may be the Internet, an internet and/or extranet, or an intranet and/or extranet that is in communication with the Internet. The network 130 in some cases is a telecommunication and/or data network. The network 130 may include one or more computer servers, which may enable distributed computing, such as cloud computing. The network 130, in some cases with the aid of the computer system 101, may implement a peer-to-peer network, which may enable devices coupled to the computer system 101 to behave as a client or a server.

The CPU 105 may execute a sequence of machine-readable instructions, which may be embodied in a program or software. The instructions may be stored in a memory location, such as the memory 110. The instructions may be directed to the CPU 105, which may subsequently program or otherwise configure the CPU 105 to implement methods of the present disclosure. Examples of operations performed by the CPU 105 may include fetch, decode, execute, and writeback.

The CPU 105 may be part of a circuit, such as an integrated circuit. One or more other components of the system 101 may be included in the circuit. In some cases, the circuit is an application specific integrated circuit (ASIC).

The storage unit 115 may store files, such as drivers, libraries and saved programs. The storage unit 115 may store user data, e.g., user preferences and user programs. The computer system 101 in some cases may include one or more additional data storage units that are external to the computer system 101, such as located on a remote server that is in communication with the computer system 101 through an intranet or the Internet.

The computer system 101 may communicate with one or more remote computer systems through the network 130. For instance, the computer system 101 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 101 via the network 130.

Methods as described herein may be implemented by way of machine (e.g., computer processor) executable code stored on an electronic storage location of the computer system 101, such as, for example, on the memory 110 or electronic storage unit 115. The machine executable or machine-readable code may be provided in the form of software. During use, the code may be executed by the processor 105. In some cases, the code may be retrieved from the storage unit 1115 and stored on the memory 110 for ready access by the processor 105. In some situations, the electronic storage unit 115 may be precluded, and machine-executable instructions are stored on memory 110.

The code may be pre-compiled and configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 may include or be in communication with an electronic display 135 that comprises a user interface (UI) 140 for providing, for example, one or more results (immediate results or archived results from a previous method), one or more user inputs, a reference value or derivative thereof from a library or database, or any combination thereof. Examples of UI's include, without limitation, a graphical user interface (GUI) and web-based user interface.

In some cases, as shown in FIG. 15, a sample 202 containing a genetic material may be obtained from a subject 201, such as a human subject. A sample 202 may be subjected to one or more methods as described herein, such as performing an assay. In some cases, an assay may comprise sequencing (such as nanopore sequencing), genotyping, hybridization, amplification, labeling, or any combination thereof. One or more results from a method may be input into a processor 204. One or more input parameters such as a sample identification, subject identification, sample type, a reference, or other information may be input into a processor 204. One or more metrics from an assay may be input into a processor 204 such that the processor may produce a result, such as a diagnosis of neuromuscular disease or a recommendation for a treatment. A processor may send a result, an input parameter, a metric, a reference, or any combination thereof to a display 205, such as a visual display or graphical user interface. A processor 204 may (i) send a result, an input parameter, a metric, or any combination thereof to a server 207, (ii) receive a result, an input parameter, a metric, or any combination thereof from a server 207, (iii) or a combination thereof.

Methods and systems of the present disclosure may be implemented by way of one or more algorithms. An algorithm may be implemented by way of software upon execution by the central processing unit 105. The algorithm can, for example, determine optimized constructs via supervised learning to optimize therapeutic efficacy, stability, or other attribute of one or more constructs.

### EXAMPLES

### Example 1: DUX4 Sequence from Skeletal Muscle Samples

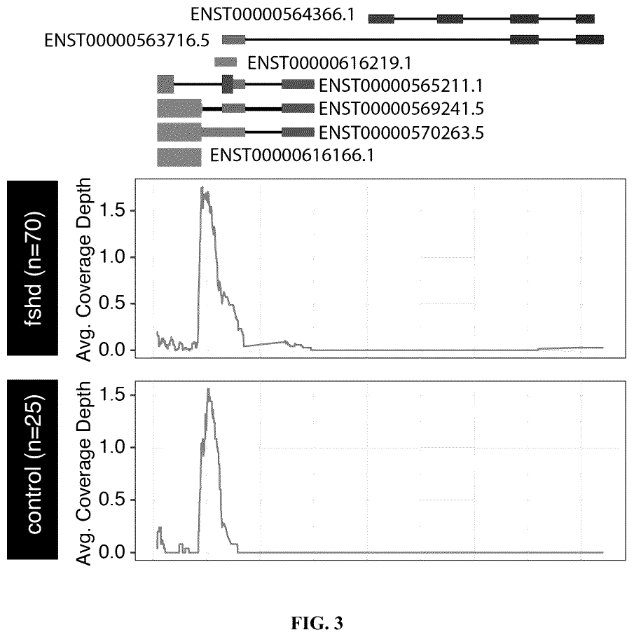

An analysis was performed from RNA-seq data of a total of 95 skeletal muscle samples of which 70 were derived from FSHD patients and 25 from healthy individuals. The samples used were from the following three publicly available datasets: Yao et al. 2014 (28) Wong et al. 2020 (17) and Wang et al. 2019 (29). The results of this analysis are shown in FIG. 3. However, this approach was not successful in generating enough data to predict RNA sequence variation in patients from most for the DUX4 coding sequence due to the fact that DUX4 is expressed at such a low level that only 1 or 2 reads was identified per sequence sample. To confidently predict sequence variation between individuals 50 to 100× read counts are normally required per samples. Only a small region of DUX4 located in exon 1 met this criteria.

### Example 2: DUX4 Sequence from Testis Samples

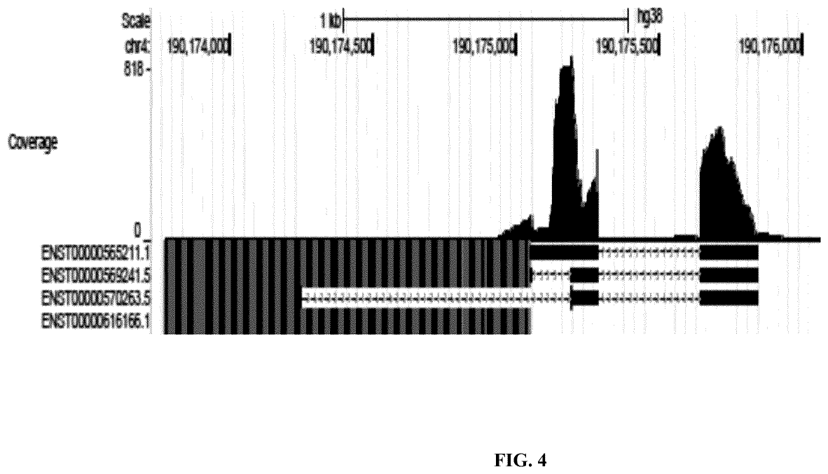

We decided to test this negative hypothesis and analyzed RNA-seq data of testis samples from 206 individuals (30). Unexpectedly, this dataset was sufficient to predict variance across exons 1,2,3 of the muscle specific transcripts with mean coverage of 117× across this sequence (FIG. 4). From this dataset we were able to several regions of the DUX4 coding sequence that are >85% conserved displaying promise in the approach. However, several regions of DUX4 still did not have enough coverage to accurately predict conservation, likely due to the difference in splice isoforms.

### Example 3: DUX4 Sequence from Combined Muscle and Testis Samples Database

To solve this problem, we took a very unique and unprecedented approach and combined the muscle RNA-seq and the testis RNA-seq into one merged dataset and performed the analysis. For this final analysis we were able to obtain 486 testis samples from GTEX and utilized the 95 skeletal muscle samples from Example 1. This final analysis generated the best data yielding read coverage of >50× for over 97% of the DUX4 gene allowing accurate prediction of DUX4 target site and OTN pairs that are greater than 85% conserved among patients (Table 4). As described above all resulting OTN sequences and pared DUX4 target site sequences, all represented in DNA form, are submitted as an xml file encompassing SEQ. ID. NOs 1-41,922. This data will be useful in identifying suitable stretches of 15-25 bases in the DUX4 sequence that are conserved among the majority for FSHD patient and selection for OTN drug development.

TABLE 4
Conserved DUX4 Regions

190173724-190173916, 190173917-190173950, 190173951-190174011, 190174012-
190174073, 190174074-190174231, 190174232-190174291, 190174292-190174343,
190174344-190174517, 190174518-190174941, 190174942-190175068, 190175069-
190175149, 190175150-190175197, 190175198-190175218, 190175219-190175285,
190175286-190175414, 190175415-190175588, 190175589-190175654, 190175655-
190175713, 190175714-190175816, 190175817-190175918, 190175919-190176359,
190176360-190176589, 190176590-190178325, 190178326-190178341, 190178342-
190178384, 190178385-190178402, 190178403-190178458, 190178459-190178528,
190178529-190178585, 190178586-190178886, 190178887-190179164, 190179165-
190179573, 190179574-190179602, 190179603-190179746, 190179747-190179926,
190179927-190180010, 190180011-190180125, 190180126-190180225, 190180226-
190181024, 190181025-190181092, 190181093-190181112, 190181113-190181219,
190181220-190181254, 190181255-190181291, 190181292-190181441, 190181442-
190181625, 190181626-190181656, 190181657-190181909, 190181910-190181954,
190181955-190182178, 190182179-190182208, 190182209-190182254, 190182255-
190182357, 190182358-190182437, 190182438-190183539, 190183540-190183738,
190183739-190183811, 190183812-190183834, 190183835-190184392, 190184393-
190184588, 190184589-190184611, 190184612-190184807, 190184808-190184858,
190184859-190185155, 190185156-190185731, 190185732-190185762, 190185763-
190185826, 190185827-190185947.

Regarding Table 4, contiguous sequence encoding DUX4 on chromosome 4q35 that are >85% conserved among individuals and could serve as target sites for ONTs targeting DUX4 for the treatment of FSHD. The DNA sequence for DUX4 and listed coordinates align with Ensemble release 101 (GRCh38.p13).

Referring to FIG. 1, this figure depicts genetic modifications leading to FSHD. In FSHD type 1 is the result of a deletion of the D4Z4 repeats on chromosome 4q35 from around 100 to less than 11 repeats leading to opening of chromatin and expression of DUX4. FSHD Type 2 is the result of a loss of function mutation in the epigenetic factor SMCHD1 leading to demethylation D4Z4 repeats on 4q35, opening of chromatin and expression of DUX4.

Referring to FIG. 2, this figure shows alternately spliced DUX4 transcripts originating from D4Z4 regions. ENST00000616166.1, ENST00000569241.5, and ENST00000570263.5 are associated with FSHD when expressed in muscle tissue. Transcripts ENST00000565211.1, ENST00000563716.5,and ENST00000564366.1 are normally expressed in other tissues and are not associated with the disorder. For example, ENST00000563716.5 is expressed in the testes.

Referring to FIG. 3, this figure shows read coverage from RNA-Seq data of alternately spliced DUX4 transcripts from FSHD and Healthy muscle biopsy tissue.

Referring to FIG. 4, this figure shows read coverage from RNA-Seq data of alternately spliced DUX4 transcripts from the Testis. Alternately spliced DUX4 transcripts ENST00000616166.1, ENST00000569241.5, and ENST00000570263.5 are associated with FSHD when expressed in muscle tissue.

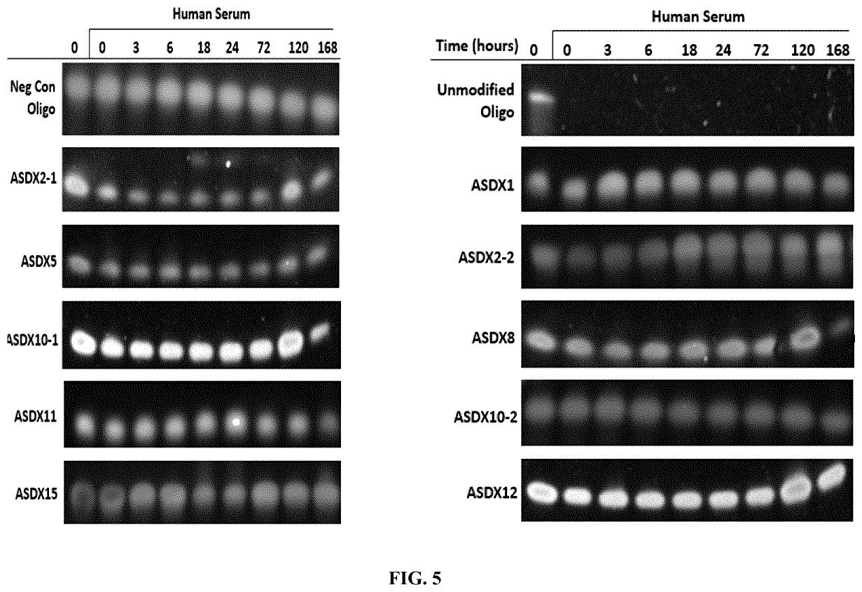

Referring to FIG. 5, this figure shows that chemical modifications DUX4 targeted ASOs may improve ASO stability to biological nucleases. DUX4 targeted ASOs were incubated in 10% human serum at 37° C. for the indicated lengths of time. ASO stability at each time point was visualized by denaturing Urea-PAGE. Unmodified Oligo, shows an example of an unmodified DNA nucleic acid that has very low half-life, Neg Con Oligo shows an ASO that does not target DUX4 but has similar chemical modifications while the other panels show chemically engineered ASOs that target DUX4. An exceptional example displays stability to biological nucleases out to 7 days (168 hours).

Referring to Table 5, this table shows the calculated half-life of chemically modified ASOs targeting DUX4 to biological nucleases. DUX4 ASOs were incubated in 10% human serum at 37° C. at different time points and visualized via Urea-PAGE. Densitometry was performed on each time point and ASO stability at each time point was calculated and averaged based on the formula N_{(T)}=N₀(½)_{t/t(1/2)} where N_{(T)} is signal at time point t. No is the signal at the start before incubation with nucleases and t (½) is the half-life. Unmodified refers to non-chemically modified RNA while Neg Con Oligo refers to an ASO that is chemically modified but does not target DUX4.

TABLE 5
Human Serum Half Life

| | Half Life | | Half Life |
|---|---|---|---|
| ASO ID | (Hours) | ASO ID | (Hours) |
| Unmodified | 0.05 | AS-DX-059-1 | >720 |
| Neg Con Oligo | >720 | AS-DX-098-1 | 427 |
| AS-DX-001 | 119 | AS-DX-099-1 | >720 |
| AS-DX-002-1 | 15 | AS-DX-027-2 | >720 |
| AS-DX-002-2 | 291 | AS-DX-027-3 | >720 |
| AS-DX-005-1 | 63 | AS-DX-027-4 | >720 |
| AS-DX-008-1 | 124 | AS-DX-100-01 | >720 |
| AS-DX-010-1 | 150 | AS-DX-101-01 | >720 |
| AS-DX-010-2 | 505 | AS-DX-102-01 | >720 |
| AS-DX-011-1 | 282 | AS-DX-103-01 | >720 |
| AS-DX-012-1 | 700 | AS-DX-105-01 | 353 |
| AS-DX-015-1 | 336 | AS-DX-033-2 | 601 |
| AS-DX-101-1 | 139 | AS-DX-033-3 | >720 |
| AS-DX-102-1 | 238 | AS-DX-107-1 | 433 |
| AS-DX-103-1 | 77 | AS-DX-108-1 | 334 |
| AS-DX-104-1 | 108 | AS-DX-50-3 | >720 |
| AS-DX-105-1 | 45 | AS-DX-50-4 | >720 |
| AS-DX-106-1 | 256 | AS-DX-110-1 | 668 |
| AS-DX-107-1 | 128 | AS-DX-111-1 | 540 |
| AS-DX-108-1 | 118 | AS-DX-59-2 | 611 |
| AS-DX-120-1 | 111 | AS-DX-112-1 | >720 |
| AS-DX-007-1 | >720 | AS-DX-59-3 | >720 |
| AS-DX-016-1 | >720 | | |
| AS-DX-018-1 | 387 | | |
| AS-DX-023-1 | 339 | | |
| AS-DX-025-1 | 857 | | |
| AS-DX-027-1 | 487 | | |
| AS-DX-028-1 | 221 | | |
| AS-DX-029-1 | 377 | | |
| AS-DX-030-1 | 538 | | |
| AS-DX-033-1 | 198 | | |
| AS-DX-038-1 | 174 | | |
| AS-DX-040-1 | 322 | | |
| AS-DX-041-1 | >720 | | |
| AS-DX-050-1 | 350 | | |
| AS-DX-052-1 | 91 | | |
| AS-DX-057-1 | 401 | | |

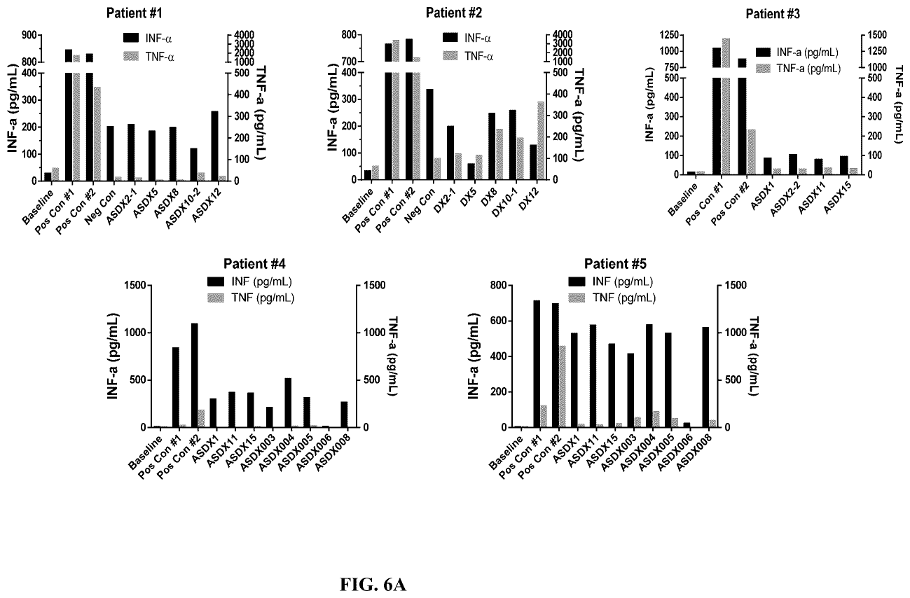

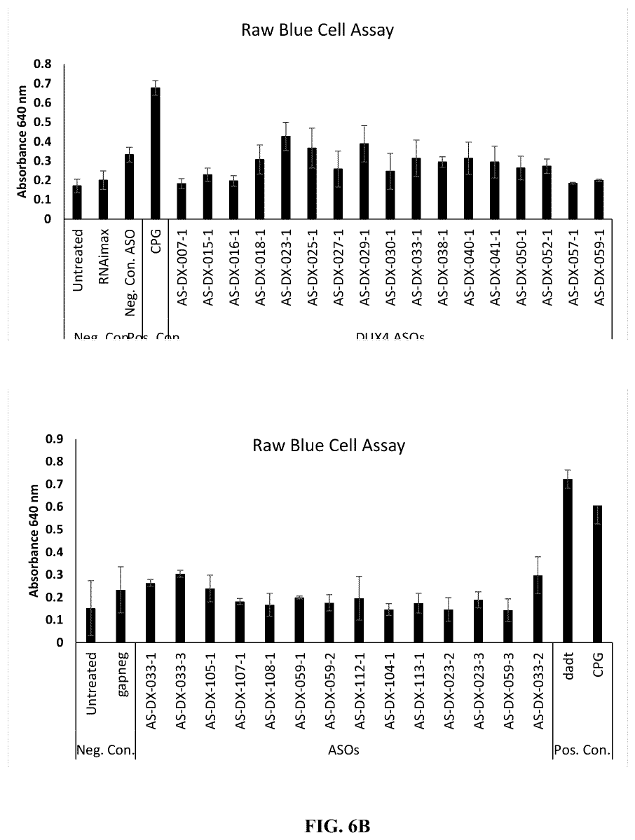

Referring to FIG. 6A, this figure shows reductions in innate immunostimulation for engineered DUX4 ASOs. Human Peripheral Blood Mononuclear Cells (PBMCs)(˜2-6×10⁵ cells) were plated in round bottom 96 well plates and transfected at 133 nM concentrations of the indicated ASOs for 48 hours with RNAiMAX reagent. Levels of IFN-α (Left axis) and TNF-α (right axis) in supernatant media were quantified by ELISA for 6 patients. Poly (dA:dT) (Pos Con #1) and an inmmunostimulatory oligonucleotide (Pos Con #2) served as positive controls for immunostimulation. RNAiMAX without oligonucleotide served as baseline negative control (Baseline) while transfection with a non-immunostimulatory RNA that does not target DUX4 (Neg Con) demonstrated low immunostimulation.

Referring to FIG. 6B,this figure further shows reductions in innate immunostimulation for engineered DUX4 ASOs through the Raw-Blue cell assay (Invivogen, raw-sp). In brief, cells were plated at 100,000 cells/well in 150 uL in a U bottom 96 well plate (Thermo, 163320) in DMEM (Thbermo, 11965092) with 10% FBS (Thermo, 10082147). After 24 hours, 22.34 uL of OptiMEM (Thermo, 31985088) is mixed with 2.66 uL of 10 uM ASO (per well), and 1 uL of Lipofectamine (per well). Lipofectamine/ASO mixtures are then added to each well of Raw Cells to make a final ASO concentration of 133 nM. Poly(dA:dT) (Invivogen, tlrl-patn) @ 1-10 ng/mL, CpG (Invivogen, tlrl-1585) @ 133 nM are used as positive controls. Cells are incubated for 1 day @ 37 degrees/5% CO2 with the transfection/ASO mixture. After incubation, gently spin the plate at 300×g for 5 min then collect 20 uL from each well and add to a new 96 well plate, flat bottom (VWR, 29442-056). Add 180 uL of QUANTI-Blue (Invivogen, rep-qbs) to each well of supernatant and incubate for 30m-6 hours @ 37 degrees/5% CO2. Read absorbance at 620-655 nm using Cytation 5 (Biotek). Data represents the mean of six replicate wells and error bars represent standard deviation.

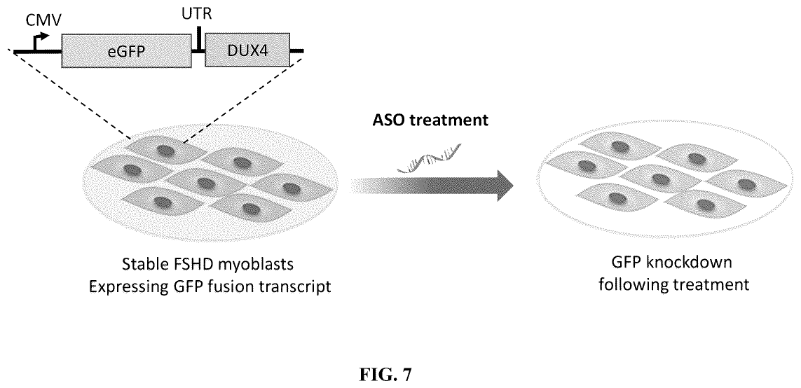

Referring to FIG. 7, DUX4 ASO HTS Assay Design. Stable human or mouse myoblasts expressing eGFP with the coding sequence for DUX4 in the 3′ UTR. Constitutive expression of this construct is driven by CMV for strong, ubiquitous expression. Unmolested mRNA encoding the eGFP-UTR-DUX4 transcript is transcribed and the eGFP sequence is translated. Translation of the toxic DUX4 protein is prevented by a stop codon at the end of the eGFP sequence, and mutation of the start codon for DUX4. ASOs that efficiently target DUX4 may bind to the fusion transcript and induce degradation through RNAse H or RISC, preventing GFP protein expression. Following treatment, a reduction of fluorescence may be observed in untreated cells and negative control transfection compared to experimental ASOs as assayed by plate reader or image analysis to efficiently generate reproducible results comparing the efficacy of DUX4 targeted ASOs. Two reporter assays, one in the immortalized mouse myoblast line C2C12 and another in the immortalized human FSHD myoblast line 15Abic were developed.

Referring to Table 6, this table display knockdown of a stable DUX4 GFP reporter screening assay. In black wall, clear bottom 96 well plates 10,000 15Abic stables or 1500 C2C12 stable cells are plated in their respective media. The following day after attachment, cells are then transfected using Lipofectamine™ RNAiMAX Transfection Reagent (13778075, Thermo Fisher Scientific). For each well 0.20 μL/well of Lipofectamine® RNAiMAX was mixed with 5 μL of Opti-MEM and incubated for 5 minutes. Then an equal volume of 20× ASO in Opti-MEM is added such that the total volume of the two transfection mixtures was 10 μL/well and such that the final concentration of ASO in a total well volume of 200 μL is 12.5 nM for c2c12 cells or 25 nM for 15Abic cells. The ASO-Opti-MEM mixture was incubated at room temperature for 15 mins. 10 μL of the resultant transfection reagent mixture was then added to each experimental well. After 6 hrs normal cell culture media is added to each well and then plates were incubated for 72-96 h at 37° C. The media was then replaced with 50 μL of FluoroBrite DMEM media (A1896701, Thermo Fisher Scientific) supplemented with L-glutamine and sodium pyruvate to 4 mM and 1 mM respectively for reading. Fluorescence intensity for each well was measured at 390+10 nm Excitation and 510+10 nm Emission on a Cytation 5 Cell Imaging Multi-Mode Reader (Biotek Instruments). Following fluorescence measurement the serum free FluoroBrite DMEM was removed and 100 μL of normal media was added to each well and WST-8 was used to measure cell viability/cell count. Cell viability measurements followed the manufacturers protocol. Briefly, 10 μL of WST-8 (ab228554, Abcam) was subsequently added to each well and the plate was oscillated to distribute the reagent evenly. Plates were then returned to the incubator for 30 mins to 3 hours depending on the cell density and cell type. To measure cell viability absorbance at 460 nm was measured on a Cytation 5. GFP measurements for each well are normalized to the WST-8 cell count. Values in the table represent mean GFP expression from six replicate wells and are displayed as a fraction of treatment with a negative control ASO.

TABLE 6
Knockdown of GFP-DUX4 reporter
with Oligonucleotide Therapies.

| | |
|---|---|
| C2C12 GFP Reporter Assay | 15Abic GFP Reporter Assay |

| ASO Name | Ave. FC | ASO Name | Ave. FC |
|---|---|---|---|
| Neg. Con | 0.99 | Neg. Con | 1.00 |
| AS-DX-001-1 | 0.86 | AS-DX-015-1 | 0.81 |
| AS-DX-002-1 | 0.78 | AS-DX-015-3 | 0.62 |
| AS-DX-003-1 | 0.93 | AS-DX-018-1 | 0.65 |
| AS-DX-004-1 | 0.86 | AS-DX-028-1 | 0.77 |
| AS-DX-005-1 | 0.88 | AS-DX-029-1 | 0.65 |
| AS-DX-006-1 | 0.96 | AS-DX-030-1 | 0.63 |
| AS-DX-008-1 | 0.84 | AS-DX-033-1 | 0.54 |
| AS-DX-009-1 | 0.70 | AS-DX-050-1 | 0.81 |
| AS-DX-010-1 | 0.59 | AS-DX-052-1 | 0.71 |
| AS-DX-011-1 | 0.54 | AS-DX-059-1 | 0.39 |
| AS-DX-012-1 | 0.67 | AS-DX-102-1 | 0.80 |
| AS-DX-015-1 | 0.66 | AS-DX-094-1 | 0.60 |
| AS-DX-015-3 | 0.41 | AS-DX-104-1 | 0.37 |
| AS-DX-018-1 | 0.54 | AS-DX-033-2 | 0.82 |
| AS-DX-019-1 | 0.84 | AS-DX-033-3 | 0.70 |
| AS-DX-021-1 | 0.99 | AS-DX-105-1 | 0.83 |
| AS-DX-022-1 | 0.85 | AS-DX-106-1 | 0.84 |
| AS-DX-023-1 | 0.53 | AS-DX-059-2 | 0.39 |
| AS-DX-025-1 | 0.57 | AS-DX-112-1 | 0.47 |
| AS-DX-026-1 | 0.55 | AS-DX-114-1 | 0.77 |
| AS-DX-027-1 | 0.33 | AS-DX-116-1 | 0.69 |
| AS-DX-028-1 | 0.55 | AS-DX-117-1 | 0.70 |
| AS-DX-029-1 | 0.53 | AS-DX-118-1 | 0.79 |
| AS-DX-030-1 | 0.56 | AS-DX-119-1 | 0.64 |
| AS-DX-032-1 | 0.80 | AS-DX-120-1 | 0.78 |
| AS-DX-033-1 | 0.53 | AS-DX-122-1 | 0.53 |
| AS-DX-034-1 | 0.89 | AS-DX-123-1 | 0.40 |
| AS-DX-035-1 | 0.87 | AS-DX-124-1 | 0.44 |
| AS-DX-036-1 | 1.03 | AS-DX-125-1 | 0.41 |
| AS-DX-037-1 | 0.87 | AS-DX-126-1 | 0.62 |
| AS-DX-038-1 | 0.53 | AS-DX-031-1 | 0.37 |
| AS-DX-040-1 | 0.48 | AS-DX-128-1 | 0.42 |
| AS-DX-041-1 | 0.43 | AS-DX-129-1 | 0.60 |
| AS-DX-043-1 | 0.83 | AS-DX-130-1 | 0.51 |
| AS-DX-044-1 | 0.75 | AS-DX-131-1 | 0.96 |
| AS-DX-045-1 | 0.59 | AS-DX-132-1 | 0.86 |
| AS-DX-046-1 | 0.97 | AS-DX-133-1 | 0.45 |
| AS-DX-018-2 | 0.82 | AS-DX-133-2 | 0.40 |
| AS-DX-048-1 | 0.84 | AS-DX-134-2 | 0.88 |
| AS-DX-049-1 | 0.89 | AS-DX-135-1 | 0.80 |
| AS-DX-050-1 | 0.98 | AS-DX-136-1 | 0.61 |
| AS-DX-051-1 | 0.66 | AS-DX-137-1 | 0.69 |
| AS-DX-052-1 | 0.59 | AS-DX-045-2 | 0.63 |
| AS-DX-054-1 | 0.86 | AS-DX-138-1 | 0.67 |
| AS-DX-055-1 | 0.87 | AS-DX-139-1 | 0.66 |
| AS-DX-057-1 | 0.67 | AS-DX-106-2 | 0.71 |
| AS-DX-058-1 | 0.72 | AS-DX-140-1 | 0.79 |
| AS-DX-059-1 | 0.27 | MRC-2107 | 0.64 |
| AS-DX-023-2 | 0.60 | | |
| AS-DX-023-3 | 0.39 | | |
| AS-DX-098-1 | 0.62 | | |
| AS-DX-099-1 | 0.55 | | |
| AS-DX-027-2 | 0.55 | | |
| AS-DX-027-3 | 0.68 | | |
| AS-DX-027-4 | 0.56 | | |
| AS-DX-100-1 | 0.50 | | |
| AS-DX-094-1 | 0.44 | | |
| AS-DX-104-1 | 0.34 | | |
| AS-DX-033-2 | 0.42 | | |
| AS-DX-033-3 | 0.18 | | |
| AS-DX-105-1 | 0.29 | | |
| AS-DX-106-1 | 0.50 | | |
| AS-DX-107-1 | 0.30 | | |
| AS-DX-108-1 | 0.23 | | |
| AS-DX-059-2 | 0.50 | | |
| AS-DX-112-1 | 0.50 | | |
| AS-DX-113-1 | 0.58 | | |
| AS-DX-059-3 | 0.20 | | |
| AS-DX-116-1 | 0.90 | | |
| AS-DX-117-1 | 0.89 | | |
| AS-DX-118-1 | 0.94 | | |
| AS-DX-119-1 | 0.63 | | |
| AS-DX-120-1 | 0.78 | | |
| AS-DX-128-1 | 0.62 | | |
| AS-DX-129-1 | 0.70 | | |
| AS-DX-130-1 | 0.94 | | |
| AS-DX-131-1 | 0.84 | | |
| AS-DX-132-1 | 0.63 | | |
| AS-DX-133-1 | 0.56 | | |
| AS-DX-133-2 | 0.75 | | |
| AS-DX-134-1 | 0.74 | | |
| AS-DX-134-2 | 0.77 | | |
| AS-DX-135-1 | 0.88 | | |
| AS-DX-137-1 | 0.52 | | |
| AS-DX-045-2 | 0.91 | | |
| AS-DX-139-1 | 0.76 | | |
| AS-DX-106-2 | 0.82 | | |
| AS-DX-140-1 | 0.53 | | |
| AS-DX-033-4 | 0.69 | | |
| AS-DX-141-1 | 0.59 | | |
| AS-DX-112-2 | 0.54 | | |
| AS-DX-142-1 | 0.72 | | |
| MRC-2107 | 0.71 | | |

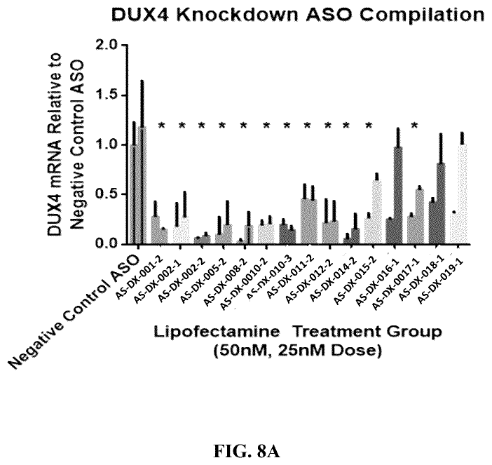

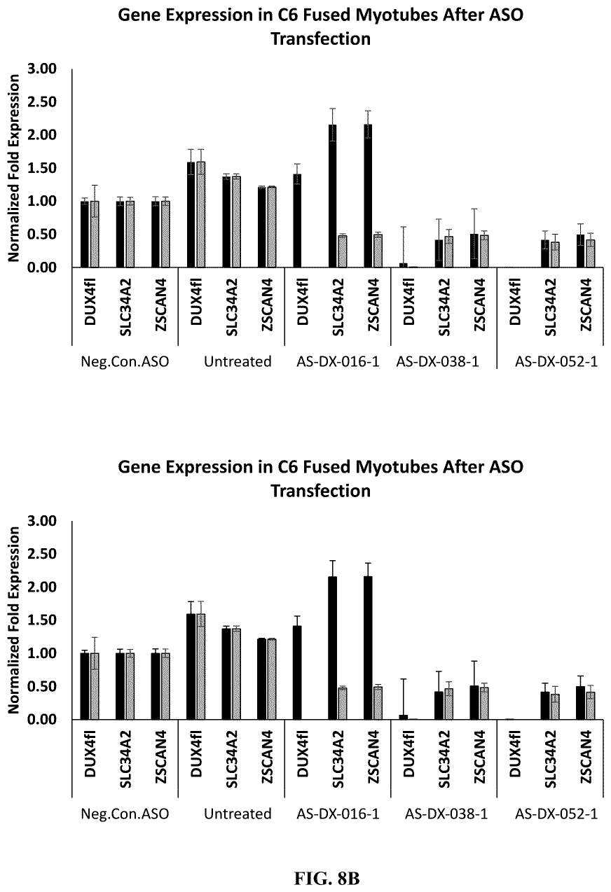

Referring to FIG. 8B and Table 7 displaying knockdown of DUX4 and DUX4 induced genes ZSCAN4 and SLC34A2 in FSHD myoblasts. 15Abic or C6 cells were plated in human Myogenic Precursor Cell (hMPC) media at density of 150,000 cells per well in twelve 24 well cell culture plates. hMPC media is composed of 500 mL RoosterBasal™-MSC, 10 mL RoosterBooster™-MSC (KT-001, RoosterBio), 91 mL of fetal bovine serum (10082147, Thermo Fisher), and 50 mL of 100 mM sodium pyruvate (11360070, Thermo Fisher). Three days after plating cells in hMPC media the media was replaced with hMPC Differentiation media which is composed of 500 mL RoosterBasal™-MSC, 10 mL RoosterBooster™-MSC, 11.4 mL of horse serum (16050130, Thermo Fisher), and 50 mL of 100 mM sodium pyruvate. Ten days after plating cells in hMPC media (seven days after plating cells in hMPC Differentiation media) cells were forward transfected using 0.875 μL of Lipofectamine™ RNAiMAX Transfection Reagent (13778075, Thermo Fisher) and 3.125 nM or 6.5 nM ASO in a well volume of 1 mL according to the manufacturer's directions. Five days after transfection cells were lysed into 350 μL of Buffer RLT (79216, Qiagen). RNA was extracted using Direct-zol-96 RNA Kit (R2056, Zymo Research) following manufacturer's directions. Purified RNA concentration was determined using a NanoDrop 1000 (Thermo Fisher) following manufacturer's directions. cDNA was generated by following the manufacturer's directions for the SuperScript™ IV First-Strand Synthesis System with ezDNase™ Enzyme kit (18091150, Thermo Fisher) with the following modifications. 1 μL of 110 μM dithiothreitol was added after digestion of genomic DNA. 1 μL of 50 μM anchored Oligo d(T)20 was used as the primer during reverse transcription. Following cDNA generation qPCR was performed to quantify three target genes: DUX4-fl (DUX4-full length), SLC34A2, and ZSCAN4 as well as one control gene: RPL13A. A multiplexed, probe-based qPCR reaction was done using dual quenched PrimeTime qPCR probes and primers (a forward primer, reverse primer, and probe constitute an assay). Briefly, in each well of a 96 well plate 10 μL of PrimeTime® Gene Expression Master Mix (1055772, Integrated DNA Technologies) was mixed with 100 ng of cDNA and 0.25 μL of each 20× assay (DUX4-fl, SLC34A2, ZSCAN4, RPL 13A) along with a volume of water such that the total volume of each was 20 μL. Thermal cycling and plate reading were done using a LightCycler 96 (Roche Diagnostics). Cycling conditions were as follows: polymerase activation at 95° C. for 180 seconds, denaturation at 95° C. for 15 seconds, and annealing/extension at 60° C. for 60 seconds with the denaturation and annealing/extension steps repeated for 40 cycles. Fluorescence was read at the end of the annealing/extension step after each cycle. Cycle threshold was automatically determined using the LightCycler 96 software. The normalized relative expression of the target genes was calculated following the method described by Taylor et al., 2019 (“The Ultimate qPCR Experiment: Producing Publication Quality, Reproducible Data the First Time”). Expression of the three target genes was then added together and a bar chart was produced describing the normalized, relative, composite knockdown of target genes in relation to a negative control ASO.

TABLE 7
qRT-PCR for Knockdown of DUX4 and DUX4 regulated Genes ZSCAN4 and
SLC34A2 following 3.125 nM treatment with DUX4 targeted ODN.

| | DUX4fl | DUX4fl | | ZSCAN4 | ZSCAN4 | SLC34A2 | SLC34A2 |
|---|---|---|---|---|---|---|---|
| ASO Name | Expression | Error | ASO Name | Expression | Error | Expression | Error |
| Neg. Con. ASO | 1.00 | 0.66 | Neg. Con. ASO | 1.00 | 0.41 | 1.00 | 0.75 |
| AS-DX-001-1 | 0.03 | 0.01 | AS-DX-002-1 | 0.83 | 0.09 | 0.51 | 0.14 |
| AS-DX-002-1 | 0.24 | 0.19 | AS-DX-008-1 | 1.11 | 0.24 | 0.65 | 0.29 |
| AS-DX-003-1 | 0.05 | 0.01 | AS-DX-015-1 | 0.93 | 0.09 | 0.07 | 0.12 |
| AS-DX-004-1 | 0.32 | 0.25 | AS-DX-015-3 | 0.77 | 0.13 | 0.72 | 0.41 |
| AS-DX-005-1 | 0.06 | 0.00 | AS-DX-018-1 | 1.11 | 0.04 | 0.86 | 0.44 |
| AS-DX-006-1 | 0.15 | 0.09 | AS-DX-025-1 | 2.08 | 0.28 | 0.30 | 0.08 |
| AS-DX-007-1 | 0.14 | 0.09 | AS-DX-028-1 | 1.31 | 0.13 | 0.42 | 0.25 |
| AS-DX-008-1 | 0.10 | 0.05 | AS-DX-029-1 | 1.55 | 0.45 | 0.83 | 0.44 |
| AS-DX-010-1 | 0.19 | 0.16 | AS-DX-030-1 | 0.69 | 0.08 | 0.33 | 0.17 |
| AS-DX-011-1 | 0.22 | 0.17 | AS-DX-033-1 | 1.28 | 0.06 | 0.52 | 0.05 |
| AS-DX-012-1 | 0.18 | 0.12 | AS-DX-038-1 | 1.52 | 0.04 | 0.37 | 0.14 |
| AS-DX-015-1 | 0.28 | 0.17 | AS-DX-041-1 | 1.04 | 0.03 | 0.09 | 0.03 |
| AS-DX-015-3 | 0.04 | 0.00 | AS-DX-048-1 | 0.66 | 0.03 | 0.35 | 0.09 |
| AS-DX-018-1 | 0.05 | 0.00 | AS-DX-050-1 | 0.87 | 0.16 | 0.18 | 0.09 |
| AS-DX-019-1 | 0.03 | 0.00 | AS-DX-051-1 | 0.90 | 0.07 | 0.81 | 0.16 |
| AS-DX-021-1 | 0.87 | 0.87 | AS-DX-052-1 | 1.21 | 0.07 | 0.41 | 0.07 |
| AS-DX-022-1 | 0.18 | 0.14 | AS-DX-056-1 | 0.67 | 0.05 | 0.51 | 0.13 |
| AS-DX-023-1 | 0.21 | 0.17 | AS-DX-059-1 | 1.04 | 0.04 | 1.24 | 0.30 |
| AS-DX-025-1 | 0.07 | 0.01 | AS-DX-023-2 | 1.28 | 0.38 | 0.92 | 0.81 |
| AS-DX-026-1 | 0.07 | 0.01 | AS-DX-023-3 | 1.54 | 0.19 | 1.73 | 0.19 |
| AS-DX-027-1 | 0.05 | 0.00 | AS-DX-027-2 | 1.21 | 0.11 | 0.31 | 0.15 |
| AS-DX-028-1 | 0.22 | 0.18 | AS-DX-027-4 | 0.78 | 0.12 | 0.18 | 0.09 |
| AS-DX-029-1 | 0.06 | 0.00 | AS-DX-104-1 | 0.63 | 0.10 | 0.63 | 0.42 |
| AS-DX-030-1 | 0.04 | 0.00 | AS-DX-033-3 | 0.47 | 0.05 | 0.09 | 0.01 |
| AS-DX-032-1 | 0.13 | 0.01 | AS-DX-105-1 | 0.58 | 0.09 | 0.24 | 0.03 |
| AS-DX-033-1 | 0.11 | 0.03 | AS-DX-050-4 | 0.73 | 0.14 | 1.00 | 0.37 |
| AS-DX-034-1 | 0.09 | 0.03 | AS-DX-109-1 | 0.61 | 0.09 | 0.44 | 0.26 |
| AS-DX-035-1 | 0.12 | 0.03 | AS-DX-112-1 | 0.84 | 0.14 | 0.30 | 0.24 |
| AS-DX-036-1 | 0.04 | 0.01 | AS-DX-113-1 | 0.55 | 0.03 | 0.25 | 0.10 |
| AS-DX-037-1 | 0.06 | 0.00 | | | | | |
| AS-DX-038-1 | 0.06 | 0.00 | | | | | |
| AS-DX-040-1 | 0.06 | 0.01 | | | | | |
| AS-DX-041-1 | 0.07 | 0.00 | | | | | |
| AS-DX-043-1 | 0.09 | 0.00 | | | | | |
| AS-DX-044-1 | 0.07 | 0.00 | | | | | |
| AS-DX-045-1 | 0.07 | 0.01 | | | | | |
| AS-DX-046-1 | 0.06 | 0.00 | | | | | |
| AS-DX-018-2 | 0.22 | 0.06 | | | | | |
| AS-DX-048-1 | 0.12 | 0.02 | | | | | |
| AS-DX-049-1 | 0.08 | 0.00 | | | | | |
| AS-DX-050-1 | 0.08 | 0.02 | | | | | |
| AS-DX-051-1 | 0.07 | 0.01 | | | | | |
| AS-DX-052-1 | 0.07 | 0.00 | | | | | |
| AS-DX-053-1 | 0.18 | 0.08 | | | | | |
| AS-DX-054-1 | 0.08 | 0.01 | | | | | |
| AS-DX-055-1 | 0.08 | 0.00 | | | | | |
| AS-DX-056-1 | 0.08 | 0.02 | | | | | |
| AS-DX-057-1 | 0.09 | 0.02 | | | | | |
| AS-DX-058-1 | 0.06 | 0.01 | | | | | |
| AS-DX-059-1 | 0.06 | 0.00 | | | | | |
| AS-DX-023-2 | 0.05 | 0.00 | | | | | |
| AS-DX-023-3 | 0.20 | 0.19 | | | | | |
| AS-DX-098-1 | 0.06 | 0.00 | | | | | |
| AS-DX-027-2 | 0.07 | 0.01 | | | | | |
| AS-DX-027-3 | 0.08 | 0.00 | | | | | |
| AS-DX-027-4 | 0.23 | 0.17 | | | | | |
| AS-DX-100-1 | 0.40 | 0.30 | | | | | |
| AS-DX-101-1 | 0.27 | 0.22 | | | | | |
| AS-DX-094-1 | 0.17 | 0.09 | | | | | |
| AS-DX-104-1 | 0.06 | 0.02 | | | | | |
| AS-DX-033-2 | 0.15 | 0.09 | | | | | |
| AS-DX-033-3 | 0.03 | 0.00 | | | | | |
| AS-DX-105-1 | 0.14 | 0.08 | | | | | |
| AS-DX-106-1 | 0.16 | 0.07 | | | | | |
| AS-DX-107-1 | 0.06 | N/A | | | | | |
| AS-DX-108-1 | 0.09 | 0.01 | | | | | |
| AS-DX-050-2 | 0.06 | 0.02 | | | | | |
| AS-DX-050-3 | 0.25 | 0.28 | | | | | |
| AS-DX-050-4 | 0.05 | 0.00 | | | | | |
| AS-DX-109-1 | 0.05 | 0.00 | | | | | |
| AS-DX-007-3 | 0.12 | 0.04 | | | | | |
| AS-DX-111-1 | 0.08 | 0.02 | | | | | |
| AS-DX-059-2 | 0.06 | 0.02 | | | | | |
| AS-DX-112-1 | 0.08 | 0.02 | | | | | |
| AS-DX-113-1 | 0.33 | 0.22 | | | | | |

Referring to Table 8, this table displays LD-50 values in HepG2 Liver cells for DUX4 targeted ASOs. HEPG2 cells (HB-8065, ATCC, Manassas, VA) were grown in DMEM (10-013-CV, Corning Inc.) supplemented with 10% FBS (FBS, 16000044, Thermo Fisher Scientific) and 1× penicillin-streptomycin (15140122, Thermo Fisher Scientific) Cells were grown at 37° C. at 5% CO2 in a humidified incubator. In 96 well plates 5,000 cell are plated in 180 μL of media w/o antibiotics. Immediately following plating cells are transfected using Lipofectamine™ RNAiMAX Transfection Reagent (13778075, Thermo Fisher Scientific). For 100 nM transfection of ASO 0.4 IL/well of RNAiMAX is diluted into 10 μL of Opti-MEM and then combined with 10 μL of 1 μM ASO (10× final culture volume) in Opti-MEM and incubated at room temperature for 15 minutes. Lower doses of ASO are created by serial 1:2 dilution of 100 nM complexes. Higher concentrations are prepared by increasing the concentration of the ASO but maintaining 0.4 μL/well of RNAiMAX which is the highest dose that may be used without causing cytotoxicity. Twenty μL of appropriate diluted ASO/RNAiMAX complexes are then added to each well within 30 minutes of complex formation. Plates were gently oscillated to evenly distribute the transfection reagents in the well and were then returned to the incubator. Cells are treated for 72-96 h at 37° C. Following treatment transfection media is removed and 100 μL of fresh media is added to each well and WST-8 assay is performed to measure cell viability/cell count. Cell viability measurements followed the manufacturers protocol. Briefly, 10 μL of WST-8 (ab228554, Abcami) was added to each well and the plate was oscillated to distribute the reagent evenly. Plates were then returned to the incubator for 90 mins. Next absorbance at 460 nm was measured on a Cytation 5. Data was analyzed by subtracting the average background cell viability measurements of cell free wells (wells with only media and WST8 reagent in them) from wells containing cells. Cell viability is calculated by normalization to wells that are mock transfected with only Opti-mem. Lethal dose 50 (LD50) concentration values are extrapolated from dose curves using a custom excel macro designed for this purpose.

TABLE 8
Lethal Dose 50 values in HepG2 Liver
cells for DUX4 targeted ASOs.

| ASO Name | IC50 | ASO Name | IC50 |
|---|---|---|---|
| Neg. Con. ASO | >300 | AS-DX-059-1 | 283.17 |
| AS-DX-007-1 | >300 | AS-DX-027-2 | 38.66 |
| AS-DX-015-1 | >300 | AS-DX-027-4 | 38.91 |
| AS-DX-015-3 | 153.17 | AS-DX-100-1 | 96.77 |
| AS-DX-018-1 | 67.84 | AS-DX-033-2 | 177.65 |
| AS-DX-023-1 | 31.11 | AS-DX-033-3 | 52.43 |
| AS-DX-025-1 | 53.00 | AS-DX-105-1 | 51.96 |
| AS-DX-027-1 | 10.30 | AS-DX-106-1 | 60.38 |
| AS-DX-028-1 | 50.22 | AS-DX-107-1 | 88.52 |
| AS-DX-029-1 | 42.15 | AS-DX-108-1 | 112.89 |
| AS-DX-030-1 | >300 | AS-DX-050-2 | 113.08 |
| AS-DX-033-1 | 53.84 | AS-DX-050-3 | >300 |
| AS-DX-038-1 | >300 | AS-DX-050-4 | >300 |
| AS-DX-040-1 | 68.18 | AS-DX-109-1 | 105.82 |
| AS-DX-041-1 | 40.99 | AS-DX-007-3 | >300 |
| AS-DX-050-1 | 96.11 | AS-DX-111-1 | >300 |
| AS-DX-052-1 | >300 | AS-DX-059-2 | >300 |
| AS-DX-057-1 | 42.42 | AS-DX-112-1 | >300 |
| AS-DX-058-1 | 14.50 | AS-DX-059-3 | >300 |

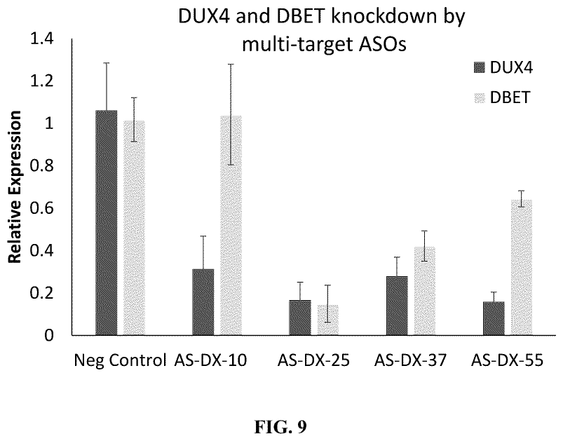

Referring to FIG. 9, this figure shows simultaneous knockdown of DUX4 and DBET RNA transcripts in FSHD patient myoblasts by multi-targeted antisense oligonucleotides (ASOs). AS-DX-10 only targets the DUX4 transcript, while AS-DX-25, -37, and -55 target both DUX4 and DBET transcripts. Immortalized 15Abic myoblast cells were plated in 12-well plates and the next day transfected with control or targeted ASOs at 50 nM using the transfection agent RNAiMAX. One day after plating differentiation media was added to induce myoblast formation and DUX4 expression. 72 hrs after transfection cells were lysed and total RNA was collected from the wells and RT-qPCR was performed to determine expression of DUX4 or DBET transcripts. ASOs AS-DX-25, -37, and -55 knockdown both DUX4 and DBET transcripts while AS-DX-10 only knocks down DUX4.

### Example 4: MC-DX4 Off-Target Analysis and Validation for ASO Target Sequences

### Identifying Off Target Transcripts

All potential reverse complement ASO positions in the DUX4 coding gene (ENSG00000258389.2), from 15 bp to 20 bp were generated with 1 bp sliding in the reference sequence across the DUX4 region chr4:190,173,774-190,185,942. A modified script of GGGenome (https://gggenome.dbcls.jp/) was used for rapid alignment of our oligonucleotide sequences to the human transcriptome (Human RNA Refseq release 205, March 2021). This script identified all transcripts that are partially complimentary to each possible ASOs targeting DUX4. We then analyze these hits with algorithms to identify higher likely off-targets. These may contain up to 3 mismatches, gaps, or bulges (WO2021203043), but they must obey a series of other principles related to structural conformation, affinity, and transcript expression. Even with these filters there are still many predicted off target transcripts that are likely false positives, or context dependent, and need to be validated through experimental testing in vitro and in vivo.

### Filtering Off-Target Interactions for Potential Positive FSHD Related Targets

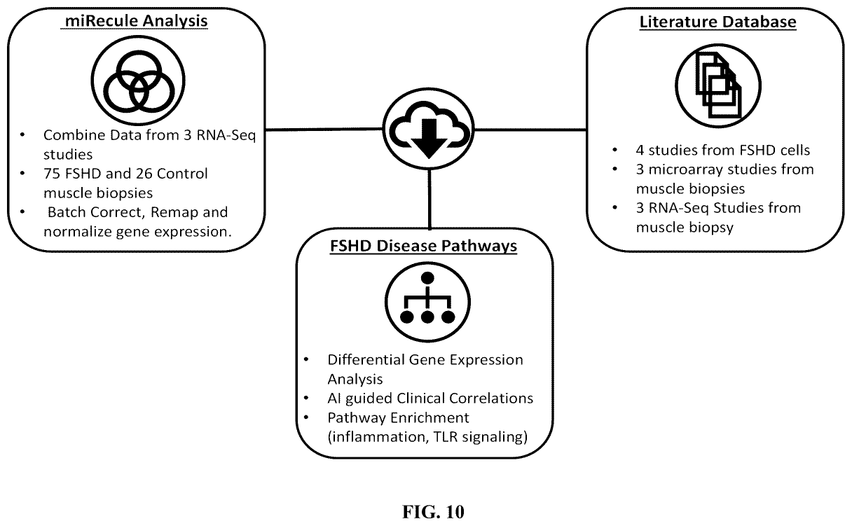

Patient segregation and gene expression analysis are critical parts of the disclosed data analysis strategy to understand disease biology. This starts with gathering available datasets from the literature reporting RNA expression patterns from muscle tissues and patient cells. The inventors assembled a database of 10 studies with rigorous standards for sample handling, transcriptomic profiling by microarray and RNAseq, and significant patient information. These 10 studies include: Genes with increased expression in myoblasts overexpressing DUX4 (Tsumagari et. al. 2011(31), Pakula et. al. 2013, (32) Geng et. al. 2012(33), and Mitsuhashi et. al. 2021 (34)); Microarray Studies for human muscle biopsies (Winokur et. al. 2003 (35), and Rahimov et. al. 2012 (36)); and RNA-seq profiles (Yao et al. 2014(28), Wong et al. 2020(17), and Wang et al. 2019 (29)). One drawback of these studies, is they often contain low patient numbers, lacking statistical power. To overcome this problem, the inventors created a new dataset from the three RNA-seq studies with available data to improve the statistical power, and ability to derive correlations with clinical attributes of the patients. FIG. 10 shows an overview our datasets and our analysis.

First, the inventors identified the genes that were commonly upregulated in FSHD muscle vs. control muscle among published datasets or using inventors' own RNA-seq analysis. The inventors also utilized principal component analysis and hierarchical clustering to segregate patients into groups and compared expression patterns between the control samples and these groups. Interestingly, the clusters align well with clinical severity scores (i.e., mild, moderate, or severe diseases). Supporting this analysis, similar results were obtained from a similar analysis from a subset of the samples included in the larger meta-analysis as displayed in FIG. 11. From this analysis the inventors created a database of upregulated genes in FSHD keeping with each gene the supporting evidence for this dysregulation, and any associated clinical correlations. From this database the inventors next performed pathway enrichment analysis utilizing GO pathway analysis (37). The top upregulated pathways include inflammatory response and other immune regulated pathways, cellular proliferation, cell cycle regulation, and fibrosis.

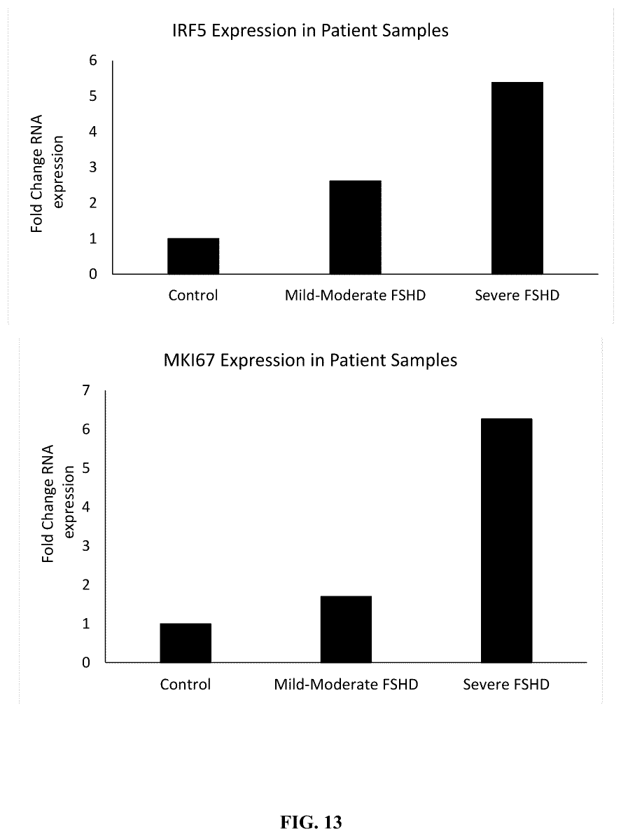

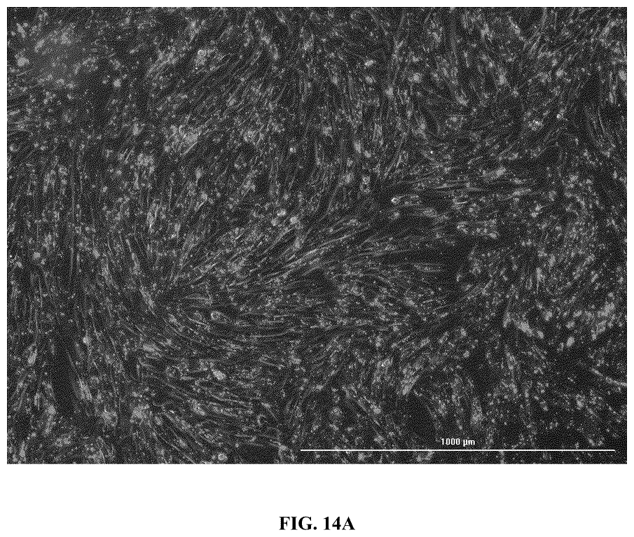

Having assembled this database of FSHD related genes and pathways, we then filtered our identified potential off-target interaction against this list. Potential off-target interactions that match an FSHD related gene, or co-targets, are displayed in the right most column in Table 2. For example, AS-DX-007 is predicted to target three co-targets associated with FSHD, DBET, MKI67, and IRF5. DBET is a non-coding RNA associated with opening of the D4Z4 repeats, and expression of DUX4 (38). MKI67 encodes the Ki-67 protein, which we detected in the upregulated FSHD muscle tissue, and may be involved in the DUX4 induction of the muscle fiber cell proliferation and damage (FIG. 12A). IRF5 (Interferon Regulatory Factor 5) encodes a transcription factor that is upregulated by several inflammatory signals, and results in the expression of several cytokines such as TNF, and induction of the intracellular interferon response (FIG. 12B). Our analysis also demonstrated increased expression of these genes in mild and severe FSHD (FIG. 13).

### Filtering Off-Target Interactions for Potential Negative Toxicity Related Interactions

To identify potential off-target interactions that may be associated with toxicity that may be desirable to avoid, the inventors utilized the Ingenuity knowledge database which accumulates peer reviewed publications, and toxicity related gene expression datasets from Tox net and other databases to associate off-target genes with potential toxicity. The inventors also identified genes related to muscle differentiation, development and function by go-pathway analysis. The inventors filtered oligonucleotide sequences identified off-target interactions for matches for IPAs Toxicity knowledge base or go pathways. For example, NR4A1 is associated with liver and kidney cell death and fibrosis, and muscle cell differentiation.

Validation of Co-Target Interaction by qRT-PCR

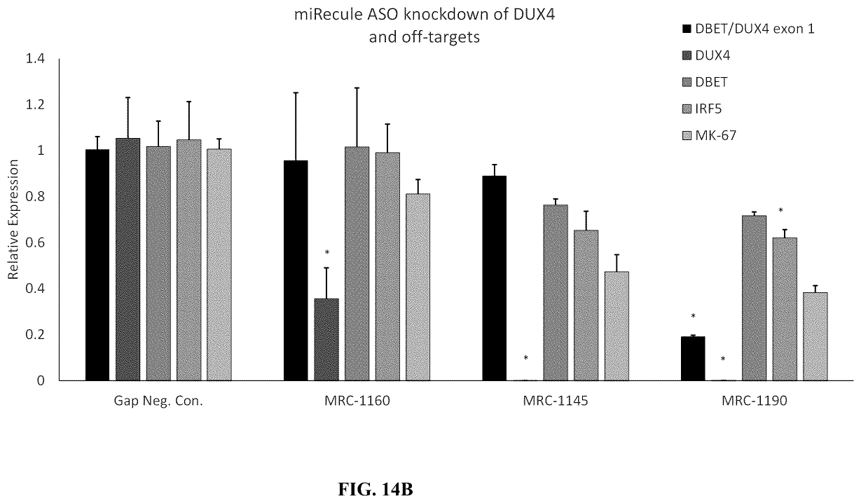

To validate off-target interactions, the FSHD myoblast line 15Abic was used. 2.5e5 15abic myoblasts were plated in 6-well plates. After 24 hours, replication media was removed, and 2 mL of differentiation media added and 250 μL of optimum containing appropriate ASO RNAimax complexes, so that the final concentration of each ASO was 50 nM. ASO treatments included fluorescent negative control ASO, AS-DX-015-1, which only targets DUX4 as a positive control, or AS-DX-007-1 or AS-DX-050-1 which may co-target DBET, IRF5, and MKI67. At the start of transfection, the differentiation media was added to the cells to induce fusion into myotubes and DUX4 expression. Referring to FIG. 14A displays near 100% transfection efficiency of the fluorescent ASO under optimized conditions 48 hours after transfection. After 96 hours of the transfection, myotube fusion was observed by cell morphology, and the total RNA was collected. cDNA was created, and qRT-PCR was performed for DUX4 and the co-target genes. Referring to FIG. 14B the graph displays the mean of three biological replicate wells, and error bars represent standard error of the mean. * denotes a p-value of <0.05 by 2-tailed student's t-test. Robust knockdown of DUX4 was observed for all ASOs, and significant knockdown of co-targets was observed with AS-DX-007-1 and AS-DX-050-1.

While preferred aspects of the present disclosure have been shown and described herein, such aspects are provided by way of example only. Numerous variations, changes, and substitutions may occur. It should be understood that various alternatives to the aspects of the disclosure described herein may be employed in practicing the disclosure. It is intended that the following claims define the scope of the disclosure and that methods and structures within the scope of these claims and their equivalents be covered thereby.

### REFERENCES

1. Hamel J, Tawil R. Facioscapulohumeral Muscular Dystrophy: Update on Pathogenesis and Future Treatments. Neurotherapeutics. 2018; 15(4):863-71. doi: 10.1007/s13311-018-00675-3. PubMed PMID: 30361930; PMCID: PMC6277282.
2. Do T N, Street N, Donnelly J, Adams M M, Cunniff C, Fox D J, Weinert R O, Oleszek J, Romitti P A, Westfield C P, Bolen J, Muscular Dystrophy Surveillance T, Research N. Muscular Dystrophy Surveillance, Tracking, and Research Network pilot: Population-based surveillance of major muscular dystrophies at four U.S. sites, 2007-2011. Birth Defects Res. 2018; 110(19):1404-11. doi: 10.1002/bdr2.1371. PubMed PMID: 30070776; PMCID: PMC6265066.
3. Richards M, Coppee F, Thomas N, Belayew A, Upadhyaya M. Facioscapulohumeral muscular dystrophy (FSHD): an enigma unravelled? Hum Genet. 2012; 131(3):325-40. doi: 10.1007/s00439-011-1100-z. PubMed PMID: 21984394.
4. Kowaljow V, Marcowycz A, Ansseau E, Conde C B, Sauvage S, Matteotti C, Arias C, Corona E D, Nunez N G, Leo O, Wattiez R, Figlewicz D, Laoudj-Chenivesse D, Belayew A, Coppee F, Rosa A L. The DUX4 gene at the FSHD1A locus encodes a pro-apoptotic protein. Neuromuscul Disord. 2007; 17(8):611-23. doi: 10.1016/j.nmd.2007.04.002. PubMed PMID: 17588759.
5. Celegato B, Capitanio D, Pescatori M, Romualdi C, Pacchioni B, Cagnin S, Vigano A, Colantoni L, Begum S, Ricci E, Wait R, Lanfranchi G, Gelfi C. Parallel protein and transcript profiles of FSHD patient muscles correlate to the D4Z4 arrangement and reveal a common impairment of slow to fast fibre differentiation and a general deregulation of MyoD-dependent genes. Proteomics. 2006; 6(19):5303-21. doi: 10.1002/pmic.200600056. PubMed PMID: 17013991.
6. A prospective, quantitative study of the natural history of facioscapulohumeral muscular dystrophy (FSHD): implications for therapeutic trials. The FSH-DY Group. Neurology. 1997; 48(1):38-46. doi: 10.1212/wnl.48.1.38. PubMed PMID: 9008491.
7. Tawil R, Forrester J, Griggs R C, Mendell J, Kissel J, McDermott M, King W, Weiffenbach B, Figlewicz D. Evidence for anticipation and association of deletion size with severity in facioscapulohumeral muscular dystrophy. The FSH-DY Group. Ann Neurol. 1996; 39(6):744-8. doi: 10.1002/ana.410390610. PubMed PMID: 8651646.
8. Mah J K, Chen Y W. A Pediatric Review of Facioscapulohumeral Muscular Dystrophy. J Pediatr Neurol. 2018; 16(4):222-31. doi: 10.1055/s-0037-1604197. PubMed PMID: 30923442; PMCID: PMC6435288.
9. Padberg G W, Brouwer O F, de Keizer R J, Dijkman G, Wijmenga C, Grote J J, Frants R R. On the significance of retinal vascular disease and hearing loss in facioscapulohumeral muscular dystrophy. Muscle Nerve Suppl. 1995(2):S73-80. PubMed PMID: 23573590.
10. Ansseau E, Vanderplanck C, Wauters A, Harper S Q, Coppee F, Belayew A. Antisense Oligonucleotides Used to Target the DUX4 mRNA as Therapeutic Approaches in FaciosScapuloHumeral Muscular Dystrophy (FSHD). Genes (Basel). 2017; 8(3). doi: 10.3390/genes8030093. PubMed PMID: 28273791; PMCID: PMC5368697.
11. Bao B, Maruyama R, Yokota T. Targeting mRNA for the treatment of facioscapulohumeral muscular dystrophy. Intractable Rare Dis Res. 2016; 5(3):168-76. doi: 10.5582/irdr.2016.01056. PubMed PMID: 27672539; PMCID: PMC4995414.
12. Wallace L M, Garwick S E, Mei W, Belayew A, Coppee F, Ladner K J, Guttridge D, Yang J, Harper S Q. DUX4, a candidate gene for facioscapulohumeral muscular dystrophy, causes p53-dependent myopathy in vivo. Ann Neurol. 2011; 69(3):540-52. doi: 10.1002/ana.22275. PubMed PMID: 21446026; PMCID: PMC4098764.
13. Chen J C, King O D, Zhang Y, Clayton N P, Spencer C, Wentworth B M, Emerson C P, Jr., Wagner K R. Morpholino-mediated Knockdown of DUX4 Toward Facioscapulohumeral Muscular Dystrophy Therapeutics. Mol Ther. 2016; 24(8):1405-11. doi: 10.1038/mt.2016.111. PubMed PMID: 27378237; PMCID: PMC5023379.
14. Wallace L M, Saad N Y, Pyne N K, Fowler A M, Eidahl J O, Domire J S, Griffin D A, Herman A C, Sahenk Z, Rodino-Klapac L R, Harper S Q. Pre-clinical Safety and Off-Target Studies to Support Translation of AAV-Mediated RNAi Therapy for FSHD. Mol Ther Methods Clin Dev. 2018; 8:121-30. doi: 10.1016/j.omtm.2017.12.005. PubMed PMID: 29387734; PMCID: PMC5787672.
15. Wallace L M, Liu J, Domire J S, Garwick-Coppens S E, Guckes S M, Mendell J R, Flanigan K M, Harper S Q. RNA interference inhibits DUX4-induced muscle toxicity in vivo: implications for a targeted FSHD therapy. Mol Ther. 2012; 20(7):1417-23. doi: 10.1038/mt.2012.68. PubMed PMID: 22508491; PMCID: PMC3392971.
16. Pandey S N, Lee Y C, Yokota T, Chen Y W. Morpholino treatment improves muscle function and pathology of Pitx1 transgenic mice. Mol Ther. 2014; 22(2):390-6. doi: 10.1038/mt.2013.263. PubMed PMID: 24232919; PMCID: PMC3916049.
17. Wong C J, Wang L H, Friedman S D, Shaw D, Campbell A E, Budech C B, Lewis L M, Lemmers R, Statland J M, van der Maarel S M, Tawil R N, Tapscott S J. Longitudinal measures of RNA expression and disease activity in FSHD muscle biopsies. Hum Mol Genet. 2020; 29(6):1030-43. doi: 10.1093/hmg/ddaa031. PubMed PMID: 32083293; PMCID: PMC7158378.
18. Xie S, Xiang Y, Wang X, Ren H, Yin T, Ren J, Liu W. Acquired cholesteatoma epithelial hyperproliferation: Roles of cell proliferation signal pathways. Laryngoscope. 2016; 126(8):1923-30. doi: 10.1002/lary.25834. PubMed PMID: 26989841.
19. Elkon K B, Briggs T A. The (Orf)ull truth about IRF5 and type I interferons in SLE. Nat Rev Rheumatol. 2020; 16(10):543-4. doi: 10.1038/s41584-020-0472-7. PubMed PMID: 32709997.
20. S.T. C. Antisense Drug Technology: Principles, Strategies, and Applications, Second Edition 2008.
21. Reinert K, Langmead B, Weese D, Evers D J. Alignment of Next-Generation Sequencing Reads. Annu Rev Genomics Hum Genet. 2015; 16:133-51. doi: 10.1 146/annurev-genom-090413-025358. PubMed PMID: 25939052.
22. van der Maarel S M, Frants R R. The D4Z4 repeat-mediated pathogenesis of facioscapulohumeral muscular dystrophy. Am J Hum Genet. 2005; 76(3):375-86. doi: 10.1086/428361. PubMed PMID: 15674778; PMCID: PMC1 196390.
23. Ballarati L, Piccini I, Carbone L, Archidiacono N, Rollier A, Marozzi A, Meneveri R, Ginelli E. Human genome dispersal and evolution of 4q35 duplications and interspersed LSau repeats. Gene. 2002; 296(1-2):21-7. doi: 10.1016/s0378-1119(02)00858-2. PubMed PMID: 12383499.
24. Das S, Chadwick B P. Influence of Repressive Histone and DNA Methylation upon D4Z4 Transcription in Non-Myogenic Cells. PLoS One. 2016; 11(7):e0160022. doi: 10.1371/journal.pone.0160022. PubMed PMID: 27467759; PMCID: PMC4965136.
25. Snider L, Asawachaichan A, Tyler A E, Geng L N, Petek L M, Maves L, Miller D G, Lemmers R J, Winokur S T, Tawil R, van der Maarel S M, Filippova G N, Tapscott S J. RNA transcripts, miRNA-sized fragments and proteins produced from D4Z4 units: new candidates for the pathophysiology of facioscapulohumeral dystrophy. Hum Mol Genet. 2009; 18(13):2414-30. doi: 10.1093/hmg/ddp180. PubMed PMID: 19359275; PMCID: PMC2694690.
26. Hagedom P H, Pontoppidan M, Bisgaard T S, Berrera M, Dieckmann A, Ebeling M, Moller M R, Hudlebusch H, Jensen M L, Hansen H F, Koch T, Lindow M. Identifying and avoiding off-target effects of RNase H-dependent antisense oligonucleotides in mice. Nucleic Acids Res. 2018; 46(11):5366-80. doi: 10.1093/nar/gky397. PubMed PMID: 29790953; PMCID: PMC6009603.
27. Scharner J, Ma W K, Zhang Q, Lin K T, Rigo F, Bennett C F, Krainer A R. Hybridization-mediated off-target effects of splice-switching antisense oligonucleotides. Nucleic Acids Res. 2020; 48(2):802-16. doi: 10.1093/nar/gkz1132. PubMed PMID: 31802121; PMCID: PMC6954394.
28. Yao Z, Snider L, Balog J, Lemmers R J, Van Der Maarel S M, Tawil R, Tapscott S J. DUX4-induced gene expression is the major molecular signature in FSHD skeletal muscle. Hum Mol Genet. 2014; 23(20):5342-52. doi: 10.1093/hmg/ddu251. PubMed PMID: 24861551; PMCID: PMC4168822.
29. Wang L H, Friedman S D, Shaw D, Snider L, Wong C J, Budech C B, Poliachik S L, Gove N E, Lewis L M, Campbell A E, Lemmers R, Maarel S M, Tapscott S J, Tawil R N. MRI-informed muscle biopsies correlate MRI with pathology and DUX4 target gene expression in FSHD. Hum Mol Genet. 2019; 28(3):476-86. doi: 10.1093/hmg/ddy364. PubMed PMID: 30312408; PMCID: PMC6337697.
30. Consortium G T. The Genotype-Tissue Expression (GTEx) project. Nat Genet. 2013; 45(6):580-5. doi: 10.1038/ng.2653. PubMed PMID: 23715323; PMCID: PMC4010069.
31. Tsumagari K, Chang S C, Lacey M, Baribault C, Chittur S V, Sowden J, Tawil R, Crawford G E, Ehrlich M. Gene expression during normal and FSHD myogenesis. BMC Med Genomics. 2011; 4:67. doi: 10.1186/1755-8794-4-67. PubMed PMID: 21951698; PMCID: PMC3204225.
32. Pakula A, Schneider J, Janke J, Zacharias U, Schulz H, Hubner N, Mahler A, Spuler A, Spuler S, Carlier P, Boschmann M. Altered expression of cyclin A 1 in muscle of patients with facioscapulohumeral muscle dystrophy (FSHD-1). PLoS One. 2013; 8(9):e73573. doi: 10.1371/journal.pone.0073573. PubMed PMID: 24019929; PMCID: PMC3760810.
33. Geng L N, Yao Z, Snider L, Fong A P, Cech J N, Young J M, van der Maarel S M, Ruzzo W L, Gentleman R C, Tawil R, Tapscott S J. DUX4 activates germline genes, retroelements, and immune mediators: implications for facioscapulohumeral dystrophy. Dev Cell. 2012; 22(1):38-51. doi: 10.1016/j.devce1.2011.11.013. PubMed PMID: 22209328; PMCID: PMC3264808.
34. Mitsuhashi S, Nakagawa S, Sasaki-Honda M, Sakurai H, Frith M C, Mitsuhashi H. Nanopore direct RNA sequencing detects DUX4-activated repeats and isoforms in human muscle cells. Hum Mol Genet. 2021; 30(7):552-63. doi: 10.1093/hmg/ddab063. PubMed PMID: 33693705; PMCID: PMC8120133.
35. Winokur S T, Chen Y W, Masny P S, Martin J H, Ehmsen J T, Tapscott S J, van der Maarel S M, Hayashi Y, Flanigan K M. Expression profiling of FSHD muscle supports a defect in specific stages of myogenic differentiation. Hum Mol Genet. 2003; 12(22):2895-907. doi: 10.1093/hmg/ddg327. PubMed PMID: 14519683.
36. Rahimov F, King O D, Leung D G, Bibat G M, Emerson C P, Jr., Kunkel L M, Wagner K R. Transcriptional profiling in facioscapulohumeral muscular dystrophy to identify candidate biomarkers. Proc Natl Acad Sci USA. 2012; 109(40):16234-9. doi: 10.1073/pnas.1209508109. PubMed PMID: 22988124; PMCID: PMC3479603.
37. Gene Ontology C. Gene Ontology Consortium: going forward. Nucleic Acids Res. 2015; 43(Database issue):D1049-56. doi: 10.1093/nar/gku1179. PubMed PMID: 25428369; PMCID: PMC4383973.
38. Cabianca D S, Casa V, Bodega B, Xynos A, Ginelli E, Tanaka Y, Gabellini D. A long ncRNA links copy number variation to a polycomb/trithorax epigenetic switch in FSHD muscular dystrophy. Cell. 2012; 149(4):819-31. doi: 10.1016/j.cell.2012.03.035. PubMed PMID: 22541069; PMCID: PMC3350859.
39. Untergasser A, Cutcutache I, Koressaar T, Ye J, Faircloth B C, Remm M, Rozen S G. Primer3—new capabilities and interfaces. Nucleic Acids Res. 2012 August; 40(15):e115. doi: 10.1093/nar/gks596. Epub 2012 Jun. 22. PMID: 22730293; PMCID: PMC3424584.
40. SantaLucia J Jr. A unified view of polymer, dumbbell, and oligonucleotide DNA nearest-neighbor thermodynamics. Proc Natl Acad Sci USA. 1998 Feb. 17; 95(4):1460-5. doi: 10.1073/pnas.95.4.1460. PMID: 9465037; PMCID: PMC19045.
41. Owczarzy R, You Y, Moreira B G, Manthey J A, Huang L, Behlke M A, Walder J A. Effects of sodium ions on DNA duplex oligomers: improved predictions of melting temperatures. Biochemistry. 2004 Mar. 30; 43(12):3537-54. doi: 10.1021/bi034621r. PMID: 15035624.

1. An engineered DUX4-targeting oligonucleotide that is from about 15 to about 25 nucleotides in length, wherein the engineered DUX4-targeting oligonucleotide comprises at least about: 80%, 85%, 90%, or 95% sequence identity to any one of SEQ. ID. NOs: 20,962-42,138.

2. The engineered DUX4-targeting oligonucleotide of claim 1, that is from about 15 to about 25 nucleotides in length, wherein the engineered DUX4-targeting oligonucleotide comprises at least about 80%, 85%, 90%, or 95% sequence identity to any one of SEQ. ID. NOs: 42,006-42,138.

3. The engineered DUX4-targeting oligonucleotide of claim 1, that is complementary to a binding site in a DUX4 RNA that is greater than 85% conserved among individuals.

4. The engineered DUX4-targeting oligonucleotide of claim 2, wherein the engineered DUX4-targeting oligonucleotide comprises a DNA nucleotide and an RNA nucleotide.

5. The engineered DUX4-targeting oligonucleotide of claim 1, wherein the oligonucleotide comprises a DNA nucleotide, and/or an RNA nucleotide, optionally wherein the engineered DUX4-targeting oligonucleotide is small interfering RNA (siRNA), a MicroRNA (miRNA), a small nuclear RNA (snRNA), a U spliceosomal RNA (U-RNA), a Small nucleolar RNA (snoRNA), a Piwi-interacting RNA (piRNA), a repeat associated small interfering RNA (rasiRNA), a small rDNA-derived RNA (srRNA), a transfer RNA derived small RNA (tsRNA), a ribosomal RNA derived small RNA (rsRNA), a large non-coding RNA derived small RNA (lncsRNA), or a messenger RNA derived small RNA (msRNA) an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), or an exon skipping oligonucleotide.

6-7. (canceled)

8. The engineered DUX4-targeting oligonucleotide of claim 1, wherein the engineered DUX4-targeting oligonucleotide comprises at least one nucleobase selected from the list consisting of a locked nucleic acid nucleobase, a 2′Omethyl nucleobase, or a 2′Methoxyethyl nucleobase.

9. The engineered DUX4-targeting oligonucleotide of claim 2, which binds to the DUX4 coding sequence in an aqueous solution with a predicted melting temperature (Tm) from about 45 to about 65 degrees Celsius wherein the aqueous solution has a pH ranging of from about 7.2 to about 7.6.

10. A conjugate comprising i) the engineered DUX4-targeting oligonucleotide of claim 1; ii) an antibody, an antibody fragment, a single monomeric variable antibody domain, a naturally occurring ligand, a small molecule, or a peptide; and optionally iii) a linker that links i) to ii).

11. A vector containing or encoding the engineered DUX4-targeting oligonucleotide of claim 1.

12-16. (canceled)

17. A pharmaceutical composition comprising the engineered DUX4-targeting oligonucleotide of claim 1, and a pharmaceutically acceptable: excipient, diluent, carrier, or a combination thereof.

18-20. (canceled)

21. A kit comprising the engineered DUX4-targeting oligonucleotide of claim 1.

22. (canceled)

23. A method of treating a disease or condition in a subject comprising administering to the subject a therapeutically effective amount the pharmaceutical composition of claim 17.

24. The method of claim 23, wherein the disease or condition is a DUX4 mediated disease or condition, optionally wherein the DUX4 mediated disease or condition is facioscapulohumeral muscular dystrophy.

25-33. (canceled)

34. The method of claim 23, further comprising concurrently or consecutively administering a co-therapy.

35. A method comprising administering the engineered DUX-4 targeting oligonucleotide of claim 1 to a subject, wherein after the administering, the engineered DUX-4 targeting oligonucleotide selectively hybridizes to two different endogenous disease related RNAs wherein one of the two different endogenous disease related RNAs is a DUX4 RNA transcribed from a first genetic loci and one of the two different endogenous disease related RNAs is transcribed from a different genetic loci than the first genetic loci.

36. The method of claim 35, wherein the second of the two different endogenous disease related RNAs is selected from SEQ ID NOs: 42139-42894

37. The method of claim 35, wherein the engineered DUX4-targeting oligonucleotide hybridizes to the endogenous disease related RNA that is transcribed from a different genetic loci than the first genetic loci, such that upon hybridization there are no more than 4 mismatches, bulges, insertions or deletions in the binding site, and the resulting duplex contains two regions of complementarity at least 7 contiguous nucleobases long, or one region at least 10 contiguous nucleobases long.

38. The method of claim 35, wherein the method is a method of treating a disease or condition which is a DUX4 mediated disease or condition, optionally wherein the DUX4 mediated disease or condition is facioscapulohumeral muscular dystrophy.

39. (canceled)

40. The engineered DUX4-targeting oligonucleotide of claim 8, wherein upon hybridization between the engineered DUX4-targeting oligonucleotide and the second RNA, the predicted thermal melting point is about 40 degrees Celsius to about 65 degrees Celsius.

41-42. (canceled)

43. A method of treating a disease or condition in a subject comprising administering to the subject a therapeutically effective amount the conjugate of claim 10.
